SEQUENCE LISTING (17 sequences)

SEQ ID NO: 1 - protein, 735 aa, Artificial Sequence [sequence omitted]

SEQ ID NO: 2 - protein, 735 aa, Artificial Sequence
  synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQVGNTGPATGDVNIQGVLPGMVWQNRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 735 aa, Artificial Sequence
  synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQSSITAPGTGDVNTQGCLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 735 aa, Artificial Sequence
  synthetic sequence
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQNGNTQPTTADVNHQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 5 - DNA, 2208 bp, Artificial Sequence
  synthetic sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccaggttggcaacacaggccctgctaccggtgatgtcaacatacaaggcgttcttccaggcatggtctggcagaacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 6 - DNA, 2208 bp, Artificial Sequence
  synthetic sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagtcttcgatcacggctccaggtaccggggatgtcaacacacaaggctgtcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 7 - DNA, 2208 bp, Artificial Sequence
  synthetic sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagaacggcaacacacaacccactaccgcagatgtcaaccaccaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - DNA, 2208 bp, Artificial Sequence
  synthetic sequence
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 9 - protein, 724 aa, Artificial Sequence
  synthetic sequence
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 10 - DNA, 2175 bp, Artificial Sequence
  synthetic sequence
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacgggctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 11 - protein, 738 aa, Artificial Sequence
  synthetic sequence
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 12 - DNA, 2217 bp, Artificial Sequence
  synthetic sequence
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 13 - protein, 736 aa, Artificial Sequence
  synthetic sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - DNA, 2211 bp, Artificial Sequence
  synthetic sequence
atggctgccgatggttatcttccagattggctcgaggacaaccttagtgaaggtattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 15 - protein, 738 aa, Artificial Sequence
  synthetic sequence
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 16 - protein, 738 aa, Artificial Sequence
  synthetic sequence
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 17 - DNA, 2217 bp, Artificial Sequence
  synthetic sequence
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagaggccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgtgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

(19) United States
(12) Patent Application Publication
(10) Pub. No.: US 2024/0287141 A1
(43) Pub. Date: Aug. 29, 2024

(54) CAPSID VARIANTS AND METHODS OF USING THE SAME

(71) Applicant: DYNO THERAPEUTICS, INC., Watertown, MA (US)

(72) Inventors: Sylvain Lapan, Brookline, MA (US); Hanna Levitin, Cambridge, MA (US)

(21) Appl. No.: 18/571,384

(22) PCT Filed: Jun. 17, 2022
(86) PCT No.: PCT/US2022/034057

§ 371 Date: Dec. 18, 2023

Related U.S. Application Data
(60) Provisional application No. 63/202,639, filed on Jun. 18, 2021.

(51) Int. Cl. C07K 14/005; A61K 48/00; C12N 15/86

(52) U.S. Cl. CPC C07K 14/005; A61K 48/0075; C12N 15/86; C12N 2750/14122; C12N 2750/14143

(57) ABSTRACT

The disclosure is directed in part to variant capsid polypeptides that can be used to deliver payloads.

### CROSS-REFERENCE TO RELATED APPLICATIONS

This application claims priority to U.S. Provisional Application No. 63/202,639, filed Jun. 18, 2021, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jun. 15, 2022, is named 257394_001102_ST25.txt and is 81,512 bytes in size.

### BACKGROUND

Dependoparvoviruses, e.g. adeno-associated dependoparvoviruses, e.g. adeno-associated viruses (AAVs), are of interest as vectors for delivering various payloads to cells, including in human subjects.

### SUMMARY

The present disclosure provides, in part, improved variant dependoparvovirus capsid proteins (e.g. variants of AAV2), such as VP1, VP2 and VP3 capsid proteins, methods of producing a dependoparvovirus, compositions for use in the same, as well as viral particles produced by the same. In some embodiments, the viral particles that include the variant capsid polypeptides have increased ocular biodistribution and/or transduction as compared to viral particles without the mutations in the capsid proteins. The present disclosure further provides variant capsid polypeptides, and virus particles comprising such variant capsid polypeptides that surprisingly transduce ocular tissue (e.g., retina tissue) after intravenous administration, and in particular, transduce ocular tissue to a much higher level degree after intravenous administration than an otherwise similar virus particle without the mutations described herein.

In some embodiments, the disclosure is directed, in part, to a nucleic acid comprising a sequence encoding a variant capsid protein as provided for herein. In some embodiments, the dependoparvovirus is an adeno-associated dependoparvovirus (AAV). In some embodiments, the AAV is AAV2, e.g., a variant of AAV2.

In some embodiments, the disclosure is directed, in part, to a capsid polypeptide described herein.

In some embodiments, the disclosure is directed, in part, to a dependoparvovirus particle comprising a nucleic acid described herein.

In some embodiments, the disclosure is directed, in part, to a vector, e.g., a plasmid, comprising a nucleic acid described herein.

In some embodiments, the disclosure is directed, in part, to a dependoparvovirus particle comprising a nucleic acid described herein (e.g., a nucleic acid comprising a sequence encoding a capsid polypeptide, such as VP1, wherein the encoding sequence comprises a change or mutation as provided herein.

In some embodiments, the disclosure is directed, in part, to dependoparvovirus particle comprising a variant capsid polypeptide comprising a polypeptide that has at least 70, 75, 80, 85, 90, 91, 92, 93, 94, 95, 96, 97, 98, 99%, or 100% identity to a VP1, VP2, or VP3 sequence of SEQ ID NO: 2, SEQ ID NO: 3, or SEQ ID NO: 4.

In some embodiments, the disclosure is directed, in part, to a nucleic acid molecule comprising SEQ ID NO: 5, SEQ ID NO: 6, or SEQ ID NO: 7, a fragment thereof, or a variant thereof having at least 70, 75, 80, 85, 90, 91, 92, 93, 94, 95, 96, 97, 98, 99, or 100% sequence identity thereto.

In some embodiments, the disclosure is directed, in part, to a vector comprising a nucleic acid described herein, e.g., a nucleic acid comprising a sequence encoding a capsid polypeptide, e.g. a VP1 polypeptide, wherein the encoding sequence comprises a change or mutation as provided for herein.

In some embodiments, the disclosure is directed, in part, to a cell, cell-free system, or other translation system comprising a nucleic acid or vector described herein, e.g., comprising a sequence encoding capsid polypeptide, such as VP1, wherein the capsid polypeptide encoding sequence comprises a change or mutation as provided for herein in the encoding sequence. In some embodiments, the cell, cell-free system, or other translation system comprises a dependoparvovirus particle described herein, e.g., wherein the particle comprises a nucleic acid comprising a sequence encoding a capsid polypeptide, such as a VP1 polypeptide, wherein the encoding sequence comprises a change or mutation as provided for herein.

In some embodiments, the disclosure is directed, in part, to a cell, cell-free system, or other translation system comprising a polypeptide described herein, wherein the polypeptide encoding sequence comprises a change or mutation as provided for herein. In some embodiments, the cell, cell-free system, or other translation system comprises a dependoparvovirus particle described herein, e.g., wherein the particle comprises a nucleic acid comprising a sequence encoding a VP1 polypeptide, wherein the VP1 encoding sequence comprises a change or mutation corresponding such as provided for herein.

In some embodiments, the disclosure is directed, in part, to a method of delivering a payload to a cell comprising contacting the cell with a dependoparvovirus particle comprising a nucleic acid described herein. In some embodiments, the disclosure is directed, in part, to a method of delivering a payload to a cell comprising contacting the cell with a dependoparvovirus particle comprising a capsid polypeptide described herein.

In some embodiments, the disclosure is directed, in part, to a method of making a dependoparvovirus particle, comprising providing a cell, cell-free system, or other translation system, comprising a nucleic acid described herein (e.g., a nucleic acid comprising a sequence encoding an AAV2 capsid variant as provided for herein); and cultivating the cell, cell-free system, or other translation system, under conditions suitable for the production of the dependoparvovirus particle, thereby making the dependoparvovirus particle. In some embodiments, the disclosure is directed, in part, to a method of making a dependoparvovirus particle described herein.

In some embodiments, the disclosure is directed, in part, to a method of making a dependoparvovirus particle, comprising providing a cell, cell-free system, or other translation system, comprising a polypeptide described herein; and cultivating the cell, cell-free system, or other translation system, under conditions suitable for the production of the dependoparvovirus particle, thereby making the dependoparvovirus particle. In some embodiments, the disclosure is directed, in part, to a method of making a dependoparvovirus particle described herein.

In some embodiments, the disclosure is directed, in part, to a dependoparvovirus particle made in a cell, cell-free system, or other translation system, wherein the cell, cell-free system, or other translation system comprises a nucleic acid encoding a dependoparvovirus comprising an capsid variant as provided for herein.

In some embodiments, the disclosure is directed, in part, to a method of treating a disease or condition in a subject, comprising administering to the subject a dependoparvovirus particle described herein in an amount effective to treat the disease or condition.

The invention is further described with reference to the following numbered embodiments.

### BRIEF DESCRIPTION OF THE DRAWINGS

FIG. 1. Diagram of tissues collected in each region of the eye. In the retina (left and center figures), peripheral and central retina samples from each of the superior, nasal, inferior and temporal regions of the retina were separately collected, macula was also separately collected. In each region, neural retina and choroid/RPE layers (center figure) were separately collected. In the TM/SC region (right figure), superior, temporal, nasal and inferior samples were separately collected.

FIG. 2A-C. Multisequence alignment of representative reference capsid VP1 polypeptides. Such alignment can be used to determine the amino acid positions which correspond to positions within different reference capsid polypeptides.

### ENUMERATED EMBODIMENTS

1. A variant capsid polypeptide comprising a polypeptide that has at least 70, 75, 80, 85, 90, 91, 92, 93, 94, 95, 96, 97, 98, 99% or 100% identity to a VP1, VP2, or VP3 sequence of SEQ ID NO: 2, SEQ ID NO: 3, or SEQ ID NO: 4.
2. The variant capsid polypeptide of embodiment 1, wherein the variant is the same serotype as the polypeptide of SEQ ID NO: 2, SEQ ID NO: 3, or SEQ ID NO: 4 (AAV2).
3. The variant capsid polypeptide of embodiment 1, wherein the variant is a different serotype as compared to the polypeptide of SEQ ID NO: 2, SEQ ID NO: 3, or SEQ ID NO: 4 (AAV2).
4. A variant capsid polypeptide any of the preceding embodiments, wherein the polypeptide comprises a variant of SEQ ID NO: 1, wherein the variant capsid polypeptide comprises a mutation that corresponds to a mutation at one or more positions of 585, 586, 587, 588, 589, 590, 591, 593, 597, 600, 608, as compared to SEQ ID NO: 1, optionally wherein the mutation comprises an insertion, a deletion, or a substitution.
5. The variant capsid polypeptide of any of the preceding embodiments, wherein the capsid polypeptide comprises a mutation that corresponds to a mutation at position 585 as compared to SEQ ID NO: 1.
6. The variant capsid polypeptide of any of the preceding embodiments, wherein the capsid polypeptide comprises a mutation that corresponds to a mutation at position 586 as compared to SEQ ID NO: 1.
7. The variant capsid polypeptide of any of the preceding embodiments, wherein the capsid polypeptide comprises a mutation that corresponds to a mutation at position 587 as compared to SEQ ID NO: 1.
8. The variant capsid polypeptide of any of the preceding embodiments, wherein the capsid polypeptide comprises a mutation that corresponds to a mutation at position 588 as compared to SEQ ID NO: 1.
9. The variant capsid polypeptide of any of the preceding embodiments, wherein the capsid polypeptide comprises a mutation that corresponds to a mutation at position 589 as compared to SEQ ID NO: 1.
10. The variant capsid polypeptide of any of the preceding embodiments, wherein the capsid polypeptide comprises a mutation that corresponds to a mutation at position 590 as compared to SEQ ID NO: 1.
11. The variant capsid polypeptide of any of the preceding embodiments, wherein the capsid polypeptide comprises a mutation that corresponds to a mutation at position 591 as compared to SEQ ID NO: 1.
12. The variant capsid polypeptide of any of the preceding embodiments, wherein the capsid polypeptide comprises a mutation that corresponds to a mutation at position 593 as compared to SEQ ID NO: 1.
13. The variant capsid polypeptide of any of the preceding embodiments, wherein the capsid polypeptide comprises a mutation that corresponds to a mutation at position 597 as compared to SEQ ID NO: 1.
14. The variant capsid polypeptide of any of the preceding embodiments, wherein the capsid polypeptide comprises a mutation that corresponds to a mutation at position 600 as compared to SEQ ID NO: 1.
15. The variant capsid polypeptide of any of the preceding embodiments, wherein the capsid polypeptide comprises a mutation that corresponds to a mutation at position 608 as compared to SEQ ID NO: 1.
16. The variant capsid polypeptide of any of the preceding embodiments, wherein the capsid polypeptide comprises one or more mutations (e.g., at least 2, at least 3, at least 4, at least 5, at least 6, or all mutations) that corresponds to a mutation at position 585, 588, 589, 590, 593, 597, and 608 as compared to SEQ ID NO: 1.
17. The variant capsid polypeptide of any of the preceding embodiments, wherein the capsid polypeptide comprises one or more mutations (e.g., at least 2, at least 3, at least 4, at least 5, at least 6, at least 7, at least 8, or all mutations) that corresponds to a mutation at position 585, 586,
587, 588, 589, 590, 591, 593, and 600 as compared to SEQ ID NO: 1. 18. The variant capsid polypeptide of any of the preceding embodiments, wherein the capsid polypeptide comprises one or more mutations (e.g., at least 2, at least 3, at least 4, or all mutations) that corresponds to a mutation at position 585, 588, 590, 591, and 597 as compared to SEQ ID NO: 1.
19. The variant capsid polypeptide of any of the preceding embodiments, wherein the capsid polypeptide comprises one or more mutations (e.g., at least 2, at least 3, at least 4, at least 5, at least 6, or all mutations) of R585V, R588T, Q589G, A590P, A593G, T597I, and D608N, as compared to SEQ ID NO: 1.
20. The variant capsid polypeptide of any of the preceding embodiments, wherein the capsid polypeptide comprises one or more mutations (e.g., at least 2, at least 3, at least 4, at least 5, at least 6, at least 7, at least 8, or all mutations) of R585S, G586S, N587I, R588T, Q589A, A590P, A591G, A593G, and V600C, as compared to SEQ ID NO: 1.
21. The variant capsid polypeptide of any of the preceding embodiments, wherein the capsid polypeptide comprises one or more mutations (e.g., at least 2, at least 3, at least 4, or all mutations) of R585N, R588T, A590P, A591T, and T597H, as compared to SEQ ID NO: 1.
22. A variant capsid polypeptide, comprising: (a) a polypeptide of any one of SEQ ID NO: 2, SEQ ID NO: 3, or SEQ ID NO: 4; (b) the VP2 or VP3 sequence of any one of SEQ ID NO: 2, SEQ ID NO: 3, or SEQ ID NO: 4; (c) a polypeptide comprising a sequence having at least 90%, at least 95%, at least 96%, at least 97%, at least 98%, or at least 99% identity thereto (e.g., to a polypeptide of (a) or (b)), wherein said polypeptide comprises at least one (e.g., one, two, three or more, e.g., all) of the mutations associated with any of SEQ ID NO: 2 through SEQ ID NO: 4, relative to SEQ ID NO: 1; or (d) a polypeptide having at least 1, but no more than 20, no more than 19, no more than 18, no more than 17, no more than 16, no more than 15, no more than 14, no more than 13, no more than 12, no more than 10, no more than 9, no more than 8, no more than 7, no more than 6, no more than 5, no more than 3, or no more than 2 amino acid mutations relative to the polypeptide of (a) or (b), wherein said polypeptide comprises at least one (e.g., one, two, three or more, e.g., all) of the mutations associated with any of SEQ ID NO: 2 through SEQ ID NO: 4, relative to SEQ ID NO: 1.
23. A variant capsid polypeptide comprising a VP1, VP2, or VP3, or any combination thereof, that is each at least, or about, 95, 96, 97, 98 or 99% identical to a polypeptide of SEQ ID NO: 2, SEQ ID NO: 3, or SEQ ID NO: 4 and comprises all the mutation differences of any of VAR-1 through VAR-3.
24. A variant capsid polypeptide comprising a VP1, VP2, or VP3, or any combination thereof, that each has about 1 to about 20 mutations as compared to a polypeptide of SEQ ID NO: 2, SEQ ID NO: 3, or SEQ ID NO: 4 and comprises all the mutation differences of any of VAR-1 through VAR-3.
25. A variant capsid polypeptide comprising a VP1, VP2, or VP3, or any combination thereof, that each has about 1 to about 10 mutations as compared to a polypeptide of SEQ ID NO: 2, SEQ ID NO: 3, or SEQ ID NO: 4 and comprises all the mutation differences of any of VAR-1 through VAR-3.
26. A variant capsid polypeptide comprising a VP1, VP2, or VP3, or any combination thereof, that each has about 1 to about 5 mutations as compared to a polypeptide of SEQ ID NO: 2, SEQ ID NO: 3, or SEQ ID NO: 4 and comprises all the mutation differences of any of VAR-1 through VAR-3.
27. A variant capsid polypeptide comprising a VP1, VP2 or VP3 sequence of SEQ ID NO: 2, SEQ ID NO: 3, or SEQ ID NO: 4.
28. A variant capsid polypeptide consisting of the VP1, VP2 or VP3 sequence of SEQ ID NO: 2, SEQ ID NO: 3, or SEQ ID NO: 4.
29. The variant capsid polypeptide of any of the preceding embodiments, wherein the variant capsid polypeptide is a VP1 polypeptide, a VP2 polypeptide or a VP3 polypeptide.
30. A nucleic acid molecule encoding the variant capsid polypeptide of any one of embodiments 1-29.
31. The nucleic acid molecule of embodiment 30, wherein the nucleic acid molecule comprises a sequence of SEQ ID NO: 5, 6, 7, a fragment thereof, or a variant thereof having at least 70, 75, 80, 85, 90, 91, 92, 93, 94, 95, 96, 97, 98, 99, or 100% sequence identity thereto.
32. The nucleic acid molecule of embodiment 31, wherein the fragment thereof encodes a VP2 capsid polypeptide or a VP3 capsid polypeptide.
33. A virus particle (e.g., adeno-associated virus (“AAV”) particle) comprising the variant AAV2 capsid polypeptide of any one of embodiments 1-29, or encoded by the nucleic acid molecule of any one of embodiments 30-32.
34. The virus particle of embodiment 33, comprising a nucleic acid comprising a transgene (e.g., payload) and one or more regulatory elements.
35. A virus particle of any one of embodiments 33-34, wherein said virus particle exhibits increased ocular transduction, e.g., as measured in a mouse or in NHP, e.g., as described herein, relative to wild-type AAV2 (E.g., a virus particle comprising capsid polypeptides of SEQ ID NO: 1 or encoded by SEQ ID NO: 8)
36. The virus particle of embodiment 35, wherein the increased ocular transduction is increased retinal transduction.
37. The virus particle of any one of embodiments 35-36, wherein the increased ocular transduction exhibited after systemic, e.g., intravenous, administration.
38. The nucleic acid molecule of any one of embodiments 30-32, wherein the nucleic acid molecule is double-stranded or single-stranded, and wherein the nucleic acid molecule is linear or circular, e.g., wherein the nucleic acid molecule is a plasmid.
39. A method of producing a virus particle comprising a variant capsid polypeptide, said method comprising introducing the nucleic acid molecule of any one of embodiments 30-32 or 38 into a cell (e.g., a HEK293 cell), and harvesting said virus particles therefrom.
40. A method of delivering a payload (e.g., a nucleic acid) to a cell comprising contacting the cell with (a) a dependoparvovirus particle comprising the variant capsid polypeptide of any one of embodiments 1-29 and a payload or (b) the virus particle of any one of embodiments 34-37.
41. The method of embodiment 40, wherein the cell is an ocular cell.
42. The method of embodiment 41, wherein the ocular cell is in the retina.
43. A method of delivering a payload (e.g., a nucleic acid) to a subject comprising administering to the subject a dependoparvovirus particle comprising a variant capsid polypeptide of any one of embodiments 1-29 and the payload, or administering to the subject the virus particle of any one of embodiments 34-37.
44. The method of embodiment 43, wherein the virus particle delivers the payload to the eye.
45. The method of embodiment 44, wherein the virus particle delivers the payload to the retina.
46. The method of any one of embodiments 40-45, wherein the virus particle is administered by systemic, e.g., intravenous, administration.
47. The variant capsid polypeptide of any one of embodiments 1-29, the virus particle of any one of embodiments 33-37, or the method of any one of embodiments 40-46, wherein the virus particle (e.g., the virus particle comprising the variant capsid polypeptide) delivers the payload to the eye with increased transduction in one or more regions of the eye as compared to a virus particle comprising capsid polypeptides of SEQ ID NO: 1.
48. The variant capsid polypeptide, virus particle or method of embodiment 47, wherein the one or more regions of the eye is the retina.
49. The variant capsid polypeptide, virus particle or method of embodiment 48, wherein the retina comprises non-macular retina.
50. The variant capsid polypeptide of any one of embodiments 1-29, the virus particle of any one of embodiments 33-37, or the method of any one of embodiments 40-46, wherein the virus particle (e.g., the virus particle comprising the variant capsid polypeptide) delivers the payload to the eye with increased transduction in one or more regions of the eye as compared to a virus particle comprising capsid polypeptides of SEQ ID NO: 1, and wherein the increase in transduction is at least 2-times, 4-times, 8-times, 16-times, 32-times, 64-times, 100-times, 150-times, 200-times, or 250-times greater as compared to a virus particle comprising capsid polypeptides of SEQ ID NO: 1.
51. The variant capsid polypeptide of any one of embodiments 1-29, the virus particle of any one of embodiments 33-37, or the method of any one of embodiments 40-46, wherein the virus particle (e.g., the virus particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times greater as compared to a virus particle comprising capsid polypeptides of SEQ ID NO: 1, and wherein the increase in transduction is specific to non-macular retina.
52. The variant capsid polypeptide, virus particle or method of any one of embodiments 39-51, wherein the administration to the subject is via systemic, e.g., intravenous injection.
53. A method of treating a disease or condition in a subject, comprising administering to the subject a dependoparvovirus particle in an amount effective to treat the disease or condition, wherein the dependoparvovirus particle is a virus particle comprising the capsid polypeptide of any one of embodiments 1-29, or encoded by the nucleic acid of any one of embodiments 30-31 or 38, or is the virus particle of any one of embodiments 33-37.
54. A method of treating a CNS and/or ocular disease or condition in a subject, comprising administering to the subject a dependoparvovirus particle in an amount effective to treat the disease or condition, wherein the dependoparvovirus particle is a virus particle comprising the capsid polypeptide of any one of embodiments 1-29, or encoded by the nucleic acid of any one of embodiments 30-31 or 38, or is the virus particle of any one of embodiments 33-37, optionally wherein the disease or condition is a neuronal ceroid lipofucsinosis (NCL).
55. A cell, cell-free system, or other translation system, comprising the capsid polypeptide, nucleic acid molecule, or virus particle of any one of embodiments 1-37 or 47-52.
56. A method of making a dependoparvovirus (e.g., an adeno-associated dependoparvovirus (AAV) particle, comprising:
   providing a cell, cell-free system, or other translation system, comprising a nucleic acid of any of embodiments 30-32 or 38; and
   cultivating the cell, cell-free system, or other translation system, under conditions suitable for the production of the dependoparvovirus particle,
   thereby making the dependoparvovirus particle.
57. The method of embodiment 56, wherein the cell, cell-free system, or other translation system comprises a second nucleic acid molecule and said second nucleic acid molecule is packaged in the dependoparvovirus particle.
58. The method of embodiment 57, wherein the second nucleic acid comprises a payload, e.g., a heterologous nucleic acid sequence encoding a therapeutic product.
59. The method of any one of embodiments 56-58, wherein the nucleic acid of any of embodiments 30-32 or 38 mediates the production of a dependoparvovirus particle which does not include said nucleic acid of any of embodiments 30-32 or 38.
60. The method of any one of embodiments 56-59, wherein the nucleic acid of any of embodiments 30-32 or 38 mediates the production of a dependoparvovirus particle at a level at least 10%, at least 20%, at least 50%, at least 100%, of the production level mediated by a nucleic acid encoding SEQ ID NO: 1, or at least 10% greater, at least 20% greater, at least 50% greater, or at least 100% greater than the production level mediated by a nucleic acid molecule encoding SEQ ID NO: 1.
61. A composition, e.g., a pharmaceutical composition, comprising the virus particle of any one of embodiments 33-37 or 47-52 or a virus particle produced by the method of any one of embodiments 39 or 56-60, and a pharmaceutically acceptable carrier.
62. The variant capsid polypeptide of any of embodiments 1-29, the nucleic acid molecule of any of embodiments 30-32 or 38, or the virus particle of any of embodiments 33-37 or 47-52 for use in treating a disease or condition in a subject.
63. The variant capsid polypeptide of any of embodiments 1-29, the nucleic acid molecule of any of embodiments 30-32 or 38, or the virus particle of any of embodiments 33-37 or 47-52 for use in the manufacture of a medicament for use in treating a disease or condition in a subject.

### DETAILED DESCRIPTION

The present disclosure is directed, in part, to the variant capsid variants that can be used to generate dependoparvovirus particles. In some embodiments, the particles have increased ocular transduction that can be used to deliver a transgene or molecule of interest to an eye with higher transduction efficiency in the eye as compared to a dependoparvovirus particle without the variant capsid polypeptides. Accordingly, provided herein are variant capsid polypeptides, nucleic acid molecules encoding the same, viral particles comprising the variant capsid polypeptides, and methods of using the same.

### Definitions

A, An, The: As used herein, the singular forms “a,” “an” and “the” include plural referents unless the context clearly dictates otherwise.

About, Approximately: As used herein, the terms “about” and “approximately” shall generally mean an acceptable degree of error for the quantity measured given the nature or precision of the measurements. Exemplary degrees of error are within 15 percent (%), typically, within 10%, and more typically, within 5% of a given value or range of values.

Dependoparvovirus capsid: As used herein, the term “dependoparvovirus capsid” refers to an assembled viral capsid comprising dependoparvovirus polypeptides. In some embodiments, a dependoparvovirus capsid is a functional dependoparvovirus capsid, e.g., is fully folded and/or assembled, is competent to infect a target cell, or remains stable (e.g., folded/assembled and/or competent to infect a target cell) for at least a threshold time.

Dependoparvovirus particle: As used herein, the term “dependoparvovirus particle” refers to an assembled viral capsid comprising dependoparvovirus polypeptides and a packaged nucleic acid, e.g., comprising a payload, one or more components of a dependoparvovirus genome (e.g., a whole dependoparvovirus genome), or both. In some embodiments, a dependoparvovirus particle is a functional dependoparvovirus particle, e.g., comprises a desired payload, is fully folded and/or assembled, is competent to infect a target cell, or remains stable (e.g., folded/assembled and/or competent to infect a target cell) for at least a threshold time.

Dependoparvovirus X particle/capsid: As used herein, the term “dependoparvovirus X particle/capsid” refers to a dependoparvovirus particle/capsid comprising at least one polypeptide or polypeptide encoding nucleic acid sequence derived from a naturally occurring dependoparvovirus X species. For example, a dependoparvovirus B particle refers to a dependoparvovirus particle comprising at least one polypeptide or polypeptide encoding nucleic acid sequence derived from a naturally occurring dependoparvovirus B sequence. Derived from, as used in this context, means having at least 70, 75, 80, 85, 90, 91, 92, 93, 94, 95, 96, 97, 98, 99, or 100% identity to the sequence in question. Correspondingly, an AAVX particle/capsid, as used herein, refers to an AAV particle/caspid comprising at least one polypeptide or polypeptide encoding nucleic acid sequence derived from a naturally occurring AAV X serotype. For example, an AAV2 particle refers to an AAV particle comprising at least one polypeptide or polypeptide encoding nucleic acid sequence derived from a naturally occurring AAV2 sequence.

Exogenous: As used herein, the term “exogenous” refers to a feature, sequence, or component present in a circumstance (e.g., in a nucleic acid, polypeptide, or cell) that does not naturally occur in said circumstance. For example, a nucleic acid sequence comprising a mutant capsid polypeptide or a nucleic acid molecule encoding the same may comprise an capsid polypeptide. Use of the term exogenous in this fashion means that the polypeptide or the nucleic acid molecule encoding a polypeptide comprising the mutation in question at this position does not occur naturally, e.g., is not present in AAV2, e.g., is not present in SEQ ID NO: 1.

Functional: As used herein in reference to a polypeptide component of a dependoparvovirus capsid (e.g., Cap (e.g., VP1, VP2, and/or VP3) or Rep), the term “functional” refers to a polypeptide which provides at least 50, 60, 70, 80, 90, or 100% of the activity of a naturally occurring version of that polypeptide component (e.g., when present in a host cell). For example, a functional VP1 polypeptide may stably fold and assemble into a dependoparvovirus capsid (e.g., that is competent for packaging and/or secretion). As used herein in reference to a dependoparvovirus capsid or particle, “functional” refers to a capsid or particle comprising one or more of the following production characteristics: comprises a desired payload, is fully folded and/or assembled, is competent to infect a target cell, or remains stable (e.g., folded/assembled and/or competent to infect a target cell) for at least a threshold time.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid monomer (e.g., a nucleotide and/or nucleoside); in some embodiments, “nucleic acid” refers to an oligonucleotide chain comprising individual nucleic acid monomers or a longer polynucleotide chain comprising many individual nucleic acid monomers. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid is, comprises, or consists of one or more modified, synthetic, or non-naturally occurring nucleotides. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid has a nucleotide sequence that encodes a functional gene product such as an RNA or protein. In some embodiments, a nucleic acid is partly or wholly single stranded; in some embodiments, a nucleic acid is partly or wholly double stranded.

Variant: As used herein, a “variant capsid polypeptide” refers to a polypeptide that differs from a reference sequence (e.g. SEQ ID NO: 1). The variant can, for example, comprise a mutation (e.g. substitution, deletion, or insertion). In some embodiments, the variant is about, or at least, 70%, 75%, 80%, 85%, 86%, 87%, 88%, 89%, 90%, 91%, 92%, 93%, 94%, 95%, 96%., 97%, 98%, or 99% identical to the reference sequence. In some embodiments, the reference sequence is a polypeptide comprising SEQ ID NO: 1.

### Capsid Polypeptides and Nucleic Acids Encoding the Same

The disclosure is directed, in part, to a nucleic acid comprising a sequence encoding a variant capsid polypeptide comprising a mutation (insertion, deletion, or substitution) as compared to a reference sequence. In some embodiments, the reference sequence is SEQ ID NO: 1. The disclosure is directed, in part, to variant capsid polypeptides comprising SEQ ID NO: 1 with one or more mutations as compared to SEQ ID NO: 1. The disclosure is further directed, in part, to variant capsid polypeptides comprising a reference sequence other than SEQ ID NO: 1 modified with one or more mutations corresponding to the mutations described herein. The mutation can be, for example, an insertion, deletion, or substitution as compared to the reference sequence. In some embodiments, the reference sequence is SEQ ID NO: 1.

In some embodiments, the capsid polypeptide comprises a mutation selected from Table 2. In some embodiments, the mutation selected from Table 2 is a substitution, e.g., a substitution of at least 2 or more residues, e.g., at least 6-10 residues, e.g., at least 7-10 residues, e.g., at least 8-10 residues, e.g., at least 9-10 residues, e.g., at least 10 residues that correspond to a substitution at positions between 585 and 608 as compared to SEQ ID NO: 1.

In some embodiments, the capsid polypeptide comprises a mutation that corresponds to a mutation at position 585, 586, 587, 588, 589, 590, 591, 593, 597, 600, 608, or any combination thereof according to SEQ ID NO: 1, optionally wherein the mutation comprises an insertion, a deletion or a substitution.

In some embodiments, the capsid polypeptide comprises a mutation that corresponds to a mutation at position 585 as compared to SEQ ID NO: 1. In some embodiments, the capsid polypeptide comprises a mutation that corresponds to a mutation at position 586 as compared to SEQ ID NO: 1. In some embodiments, the capsid polypeptide comprises a mutation that corresponds to a mutation at position 587 as compared to SEQ ID NO: 1. In some embodiments, the capsid polypeptide comprises a mutation that corresponds to a mutation at position 588 as compared to SEQ ID NO: 1. In some embodiments, the capsid polypeptide comprises a mutation that corresponds to a mutation at position 589 as compared to SEQ ID NO: 1. In some embodiments, the capsid polypeptide comprises a mutation that corresponds to a mutation at position 590 as compared to SEQ ID NO: 1. In some embodiments, the capsid polypeptide comprises a mutation that corresponds to a mutation at position 591 as compared to SEQ ID NO: 1. In some embodiments, the capsid polypeptide comprises a mutation that corresponds to a mutation at position 593 as compared to SEQ ID NO: 1. In some embodiments, the capsid polypeptide comprises a mutation that corresponds to a mutation at position 597 as compared to SEQ ID NO: 1. In some embodiments, the capsid polypeptide comprises a mutation that corresponds to a mutation at position 600 as compared to SEQ ID NO: 1. In some embodiments, the capsid polypeptide comprises a mutation that corresponds to a mutation at position 608 as compared to SEQ ID NO: 1. In some embodiments, the capsid polypeptide comprises a mutation that corresponds to a mutation at position 588 and 590 as compared to SEQ ID NO: 1.

In some embodiments, the mutation that corresponds to position 585 is a substitution. In some embodiments, the substitution is a naturally occurring amino acid. In some embodiments, the substitution is a valine, serine, or asparagine. In some embodiments, the substitution at position 585 is valine, e.g., R585V. In some embodiments, the substitution at position 585 is serine, e.g., R585S. In some embodiments, the substitution at position 585 is asparagine, e.g., R585N.

In some embodiments, the mutation that corresponds to position 586 is a substitution. In some embodiments, the substitution is a naturally occurring amino acid. In some embodiments, the substitution is a serine. In some embodiments, the substitution at position 586 is serine, e.g., G586S.

In some embodiments, the mutation that corresponds to position 587 is a substitution. In some embodiments, the substitution is a naturally occurring amino acid. In some embodiments, the substitution is an isoleucine. In some embodiments, the substitution at position 587 is isoleucine, e.g., N587I.

In some embodiments, the mutation that corresponds to position 588 is a substitution. In some embodiments, the substitution is a naturally occurring amino acid. In some embodiments, the substitution is a threonine. In some embodiments, the substitution at position 588 is threonine, e.g., R588T.

In some embodiments, the mutation that corresponds to position 589 is a substitution. In some embodiments, the substitution is a naturally occurring amino acid. In some embodiments, the substitution is an alanine or glycine. In some embodiments, the substitution at position 589 is alanine, e.g., Q589A. In some embodiments, the substitution at position 589 is glycine, e.g., Q589G.

In some embodiments, the mutation that corresponds to position 590 is a substitution. In some embodiments, the substitution is a naturally occurring amino acid. In some embodiments, the substitution is a proline. In some embodiments, the substitution at position 590 is proline, e.g., A590P.

In some embodiments, the mutation that corresponds to position 591 is a substitution. In some embodiments, the substitution is a naturally occurring amino acid. In some embodiments, the substitution is a threonine or glycine. In some embodiments, the substitution at position 591 is threonine, e.g., A591T. In some embodiments, the substitution at position 591 is glycine, e.g., A591G.

In some embodiments, the mutation that corresponds to position 593 is a substitution. In some embodiments, the substitution is a naturally occurring amino acid. In some embodiments, the substitution is a glycine. In some embodiments, the substitution at position 593 is glycine, e.g., A593G.

In some embodiments, the mutation that corresponds to position 597 is a substitution. In some embodiments, the substitution is a naturally occurring amino acid. In some embodiments, the substitution is an isoleucine or histidine. In some embodiments, the substitution at position 597 is isoleucine, e.g., T591I. In some embodiments, the substitution at position 597 is histidine, e.g., T591H.

In some embodiments, the mutation that corresponds to position 600 is a substitution. In some embodiments, the substitution is a naturally occurring amino acid. In some embodiments, the substitution is a cysteine. In some embodiments, the substitution at position 600 is cysteine, e.g., V600C.

In some embodiments, the mutation that corresponds to position 608 is a substitution. In some embodiments, the substitution is a naturally occurring amino acid. In some embodiments, the substitution is an asparagine. In some embodiments, the substitution at position 608 is asparagine, e.g., D608N.

In some embodiments, the capsid polypeptide comprises a mutation that corresponds to a mutation at position 585, 588, 589, 590, 593, 597, and 608 as compared to SEQ ID NO: 1.

In some embodiments, the capsid polypeptide comprises a mutation that corresponds to a mutation at position 585, 586, 587, 588, 589, 590, 591, 593, and 600 as compared to SEQ ID NO: 1.

In some embodiments, the capsid polypeptide comprises a mutation that corresponds to a mutation at position 585, 588, 590, 591, and 597 as compared to SEQ ID NO: 1.

In some embodiments, the capsid polypeptide comprises a mutation that corresponds to a R585V, R588T, Q589G, A590P, A593G, T597I, and D608N mutation as compared to SEQ ID NO: 1.

In some embodiments, the capsid polypeptide comprises a mutation that corresponds to a R585S, G586S, N587I, R588T, Q589A, A590P, A591G, A593G, and V600C mutation as compared to SEQ ID NO: 1.

In some embodiments, the capsid polypeptide comprises a mutation that corresponds to a R585N, R588T, A590P, A591T, and T597H mutation as compared to SEQ ID NO: 1.

In some embodiments, the capsid polypeptide comprises a mutation that corresponds to a R588T, and A590P mutation as compared to SEQ ID NO: 1.

In some embodiments, a nucleic acid molecule is provided. In some embodiments, the nucleic acid molecule has the sequence selected from Table 2. In some embodiments, the nucleic acid molecule has the sequence of SEQ ID NO: 5-7. In some embodiments, the nucleic acid molecule has the sequence of SEQ ID NO: 5. In some embodiments, the nucleic acid molecule has the sequence of SEQ ID NO: 6. In some embodiments, the nucleic acid molecule has the sequence of SEQ ID NO: 7.

In some embodiments, the nucleic acid molecule encodes a capsid polypeptide that comprises a mutation selected from Table 2. In some embodiments, the mutation selected from Table 2 is a substitution, e.g., a substitution of at least 2 or more residues, e.g., at least 6-10 residues, e.g., at least 7-10 residues, e.g., at least 8-10 residues, e.g., at least 9-10 residues, e.g., at least 10 residues that correspond to a substitution at positions between 585 and 608 as compared to SEQ ID NO: 1.

In some embodiments, the nucleic acid molecule encodes a capsid polypeptide that comprises a mutation that corresponds to a mutation at position 585, 586, 587, 588, 589, 590, 591, 593, 597, 600, 608, or any combination thereof according to SEQ ID NO: 1, optionally wherein the mutation comprises an insertion, a deletion or a substitution.

In some embodiments, the nucleic acid molecule encodes a capsid polypeptide that comprises a mutation that corresponds to a mutation at position 585 as compared to SEQ ID NO: 1. In some embodiments, the nucleic acid molecule encodes a capsid polypeptide that comprises a mutation that corresponds to a mutation at position 586 as compared to SEQ ID NO: 1. In some embodiments, the nucleic acid molecule encodes a capsid polypeptide that comprises a mutation that corresponds to a mutation at position 587 as compared to SEQ ID NO: 1. In some embodiments, the nucleic acid molecule encodes a capsid polypeptide that comprises a mutation that corresponds to a mutation at position 588 as compared to SEQ ID NO: 1. In some embodiments, the nucleic acid molecule encodes a capsid polypeptide that comprises a mutation that corresponds to a mutation at position 589 as compared to SEQ ID NO: 1. In some embodiments, the nucleic acid molecule encodes a capsid polypeptide that comprises a mutation that corresponds to a mutation at position 590 as compared to SEQ ID NO: 1. In some embodiments, the nucleic acid molecule encodes a capsid polypeptide that comprises a mutation that corresponds to a mutation at position 591 as compared to SEQ ID NO: 1. In some embodiments, the nucleic acid molecule encodes a capsid polypeptide that comprises a mutation that corresponds to a mutation at position 593 as compared to SEQ ID NO: 1. In some embodiments, the nucleic acid molecule encodes a capsid polypeptide that comprises a mutation that corresponds to a mutation at position 597 as compared to SEQ ID NO: 1. In some embodiments, the nucleic acid molecule encodes a capsid polypeptide that comprises a mutation that corresponds to a mutation at position 600 as compared to SEQ ID NO: 1. In some embodiments, the nucleic acid molecule encodes a capsid polypeptide that comprises a mutation that corresponds to a mutation at position 608 as compared to SEQ ID NO: 1.

In some embodiments, the nucleic acid molecule encodes a capsid polypeptide that comprises a mutation that corresponds to a mutation at position 588 and 590 as compared to SEQ ID NO: 1.

In some embodiments, the nucleic acid molecule encodes a capsid polypeptide that comprises a mutation that corresponds to a mutation at position 585, 588, 589, 590, 593, 597, and 608 as compared to SEQ ID NO: 1.

In some embodiments, the nucleic acid molecule encodes a capsid polypeptide that comprises a mutation that corresponds to a mutation at position 585, 586, 587, 588, 589, 590, 591, 593, and 600 as compared to SEQ ID NO: 1.

In some embodiments, the nucleic acid molecule encodes a capsid polypeptide that comprises a mutation that corresponds to a mutation at position 585, 588, 590, 591, and 597 as compared to SEQ ID NO: 1.

In some embodiments, the nucleic acid molecule encodes a capsid polypeptide that comprises a mutation that corresponds to a R585V, R588T, Q589G, A590P, A593G, T5971, and D608N mutation as compared to SEQ ID NO: 1.

In some embodiments, the nucleic acid molecule encodes a capsid polypeptide that comprises a mutation that corresponds to a R585S, G586S, N587I, R588T, Q589A, A590P, A591G, A593G, and V600C mutation as compared to SEQ ID NO: 1.

In some embodiments, the nucleic acid molecule encodes a capsid polypeptide that comprises a mutation that corresponds to a R585N, R588T, A590P, A591T, and T597H mutation as compared to SEQ ID NO: 1.

In some embodiments, the nucleic acid molecule encodes a capsid polypeptide that comprises a mutation that corresponds to a R588T, and A590P mutation as compared to SEQ ID NO: 1.

In some embodiments, the disclosure provides a capsid polypeptide (and nucleic acids encoding said capsid polypeptide) that comprises at least 1 of the mutation differences associated with any variant capsid polypeptide of Table 1 or comprises at least 1 mutation which corresponds to a mutation difference associated with any variant capsid polypeptide of Table 1. In aspects, the disclosure provides a capsid polypeptide (and nucleic acids encoding said capsid polypeptide) that comprises at least 2 mutation differences associated with any variant capsid polypeptide of Table 1 or comprises at least 2 mutations which corresponds to 2 mutation differences associated with any variant capsid polypeptide of Table 1. In aspects, the disclosure provides a capsid polypeptide (and nucleic acids encoding said capsid polypeptide) that comprises at least 3 mutation differences associated with any variant capsid polypeptide of Table 1 or comprises at least 3 mutations which corresponds to 3 mutation differences associated with any variant capsid polypeptide of Table 1. In aspects, the disclosure provides a capsid polypeptide (and nucleic acids encoding said capsid polypeptide) that comprises at least 4 mutation differences associated with any variant capsid polypeptide of Table 1 or comprises at least 4 mutations which corresponds to 4 mutation differences associated with any variant capsid polypeptide of Table 1. In aspects, the disclosure provides a capsid polypeptide (and nucleic acids encoding said capsid polypeptide) that comprises at least 5 mutation differences associated with any variant capsid polypeptide of Table 1 or comprises at least 5 mutations which corresponds to 5 mutation differences associated with any variant capsid polypeptide of Table 1. In aspects, the disclosure provides a capsid polypeptide (and nucleic acids encoding said capsid polypeptide) that comprises at least 6 mutation differences associated with any variant capsid polypeptide of Table 1 or comprises at least 6 mutations which corresponds to 6 mutation differences associated with any variant capsid polypeptide of Table 1. In aspects, the disclosure provides a capsid polypeptide (and nucleic acids encoding said capsid polypeptide) that comprises at least 7 mutation differences associated with any variant capsid polypeptide of Table 1 or comprises at least 7 mutations which corresponds to 7 mutation differences associated with any variant capsid polypeptide of Table 1. In aspects, the disclosure provides a capsid polypeptide (and nucleic acids encoding said capsid polypeptide) that comprises at least 8 mutation differences associated with any variant capsid polypeptide of Table 1 or comprises at least 8 mutations which corresponds to 8 mutation differences associated with any variant capsid polypeptide of Table 1. In aspects, the disclosure provides a capsid polypeptide (and nucleic acids encoding said capsid polypeptide) that comprises at least 9 mutation differences associated with any variant capsid polypeptide of Table 1 or comprises at least 9 mutations which corresponds to 9 mutation differences associated with any variant capsid polypeptide of Table 1. In aspects, the disclosure provides a capsid polypeptide (and nucleic acids encoding said capsid polypeptide) that comprises all of the mutation differences associated with any variant capsid polypeptide of Table 1 or comprises mutations which corresponds to all of the mutation differences associated with any variant capsid polypeptide of Table 1.

In any of the above aspects it will be understood that in variant capsid polypeptides described above where a number of mutation differences associated with or corresponding to the mutation differences of any variant capsid polypeptide of Table 1 is specified, the mutations may be chosen from any of the mutation differences associated with that variant capsid polypeptide. Thus, for example, with respect to the mutation differences of VAR-3 (with mutation differences R585N, R588T, A590P, A591T, T597H), where a variant capsid comprises 1 of the mutation differences, it may be R585N, R588T, A590P, A591T or T597H; likewise, where a variant capsid comprises 2 of the mutation differences, those two may be R585N and R588T, R585N and A590P, R585N and A591T, R585N and T597H, R588T and A590P, R588T and A591T, R588T and T597H, A590P and A591T, A590P and T597H, or A591T and T579H; likewise, where the variant comprises 3 of the mutation differences, those 3 may be R585N and R588T and A590P, R585N and R588T and A591T, R585N and R588T and T597H, R585N and A590P and A591P, R585N and A590P and T597H, R585N and A591T and T597H, R588T and A590P and A591T, R588T and A590P and T597H, R588T and A591T and T597H, or A590P and A591T and T597H; likewise, where the variant comprises 4 of the mutation differences, those 4 may be R585N and R588T and A590P and A591T, R585N and R588T and A590P and T597H, R588T and A590P and A591T and T597H, R585N and R588T and A591T and T597H, or R585N and A590P and A591T and T597H.

In some embodiments, disclosed herein is a capsid polypeptide comprising an R588T mutation and an A590P mutation (numbering according to SEQ ID NO: 1). In embodiments, the capsid polypeptide further comprises an A593G mutation (numbering according to SEQ ID NO: 1). In embodiments the capsid polypeptide comprising these mutations comprises a sequence comprising at least 1, least 2, at least 3, at least 4, at least 5, at least 6, at least 7, at least 8, at least 9, at least 10, at least 11, at least 12, at least, 13, at least 14, or at least 15 additional mutations relative to SEQ ID NO: 1 and comprising fewer than 35, fewer than 34, fewer than 33, fewer than 32, fewer than 31, fewer than 30, fewer than 29, fewer than 28, fewer than 27, fewer than 26, fewer than 25, fewer than 24, fewer than 23, fewer than 22, fewer than 21 or fewer than 20 additional mutations relative to SEQ ID NO: 1. In embodiments, the capsid polypeptide comprises a sequence comprising between zero and 14 additional mutations relative to SEQ ID NO: 1. In embodiments, the capsid polypeptide comprising these mutations comprises a sequence at least 90%, at least 95%, at least 96%, at least 97%, at least 98%, at least 99% identical to SEQ ID NO: 1. In embodiments said capsid polypeptide is a VP1 capsid polypeptide. In embodiments said capsid polypeptide is a VP2 capsid polypeptide. In embodiments said capsid polypeptide is a VP3 capsid polypeptide.

It will be understood by the skilled artisan that tables of the possible combinations of 2-9 mutation differences for each variant capsid polypeptide of Table 1 (up to the total number of mutation differences for that variant capsid polypeptide of Table 1) can be generated using routine skill and such tables for each of VAR-1 through VAR-3 is incorporated herein in its entirety. Such tables can be generated, for example, using the “combinations” method from the “itertools” package in Python, such method is hereby incorporated by reference in its entirety.

In embodiments, the variant capsid polypeptide comprises one or more mutation differences as described in Table 1 or which correspond to one or more mutation differences as described in Table 1. In embodiments, the variant capsid polypeptide is, but for the mutation differences described in or corresponding to the mutation differences as described in Table 1, at least 90%, at least 95%, 96%, 97%, 98%, 99%, or 100% identical to a reference AAV serotype described herein. In embodiments, the variant capsid polypeptide described herein is, but for the mutation differences of Table 1 or which correspond to the mutation differences of Table 1 comprised within such variant capsid polypeptide, at least 90%, at least 95%, 96%, 97%, 98%, 99%, or 100% identical to a capsid polypeptide of SEQ ID NO: 1 (e.g., a VP1, VP2 or VP3 sequence of SEQ ID NO: 1). In embodiments, the variant capsid polypeptide described herein is, but for the mutation differences of Table 1 or which correspond to the mutation differences of Table 1 comprised within such variant capsid polypeptide, at least 90%, at least 95%, 96%, 97%, 98%, 99%, or 100% identical to a capsid polypeptide of SEQ ID NO: 9 (e.g., a VP1, VP2 or VP3 sequence of SEQ ID NO: 9). In embodiments, the variant capsid polypeptide described herein is, but for the mutation differences of Table 1 or which correspond to the mutation differences of Table 1 comprised within such variant capsid polypeptide, at least 90%, at least 95%, 96%, 97%, 98%, 99%, or 100% identical to a capsid polypeptide of SEQ ID NO: 11 (e.g., a VP1, VP2 or VP3 sequence of SEQ ID NO: 11). In embodiments, the variant capsid polypeptide described herein is, but for the mutation differences of Table 1 or which correspond to the mutation differences of Table 1 comprised within such variant capsid polypeptide, at least 90%, at least 95%, 96%, 97%, 98%, 99%, or 100% identical to a capsid polypeptide of SEQ ID NO: 13 (e.g., a VP1, VP2 or VP3 sequence of SEQ ID NO: 13). In embodiments, the variant capsid polypeptide described herein is, but for the mutation differences of Table 1 or which correspond to the mutation differences of Table 1 comprised within such variant capsid polypeptide, at least 90%, at least 95%, 96%, 97%, 98%, 99%, or 100% identical to a capsid polypeptide of SEQ ID NO: 15 (e.g., a VP1, VP2 or VP3 sequence of SEQ ID NO: 15). In embodiments, the variant capsid polypeptide described herein is, but for the mutation differences of Table 1 or which correspond to the mutation differences of Table 1 comprised within such variant capsid polypeptide, at least 90%, at least 95%, 96%, 97%, 98%, 99%, or 100% identical to a capsid polypeptide of SEQ ID NO: 16 (e.g., a VP1, VP2 or VP3 sequence of SEQ ID NO: 16).

In some embodiments, the variant capsid polypeptide comprises: (a) a polypeptide of any one of SEQ ID NO: 2, SEQ ID NO: 3, or SEQ ID NO: 4; (b) the VP2 or VP3 sequence of any one of SEQ ID NO: 2, SEQ ID NO: 3, or SEQ ID NO: 4; (c) a polypeptide comprising a sequence having at least 90%, at least 95%, at least 96%, at least 97%, at least 98%, or at least 99% identity thereto (e.g., to a polypeptide of (a) or (b)), wherein said polypeptide comprises at least one (e.g., one, two, three or more, e.g., all) of the mutations associated with any of SEQ ID NO: 2 through SEQ ID NO: 4, relative to SEQ ID NO: 1; or (d) a polypeptide having at least 1, but no more than 20, no more than 19, no more than 18, no more than 17, no more than 16, no more than 15, no more than 14, no more than 13, no more than 12, no more than 10, no more than 9, no more than 8, no more than 7, no more than 6, no more than 5, no more than 3, or no more than 2 amino acid mutations relative to the polypeptide of (a) or (b), wherein said polypeptide comprises at least one (e.g., one, two, three or more, e.g., all) of the mutations associated with any of SEQ ID NO: 2 through SEQ ID NO: 4, relative to SEQ ID NO: 1.

In some embodiments, the variant capsid polypeptide comprises a VP1, VP2 VP3, or any combination thereof, that is each at least, or about, 95, 96, 97, 98 or 99% identical to a polypeptide of SEQ ID NO: 2, SEQ ID NO: 3, or SEQ ID NO: 4.

In some embodiments, the variant capsid polypeptide comprises a VP1, VP2, VP3, or any combination thereof, that each has about 1 to about 20 mutations as compared to a polypeptide of SEQ ID NO: 2, SEQ ID NO: 3, or SEQ ID NO: 4.

In some embodiments, the variant capsid polypeptide comprises a VP1, VP2, VP3, or any combination thereof, that each has about 1 to about 10 mutations as compared to a polypeptide of SEQ ID NO: 2, SEQ ID NO: 3, or SEQ ID NO: 4.

In some embodiments, the variant capsid polypeptide comprises a VP1, VP2, VP3, or any combination thereof, that each has about 1 to about 5 mutations as compared to a polypeptide of SEQ ID NO: 2, SEQ ID NO: 3, or SEQ ID NO: 4.

In some embodiments, the variant capsid polypeptide comprises a VP1, VP2 or VP3 sequence of SEQ ID NO: 2, SEQ ID NO: 3, or SEQ ID NO: 4.

In some embodiments, the variant capsid polypeptide consists of the VP1, VP2 or VP3 sequence of SEQ ID NO: 2, SEQ ID NO: 3, or SEQ ID NO: 4.

In some embodiments, the nucleic acid molecule encodes a capsid polypeptide as provided herein. In some embodiments, the nucleic acid molecule encodes a capsid polypeptide that is at least 70%, 75%, 80%, 85%, 90%, 91%, 92%, 93%, 94%, 95%, 96%, 97%, 98%, 99%, or 100% identical to a capsid polypeptide as provided herein.

In some embodiments, a capsid polypeptide is provided that comprises a capsid polypeptide that is at least 70%, 75%, 80%, 85%, 90%, 91%, 92%, 93%, 94%, 95%, 96%, 97%, 98%, 99%, or 100% identical to a capsid polypeptide as provided herein.

In some embodiments, the capsid polypeptide, or the reference polypeptide for purposes of % identity, comprises a sequence of SEQ ID NOs: 2, 3, or 4. In some embodiments, the capsid polypeptide, or the reference polypeptide for purposes of % identity, comprises a sequence of SEQ ID NO: 2. In some embodiments, the capsid polypeptide, or the reference polypeptide for purposes of % identity, comprises a sequence of SEQ ID NO: 3. In some embodiments, the capsid polypeptide, or the reference polypeptide for purposes of % identity, comprises a sequence of SEQ ID NO: 4.

In some embodiments, the nucleic acid molecule, or the reference nucleic acid molecule for purposes of % identity, comprises a nucleotide sequence of SEQ ID NOs: 5, 6, or 7. In some embodiments, the nucleic acid molecule, or the reference nucleic acid molecule for purposes of % identity, comprises a nucleotide sequence of SEQ ID NO: 5. In some embodiments, the nucleic acid molecule, or the reference nucleic acid molecule for purposes of % identity, comprises a nucleotide sequence of SEQ ID NO: 6. In some embodiments, the nucleic acid molecule, or the reference nucleic acid molecule for purposes of % identity, comprises a nucleotide sequence of SEQ ID NO: 7.

In some embodiments, the nucleic acid molecule, or the reference nucleic acid molecule for purposes of % identity, comprises a nucleotide sequence of SEQ ID NOs: 5, 6, or 7, that encodes a sequence of SEQ ID NOs: 2, 3, or 4. In some embodiments, the nucleic acid molecule, or the reference nucleic acid molecule for purposes of % identity, comprises a nucleotide sequence of SEQ ID NO: 5 that encodes a sequence of SEQ ID NO: 2. In some embodiments, the nucleic acid molecule, or the reference nucleic acid molecule for purposes of % identity, comprises a nucleotide sequence of SEQ ID NO: 6 that encodes a sequence of SEQ ID NO: 3. In some embodiments, the nucleic acid molecule, or the reference nucleic acid molecule for purposes of % identity, comprises a nucleotide sequence of SEQ ID NO: 7 that encodes a sequence of SEQ ID NO: 4.

In some embodiments, the capsid polypeptide, or the reference polypeptide for purposes of % identity, comprises a sequence of SEQ ID NOs: 2, 3, or 4 that is encoded by a nucleotide sequence of SEQ ID NOs: 5, 6, or 7. In some embodiments, the capsid polypeptide, or the reference polypeptide for purposes of % identity, comprises a sequence of SEQ ID NO: 2 that is encoded by a nucleotide sequence of SEQ ID NO: 5. In some embodiments, the capsid polypeptide, or the reference polypeptide for purposes of % identity, comprises a sequence of SEQ ID NO: 3 that is encoded by a nucleotide sequence of SEQ ID NO: 6. In some embodiments, the capsid polypeptide, or the reference polypeptide for purposes of % identity, comprises a sequence of SEQ ID NO: 4 that is encoded by a nucleotide sequence of SEQ ID NO: 7.

In some embodiments, the nucleic acid molecule comprises sequence that is at least 80%, at least 85%, at least 90%, at least 91%, at least 90%, at least 91%, at least 92%, at least 93%, at least 94%, at least 95%, at least 96%, at least 97%, at least 98%, or at least 99% identical to a nucleic acid molecule described herein, e.g., to any one of SEQ ID NO: 5 to SEQ ID NO: 7.

In some embodiments, the capsid polypeptide comprises a sequence that includes one, two, three, four, five, six (if present), seven (if present), eight (if present) or nine (if present) of the mutations associated with any one of VAR-1 through VAR-3 (e.g., as indicated in Table 1).

In some embodiments, the capsid polypeptide comprises a sequence that includes one, two, three, four, five, six (if present), seven (if present), eight (if present) or nine (if present) mutations that correspond to the mutations associated with any one of VAR-1 through VAR-3 (e.g., as indicated in Table 1). In some embodiments, the capsid polypeptide is otherwise at least 90%, 91%, 92%, 93%, 94%, 95%, 96%, 97%, 98%, 99%, identical to a reference capsid polypeptide sequence, e.g., as described herein, e.g., to SEQ ID NO:1. In some embodiments, the capsid polypeptide is otherwise 100% identical to a reference capsid polypeptide sequence, e.g., as described herein, e.g., to SEQ ID NO: 1.

In some embodiments, the capsid polypeptide comprises a sequence that includes all of the mutation differences associated with any one of VAR-1 through VAR-3 (e.g., as indicated in Table 1), and further includes no more than 30, no more than 20, no more than 10, no more than 9, no more than 8, no more than 7, no more than 6, no more than 5, no more than 4, no more than 3, no more than 2 or no more than 1 additional mutations relative to SEQ ID NO: 1.

In some embodiments, the capsid polypeptide is a VP1 capsid polypeptide. In embodiments, the capsid polypeptide is a VP2 capsid polypeptide. In embodiments, the capsid polypeptide is a VP3 capsid polypeptide. With respect to reference sequence SEQ ID NO: 1, a VP1 capsid polypeptide comprises amino acids 1-724 of SEQ ID NO: 1. With respect to reference sequence SEQ ID NO: 1, a VP2 capsid polypeptide comprises amino acids 138-724 of SEQ ID NO: 1. With respect to reference sequence SEQ ID NO: 1, a VP3 capsid polypeptide comprises amino acids 203-724 of SEQ ID NO: 1.

Table 1 lists information regarding exemplary variant dependoparvovirus particles comprising nucleic acids comprising the variant capsid regarding the ocular transduction properties and production characteristics of said non-limiting exemplary variants. Exemplary sequences of capsid polypeptides and nucleic acid molecules encoding the same are provided in Table 2.

Table 1. Transduction (after intravenous (“IV”) administration) and virus production of exemplary variant dependoparvovirus (e.g., AVV) particles comprising variant capsid polypeptides from the library experiment described in Example 1. Substitutions are notated as n ###N where “N” is the final amino acid, “n” is the reference amino acid and “###” is the reference amino acid position of SEQ ID NO:1; deletions are notated as n ###—where “-” indicates the deletion of “n” at position “###” of the reference sequence SEQ ID NO: 1; insertions are notated as ###_Naa_###_(n)y, where “###” are the amino acid positions in the reference sequence SEQ ID NO: 1 between which the insertion occurs, “Naa” refers to the length of the insertion (having “N” aminio acids) and “(n)y” providing the sequence of the insertion). Each individual Mutation Difference (e.g., within a row, each mutation in quotations (”) in column 7) and combinations of such individual mutation differences is sometimes referred to herein as a “mutation associated with VAR-X”, where VAR-X is the variant identifier listed in the “Name column.”

TABLE 1

| | | Retina | | |
|---|---|---|---|---|
| | | transduction | | |
| | SEQ ID | as compared | Virus | Mutation |
| | NO: of | to wild-type | Production | Differences |
| | VP1 | SEQ ID NO: 1 | as compared | as compared to |
| | capsid | after IV | to wild-type | SEQ ID NO: 1 |
| | poly- | administration | SEQ ID NO: 1 | (Collectively the |
| Name | peptide | (Log2) | (Log2) | “Mutation Set”) |
| VAR-1 | 2 | 8.25 | −0.04255732 | [‘R585V’, ‘R588T’, |
| | | | | ‘Q589G’, ‘A590P’, |
| | | | | ‘A593G’, ‘T597I’, |
| | | | | ‘D608N’] |
| VAR-2 | 3 | 7.96 | −0.880363717 | [‘R585S’, ‘G586S’, |
| | | | | ‘N587I’, ‘R588T’, |
| | | | | ‘Q589A’, ‘A590P’, |
| | | | | ‘A591G’, ‘A593G’, |
| | | | | ‘V600C’] |
| VAR-3 | 4 | 7.88 | −0.491465693 | [‘R585N’, ‘R588T’, |
| | | | | ‘A590P’, ‘A591T’, |
| | | | | ‘T597H’] |

Tables 4-6. Measured ocular region and liver biodistribution and transduction of virus particles comprising the capsid polypeptides of the indicated variant after intravitreal (IVT) administration (Table 4) or intravenous (IV) administration (Table 5; ocular regions/Table 6; liver) to non-human primates according to Example 2, relative to comparator virus particles comprising capsid polypeptides of wild-type AAV2 (e.g., capsid polypeptides of SEQ ID NO: 1) or in the case of liver properties, relative to comparator virus particles comprising capsid polypeptides of wild-type AAV2 (e.g., capsid polypeptides of SEQ ID NO: 1) or of wild-type AAV5 (e.g., capsid polypeptides of SEQ ID NO: 9). All values are log 2 relative to the indicated comparator delivered by the same administration route. “choroid” refers to the choroid layer from aggregated samples taken from all retina and macula tissue samples. “retina” refers to the neural retina layer from aggregated samples taken from all retina and macula tissue samples. “Non-macula retina” refers to the neural retina layer from aggregated samples taken from all retina, but not macula, tissue samples.

TABLE 4

| | | | | | | | | | Non-macula | | Non-macula | | Trabecular | |
|---|---|---|---|---|---|---|---|---|---|---|---|---|---|---|
| | Choroid | | Choroid | | Retina | | Retina | | Retina | | Retina | | Meshwork | |
| | biodistribution | | Transduction | | biodistribution | | Transduction | | biodistribution | | Transduction | | Transduction | |
| | by IVT | | by IVT | | by IVT | | by IVT | | by IVT | | by IVT | | by IVT | |
| | administration | | administration | | administration | | administration | | administration | | administration | | administration | |
| | (log2 relative | | (Log2 relative | | (log2 relative | | (Log2 relative | | (log2 relative | | (Log2 relative | | (Log2 relative | |
| | to AAV2) | | to wtAAV2) | | to AAV2) | | to wtAAV2) | | to AAV2) | | to wtAAV2) | | to wtAAV2) | |
| Variant | [std. dev.] | | [std. dev.] | | [std. dev.] | | [std. dev.] | | [std. dev.] | | [std. dev.] | | [std. dev.] | |
| VAR-1 | −0.88 | [0.26] | −0.64 | [0.24] | −0.3 | [0.06] | −1.37 | [0.35] | −0.3 | [0.06] | −1.38 | [0.35] | −3.58 | [0.33] |
| VAR-2 | −0.41 | [0.19] | −0.85 | [0.62] | 0.04 | [0.41] | −1.45 | [0.46] | 0.04 | [0.41] | −1.46 | [0.47] | −3.44 | [0.77] |
| VAR-3 | 0.11 | [0.24] | 0.25 | [0.24] | 0.58 | [0.2] | −0.68 | [0.37] | 0.58 | [0.2] | −0.68 | [0.37] | −2.43 | [0.52] |

TABLE 5

| | | | | | | | | | Non-macula | | Non-macula | | Trabecular | |
|---|---|---|---|---|---|---|---|---|---|---|---|---|---|---|
| | Choroid | | Choroid | | Retina | | Retina | | Retina | | Retina | | Meshwork | |
| | biodistribution | | Transduction | | biodistribution | | Transduction | | biodistribution | | Transduction | | Transduction | |
| | by IV | | by IV | | by IV | | by IV | | by IV | | by IV | | by IV | |
| | administration | | administration | | administration | | administration | | administration | | administration | | administration | |
| | (log2 relative | | (Log2 relative | | (log2 relative | | (Log2 relative | | (log2 relative | | (Log2 relative | | (Log2 relative | |
| | to AAV2) | | to wtAAV2) | | to AAV2) | | to wtAAV2) | | to AAV2) | | to wtAAV2) | | to wtAAV2) | |
| Variant | [std. dev.] | | [std. dev.] | | [std. dev.] | | [std. dev.] | | [std. dev.] | | [std. dev.] | | [std. dev.] | |
| VAR-1 | −4.8 | [2.17] | 4.65 | [0.27] | −0.09 | [3.31] | 5.4 | [0.56] | −0.09 | [3.31] | 5.34 | [0.59] | 0.68 | [2.79] |
| VAR-2 | −3.64 | [2.18] | 5.55 | [0.32] | 0.99 | [2.04] | 5.89 | [0.24] | 0.99 | [2.04] | 5.84 | [0.24] | 2.3 | [0.65] |
| VAR-3 | −0.27 | [1.45] | 4.91 | [0.2] | 0.31 | [3.3] | 5.42 | [0.31] | 0.31 | [3.3] | 5.36 | [0.3] | 2.66 | [0.77] |

TABLE 6

| | Liver | Liver | Liver | Liver |
|---|---|---|---|---|
| | biodistribution by | Transduction by | biodistribution by | Transduction by |
| | IV administration | IV administration | IV administration | IV administration |
| | (log2 relative | (Log2 relative | (log2 relative | (Log2 relative |
| | to AAV5) | to wtAAV5) | to AAV2) | to wtAAV2) |
| Variant | [std. dev.] | [std. dev.] | [std. dev.] | [std. dev.] |
| VAR-1 | −2.87 [0.26] | −2.43 [0.14] | −3.09 [0.26] | −4.44 [0.14] |
| VAR-2 | −2.79 [0.23] | −2.97 [0.23] | −3.01 [0.23] | −4.98 [0.23] |
| VAR-3 | −0.27 [0.38] | 0.11 [0.11] | −0.49 [0.38] | −1.92 [0.11] |

TABLE 7
Relative transduction rates in bulk brain tissue samples after
intravenous administration to non-human primates as described
in Example 2. All values are log2 relative to transduction rates
observed for virus particles comprising wild-type AAV2 capsid
polypeptides. Std. Dev. is the standard deviation in measurements
for the eight unique barcodes associated with each Variant.

| | | | Aggregated |
|---|---|---|---|
| | | | midbrain and |
| | Midbrain | Cerebellum | cerebellum |
| | transduction by | Transduction by | transduction by |
| | IV administration | IV administration | IV administration |
| | (log2 relative | (Log2 relative | (log2 relative |
| | to AAV2) | to wtAAV2) | to AAV2) |
| Variant | [std. dev.] | [std. dev.] | [std. dev.] |
| VAR-1 | 7.43 [0.33] | 4.36 [0.97] | 5.02 [0.43] |
| VAR-2 | 8.24 [0.85] | 4.94 [0.36] | 5.61 [0.52] |
| VAR-3 | 8.54 [0.25] | 5.52 [0.36] | 6.08 [0.18] |

TABLE 2

| | Amino Acid Sequence of VP1 | | |
|---|---|---|---|
| | capsid polypeptide (SEQ ID | | Mutation |
| | NO; starting amino acid of | | Differences |
| | VP2 is underlined; starting | | as compared |
| Capsid | amino acid of VP3 is in | Exemplary Nucleic Acid | to SEQ ID |
| Variant | bold. | Molecule Sequence (SEQ ID NO) | NO: 1 |
| VAR-1 | MAADGYLPDWLEDTLSEGIRQWWKLKPG | atggctgccgatggttatcttccagattggctcgagga | [‘R585V’, |
| | PPPPKPAERHKDDSRGLVLPGYKYLGPF | cactctctctgaaggaataagacagtggtggaagctc | ‘R588T’, |
| | NGLDKGEPVNEADAAALEHDKAYDRQLD | aaacctggcccaccaccaccaaagcccgcagagcgg | ‘Q589G’, |
| | SGDNPYLKYNHADAEFQERLKEDTSFGG | cataaggacgacagcaggggtcttgtgcttcctgggt | ‘A590P’, |
| | NLGRAVFQAKKRVLEPLGLVEEPVKTAP | acaagtacctcggacccttcaacggactcgacaaggg | ‘A593G’, |
| | GKKRPVEHSPVEPDSSSGTGKAGQQPAR | agagccggtcaacgaggcagacgccgcggccctcga | ‘T597I’, |
| | KRLNFGQTGDADSVPDPQPLGQPPAAPS | gcacgacaaagcctacgaccggcagctcgacagcgg | ‘D608N’] |
| | GLGTNTMATGSGAPMADNNEGADGVGNS | agacaacccgtacctcaagtacaaccacgccgacgcg | |
| | SGNWHCDSTWMGDRVITTSTRTWALPTY | gagtttcaggagcgccttaaagaagatacgtcttttgg | |
| | NNHLYKQISSQSGASNDNHYFGYSTPWG | gggcaacctcggacgagcagtcttccaggcgaaaaa | |
| | YFDFNRFHCHFSPRDWQRLINNNWGFRP | gagggttcttgaacctctgggcctggttgaggaacctg | |
| | KRLNFKLFNIQVKEVTQNDGTTTIANNL | ttaagacggctccgggaaaaaagaggccggtagagc | |
| | TSTVQVFTDSEYQLPYVLGSAHQGCLPP | actctcctgtggagccagactcctcctcgggaaccgga | |
| | FPADVFMVPQYGYLTLNNGSQAVGRSSF | aaggcgggccagcagcctgcaagaaaaagattgaat | |
| | YCLEYFPSQMLRTGNNFTFSYTFEDVPF | tttggtcagactggagacgcagactcagtacctgaccc | |
| | HSSYAHSQSLDRLMNPLIDQYLYYLSRT | ccagcctctcggacagccaccagcagccccctctggtc | |
| | NTPSGTTTQSRLQFSQAGASDIRDQSRN | tgggaactaatacgatggctacaggcagtggcgcacc | |
| | WLPGPCYRQQRVSKTSADNNNSEYSWTG | aatggcagacaataacgagggcgccgacggagtgg | |
| | ATKYHLNGRDSLVNPGPAMASHKDDEEK | gtaattcctcgggaaattggcattgcgattccacatgg | |
| | FFPQSGVLIFGKQGSEKTNVDIEKVMIT | atgggcgacagagtcatcaccaccagcacccgaacct | |
| | DEEEIRTTNPVATEQYGSVSTNLQVGNT | gggccctgcccacctacaacaaccacctctacaaacaa | |
| | GPATGDVNIQGVLPGMVWQNRDVYLQGP | atttccagccaatcaggagcctcgaacgacaatcacta | |
| | IWAKIPHTDGHFHPSPLMGGFGLKHPPP | ctttggctacagcaccccttgggggtattttgacttcaa | |
| | QILIKNTPVPANPSTTFSAAKFASFITQ | cagattccactgccacttttcaccacgtgactggcaaa | |
| | YSTGQVSVEIEWELQKENSKRWNPEIQY | gactcatcaacaacaactggggattccgacccaagag | |
| | TSNYNKSVNVDFTVDINGVYSEPRPIGT | actcaacttcaagctctttaacattcaagtcaaagaggt | |
| | RYLTRNL (SEQ ID NO: 2) | cacgcagaatgacggtacgacgacgattgccaataac | |
| | | cttaccagcacggttcaggtgtttactgactcggagta | |
| | | ccagctcccgtacgtcctcggctcggcgcatcaaggat | |
| | | gcctcccgccgttcccagcagacgtcttcatggtgcca | |
| | | cagtatggatacctcaccctgaacaacgggagtcagg | |
| | | cagtaggacgctcttcattttactgcctggagtactttcc | |
| | | ttctcagatgctgcgtaccggaaacaactttaccttcag | |
| | | ctacacttttgaggacgttcctttccacagcagctacgc | |
| | | tcacagccagagtctggaccgtctcatgaatcctctcat | |
| | | cgaccagtacctgtattacttgagcagaacaaacactc | |
| | | caagtggaaccaccacgcagtcaaggcttcagttttct | |
| | | caggccggagcgagtgacattcgggaccagtctagg | |
| | | aactggcttcctggaccctgttaccgccagcagcgagt | |
| | | atcaaagacatctgcggataacaacaacagtgaatac | |
| | | tcgtggactggagctaccaagtaccacctcaatggca | |
| | | gagactctctggtgaatccgggcccggccatggcaag | |
| | | ccacaaggacgatgaagaaaagttttttcctcagagc | |
| | | ggggttctcatctttgggaagcaaggctcagagaaaa | |
| | | caaatgtggacattgaaaaggtcatgattacagacga | |
| | | agaggaaatcaggacaaccaatcccgtggctacgga | |
| | | gcagtatggttctgtatctaccaacctccagGTTggc | |
| | | aacaCaGGCCcTgctaccgGTgatgtcaacaTac | |
| | | aaggcgttcttccaggcatggtctggcagAacagaga | |
| | | tgtgtaccttcaggggcccatctgggcaaagattccac | |
| | | acacggacggacattttcacccctctcccctcatgggt | |
| | | ggattcggacttaaacaccctcctccacagattctcatc | |
| | | aagaacaccccggtacctgcgaatccttcgaccacctt | |
| | | cagtgcggcaaagtttgcttccttcatcacacagtactc | |
| | | cacgggacaggtcagcgtggagatcgagtgggagct | |
| | | gcagaaggaaaacagcaaacgctggaatcccgaaat | |
| | | tcagtacacttccaactacaacaagtctgttaatgtgga | |
| | | ctttactgtggacactaatggcgtgtattcagagcctcg | |
| | | ccccattggcaccagatacctgactcgtaatctgtaa | |
| | | (SEQ ID NO: 5) | |
| VAR-2 | MAADGYLPDWLEDTLSEGIRQWWKLKPG | atggctgccgatggttatcttccagattggctcgagga | [‘R585S’, |
| | PPPPKPAERHKDDSRGLVLPGYKYLGPF | cactctctctgaaggaataagacagtggtggaagctc | ‘G586S’, |
| | NGLDKGEPVNEADAAALEHDKAYDRQLD | aaacctggcccaccaccaccaaagcccgcagagcgg | ‘N587I’, |
| | SGDNPYLKYNHADAEFQERLKEDTSFGG | cataaggacgacagcaggggtcttgtgcttcctgggt | ‘R588T’, |
| | NLGRAVFQAKKRVLEPLGLVEEPVKTAP | acaagtacctcggacccttcaacggactcgacaaggg | ‘Q589A’, |
| | GKKRPVEHSPVEPDSSSGTGKAGQQPAR | agagccggtcaacgaggcagacgccgcggccctcga | ‘A590P’, |
| | KRLNFGQTGDADSVPDPQPLGQPPAAPS | gcacgacaaagcctacgaccggcagctcgacagcgg | ‘A591G’, |
| | GLGTNTMATGSGAPMADNNEGADGVGNS | agacaacccgtacctcaagtacaaccacgccgacgcg | ‘A593G’, |
| | SGNWHCDSTWMGDRVITTSTRTWALPTY | gagtttcaggagcgccttaaagaagatacgtcttttgg | ‘V600C’] |
| | NNHLYKQISSQSGASNDNHYFGYSTPWG | gggcaacctcggacgagcagtcttccaggcgaaaaa | |
| | YFDFNRFHCHFSPRDWQRLINNNWGFRP | gagggttcttgaacctctgggcctggttgaggaacctg | |
| | KRLNFKLFNIQVKEVTQNDGTTTIANNL | ttaagacggctccgggaaaaaagaggccggtagagc | |
| | TSTVQVFTDSEYQLPYVLGSAHQGCLPP | actctcctgtggagccagactcctcctcgggaaccgga | |
| | FPADVFMVPQYGYLTLNNGSQAVGRSSF | aaggcgggccagcagcctgcaagaaaaagattgaat | |
| | YCLEYFPSQMLRTGNNFTFSYTFEDVPF | tttggtcagactggagacgcagactcagtacctgaccc | |
| | HSSYAHSQSLDRLMNPLIDQYLYYLSRT | ccagcctctcggacagccaccagcagccccctctggtc | |
| | NTPSGTTTQSRLQFSQAGASDIRDQSRN | tgggaactaatacgatggctacaggcagtggcgcacc | |
| | WLPGPCYRQQRVSKTSADNNNSEYSWTG | aatggcagacaataacgagggcgccgacggagtgg | |
| | ATKYHLNGRDSLVNPGPAMASHKDDEEK | gtaattcctcgggaaattggcattgcgattccacatgg | |
| | FFPQSGVLIFGKQGSEKTNVDIEKVMIT | atgggcgacagagtcatcaccaccagcacccgaacct | |
| | DEEEIRTTNPVATEQYGSVSTNLQSSIT | gggccctgcccacctacaacaaccacctctacaaacaa | |
| | APGTGDVNTQGCLPGMVWQDRDVYLQGP | atttccagccaatcaggagcctcgaacgacaatcacta | |
| | IWAKIPHTDGHFHPSPLMGGFGLKHPPP | ctttggctacagcaccccttgggggtattttgacttcaa | |
| | QILIKNTPVPANPSTTFSAAKFASFITQ | cagattccactgccacttttcaccacgtgactggcaaa | |
| | YSTGQVSVEIEWELQKENSKRWNPEIQY | gactcatcaacaacaactggggattccgacccaagag | |
| | TSNYNKSVNVDFTVDINGVYSEPRPIGT | actcaacttcaagctctttaacattcaagtcaaagaggt | |
| | RYLTRNL (SEQ ID NO: 3) | cacgcagaatgacggtacgacgacgattgccaataac | |
| | | cttaccagcacggttcaggtgtttactgactcggagta | |
| | | ccagctcccgtacgtcctcggctcggcgcatcaaggat | |
| | | gcctcccgccgttcccagcagacgtcttcatggtgcca | |
| | | cagtatggatacctcaccctgaacaacgggagtcagg | |
| | | cagtaggacgctcttcattttactgcctggagtactttcc | |
| | | ttctcagatgctgcgtaccggaaacaactttaccttcag | |
| | | ctacacttttgaggacgttcctttccacagcagctacgc | |
| | | tcacagccagagtctggaccgtctcatgaatcctctcat | |
| | | cgaccagtacctgtattacttgagcagaacaaacactc | |
| | | caagtggaaccaccacgcagtcaaggcttcagttttct | |
| | | caggccggagcgagtgacattcgggaccagtctagg | |
| | | aactggcttcctggaccctgttaccgccagcagcgagt | |
| | | atcaaagacatctgcggataacaacaacagtgaatac | |
| | | tcgtggactggagctaccaagtaccacctcaatggca | |
| | | gagactctctggtgaatccgggcccggccatggcaag | |
| | | ccacaaggacgatgaagaaaagttttttcctcagagc | |
| | | ggggttctcatctttgggaagcaaggctcagagaaaa | |
| | | caaatgtggacattgaaaaggtcatgattacagacga | |
| | | agaggaaatcaggacaaccaatcccgtggctacgga | |
| | | gcagtatggttctgtatctaccaacctccagTCTTCG | |
| | | aTcaCGGCTCcagGtaccgGGgatgtcaacacac | |
| | | aaggcTGtcttccaggcatggtctggcaggacagag | |
| | | atgtgtaccttcaggggcccatctgggcaaagattcca | |
| | | cacacggacggacattttcacccctctcccctcatgggt | |
| | | ggattcggacttaaacaccctcctccacagattctcatc | |
| | | aagaacaccccggtacctgcgaatccttcgaccacctt | |
| | | cagtgcggcaaagtttgcttccttcatcacacagtactc | |
| | | cacgggacaggtcagcgtggagatcgagtgggagct | |
| | | gcagaaggaaaacagcaaacgctggaatcccgaaat | |
| | | tcagtacacttccaactacaacaagtctgttaatgtgga | |
| | | ctttactgtggacactaatggcgtgtattcagagcctcg | |
| | | ccccattggcaccagatacctgactcgtaatctgtaa | |
| | | (SEQ ID NO: 6) | |
| VAR-3 | MAADGYLPDWLEDTLSEGIRQWWKLKPG | atggctgccgatggttatcttccagattggctcgagga | [‘R585N’, |
| | PPPPKPAERHKDDSRGLVLPGYKYLGPF | cactctctctgaaggaataagacagtggtggaagctc | ‘R588T’, |
| | NGLDKGEPVNEADAAALEHDKAYDRQLD | aaacctggcccaccaccaccaaagcccgcagagcgg | ‘A590P’, |
| | SGDNPYLKYNHADAEFQERLKEDTSFGG | cataaggacgacagcaggggtcttgtgcttcctgggt | ‘A591T’, |
| | NLGRAVFQAKKRVLEPLGLVEEPVKTAP | acaagtacctcggacccttcaacggactcgacaaggg | ‘T597H’] |
| | GKKRPVEHSPVEPDSSSGTGKAGQQPAR | agagccggtcaacgaggcagacgccgcggccctcga | |
| | KRLNFGQTGDADSVPDPQPLGQPPAAPS | gcacgacaaagcctacgaccggcagctcgacagcgg | |
| | GLGTNTMATGSGAPMADNNEGADGVGNS | agacaacccgtacctcaagtacaaccacgccgacgcg | |
| | SGNWHCDSTWMGDRVITTSTRTWALPTY | gagtttcaggagcgccttaaagaagatacgtcttttgg | |
| | NNHLYKQISSQSGASNDNHYFGYSTPWG | gggcaacctcggacgagcagtcttccaggcgaaaaa | |
| | YFDFNRFHCHFSPRDWQRLINNNWGFRP | gagggttcttgaacctctgggcctggttgaggaacctg | |
| | KRLNFKLFNIQVKEVTQNDGTTTIANNL | ttaagacggctccgggaaaaaagaggccggtagagc | |
| | TSTVQVFTDSEYQLPYVLGSAHQGCLPP | actctcctgtggagccagactcctcctcgggaaccgga | |
| | FPADVFMVPQYGYLTLNNGSQAVGRSSF | aaggcgggccagcagcctgcaagaaaaagattgaat | |
| | YCLEYFPSQMLRTGNNFTFSYTFEDVPF | tttggtcagactggagacgcagactcagtacctgaccc | |
| | HSSYAHSQSLDRLMNPLIDQYLYYLSRT | ccagcctctcggacagccaccagcagccccctctggtc | |
| | NTPSGTTTQSRLQFSQAGASDIRDQSRN | tgggaactaatacgatggctacaggcagtggcgcacc | |
| | WLPGPCYRQQRVSKTSADNNNSEYSWTG | aatggcagacaataacgagggcgccgacggagtgg | |
| | ATKYHLNGRDSLVNPGPAMASHKDDEEK | gtaattcctcgggaaattggcattgcgattccacatgg | |
| | FFPQSGVLIFGKQGSEKTNVDIEKVMIT | atgggcgacagagtcatcaccaccagcacccgaacct | |
| | DEEEIRTTNPVATEQYGSVSTNLQNGNT | gggccctgcccacctacaacaaccacctctacaaacaa | |
| | QPTTADVNHQGVLPGMVWQDRDVYLQGP | atttccagccaatcaggagcctcgaacgacaatcacta | |
| | IWAKIPHTDGHFHPSPLMGGFGLKHPPP | ctttggctacagcaccccttgggggtattttgacttcaa | |
| | QILIKNTPVPANPSTTFSAAKFASFITQ | cagattccactgccacttttcaccacgtgactggcaaa | |
| | YSTGQVSVEIEWELQKENSKRWNPEIQY | gactcatcaacaacaactggggattccgacccaagag | |
| | TSNYNKSVNVDFTVDINGVYSEPRPIGT | actcaacttcaagctctttaacattcaagtcaaagaggt | |
| | RYLTRNL (SEQ ID NO: 4) | cacgcagaatgacggtacgacgacgattgccaataac | |
| | | cttaccagcacggttcaggtgtttactgactcggagta | |
| | | ccagctcccgtacgtcctcggctcggcgcatcaaggat | |
| | | gcctcccgccgttcccagcagacgtcttcatggtgcca | |
| | | cagtatggatacctcaccctgaacaacgggagtcagg | |
| | | cagtaggacgctcttcattttactgcctggagtactttcc | |
| | | ttctcagatgctgcgtaccggaaacaactttaccttcag | |
| | | ctacacttttgaggacgttcctttccacagcagctacgc | |
| | | tcacagccagagtctggaccgtctcatgaatcctctcat | |
| | | cgaccagtacctgtattacttgagcagaacaaacactc | |
| | | caagtggaaccaccacgcagtcaaggcttcagttttct | |
| | | caggccggagcgagtgacattcgggaccagtctagg | |
| | | aactggcttcctggaccctgttaccgccagcagcgagt | |
| | | atcaaagacatctgcggataacaacaacagtgaatac | |
| | | tcgtggactggagctaccaagtaccacctcaatggca | |
| | | gagactctctggtgaatccgggcccggccatggcaag | |
| | | ccacaaggacgatgaagaaaagttttttcctcagagc | |
| | | ggggttctcatctttgggaagcaaggctcagagaaaa | |
| | | caaatgtggacattgaaaaggtcatgattacagacga | |
| | | agaggaaatcaggacaaccaatcccgtggctacgga | |
| | | gcagtatggttctgtatctaccaacctccagaACggc | |
| | | aacaCacaaCcCActaccgcagatgtcaacCACca | |
| | | aggcgttcttccaggcatggtctggcaggacagagat | |
| | | gtgtaccttcaggggcccatctgggcaaagattccaca | |
| | | cacggacggacattttcacccctctcccctcatgggtg | |
| | | gattcggacttaaacaccctcctccacagattctcatca | |
| | | agaacaccccggtacctgcgaatccttcgaccaccttc | |
| | | agtgcggcaaagtttgcttccttcatcacacagtactcc | |
| | | acgggacaggtcagcgtggagatcgagtgggagctg | |
| | | cagaaggaaaacagcaaacgctggaatcccgaaatt | |
| | | cagtacacttccaactacaacaagtctgttaatgtgga | |
| | | ctttactgtggacactaatggcgtgtattcagagcctcg | |
| | | ccccattggcaccagatacctgactcgtaatctgtaa | |
| | | (SEQ ID NO: 7) | |

In some embodiments, the capsid polypeptide has at least 85, 90, 91, 92, 93, 94, 95, 96, 97, 98, 99%, or 100% identity to a VP1, VP2, or VP3 sequence as provided in Table 2. In some embodiments, the capsid polypeptide has at least 85, 90, 91, 92, 93, 94, 95, 96, 97, 98, 99%, or 100% identity to a VP1, VP2, or VP3 sequence of SEQ ID NO: 2. In some embodiments, the capsid polypeptide has at least 85, 90, 91, 92, 93, 94, 95, 96, 97, 98, 99%, or 100% identity to a VP1, VP2, or VP3 sequence of SEQ ID NO: 3. In some embodiments, the capsid polypeptide has at least 85, 90, 91, 92, 93, 94, 95, 96, 97, 98, 99%, or 100% identity to a VP1, VP2, or VP3 sequence of SEQ ID NO: 4.

In some embodiments, the capsid polypeptide has a sequence of SEQ ID NO: 2-4. In some embodiments, the capsid polypeptide has a sequence of SEQ ID NO: 2. In some embodiments, the capsid polypeptide has a sequence of SEQ ID NO: 3. In some embodiments, the capsid polypeptide has a sequence of SEQ ID NO: 4.

In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence as provided in Table 2 and optionally includes at least one of, e.g., all of, the mutations associated with one of the variant capsid polypeptides of Table 2. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 2 and optionally includes at least one of, e.g., all of, the mutations associated with VAR-1 of Table 2.

In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 3 and optionally includes at least one of, e.g., all of, the mutations associated with VAR-2 of Table 2. In some embodiments, the nucleic acid molecule encodes a capsid polypeptide that has at least 85, 90, 91, 92, 93, 94, 95, 96, 97, 98, 99%, or 100% identity to a VP1, VP2, or VP3 sequence of SEQ ID NO: 4 and optionally includes at least one of, e.g., all of, the mutations associated with VAR-2 of Table 2.

In some embodiments, the capsid polypeptide comprises a reference capsid sequence, such as SEQ ID NO: 1, and at least, or about, 80%, 85%, 90%, or 95%, or 100% of the mutations (insertions, deletions, or substitutions) as shown in the Mutation Differences column of Table 1 of VAR-1, VAR-2, or VAR-3. In some embodiments, the reference capsid sequence comprises at least, about, or exactly, 80% of the mutations (insertions, deletions, or substitutions). In some embodiments, the reference capsid sequence comprises at least, about, or exactly, 85% of the mutations (insertions, deletions, or substitutions). In some embodiments, the reference capsid sequence comprises at least, about, or exactly, 90% of the mutations (insertions, deletions, or substitutions). In some embodiments, the reference capsid sequence comprises at least, about, or exactly, 95% of the mutations (insertions, deletions, or substitutions). In some embodiments, the reference capsid sequence comprises 100% of the mutations (insertions, deletions, or substitutions).

In some embodiments, the capsid polypeptide comprises a reference capsid sequence, such as SEQ ID NO: 1, and at least, or about, or exactly, 80%, 85%, 90%, or 95%, or 100% of one of the following groups of mutations (the terminology for these groups of mutations is provided for in the legend of Table 1 above): [′R585V′, ′R588T′, ′Q589G′, ′A590P′, ′A593G′, ′T597I′, ′D608N′]; [′R585S′, ′G586S′, ′N587I′, ′R588T′, ′Q589A′, ′A590P′, ′A591G′, ′A593G′, ′V600C′]; or [′R585N′, ′R588T′, ′A590P′, ′A591T′, ′T597H′].

In some embodiments, the capsid polypeptide comprises a reference capsid sequence, such as SEQ ID NO: 1, and at least, or about, or exactly, 80%, 85%, 90%, or 95%, or 100% of [′R585V′, ′R588T′, ′Q589G′, ′A590P′, ′A593G′, ′T597I′, ′D608N′]. In some embodiments, the capsid polypeptide comprises at least 6, or all of the mutations of [′R585V′, ′R588T′, ′Q589G′, ′A590P′, ′A593G′, ′T597I′, ′D608N′].

In some embodiments, the capsid polypeptide comprises a reference capsid sequence, such as SEQ ID NO: 1, and at least, or about, or exactly, 80%, 85%, 90%, or 95%, or 100% of [′R585S′, ′G586S′, ′N587I′, ′R588T′, ′Q589A′, ′A590P′, ′A591G′, ′A593G′, ′V600C′]. In some embodiments, the capsid polypeptide comprises at least 8, or all of the mutations of [′R585S′, ′G586S′, ′N587I′, ′R588T′, ′Q589A′, ′A590P′, ′A591G′, ′A593G′, ′V600C′].

In some embodiments, the capsid polypeptide comprises a reference capsid sequence, such as SEQ ID NO: 1, and at least, or about, or exactly, 80%, 85%, 90%, or 95%, or 100% of [′R585N′, ′R588T′, ′A590P′, ′A591T′, ′T597H′]. In some embodiments, the capsid polypeptide comprises at least 4, or all of the mutations of [′R585N′, ′R588T′, ′A590P′, ′A591T′, ′T597H′].

In embodiments, the nucleic acid molecule includes sequence encoding a variant capsid polypeptide described herein.

### Variant Capsids (Corresponding Positions)

The mutations to capsid polypeptide sequences described herein are described in relation to a position and/or amino acid at a position within a reference sequence, e.g., SEQ ID NO: 1. Thus, in some embodiments, the capsid polypeptides described herein are variant capsid polypeptides of the reference sequence, e.g., SEQ ID NO: 1, e.g., include capsid polypeptides comprising at least 80%, at least 85%, at least 90%, at least 91%, at least 92%, at least 93%, at least 94%, at least 95%, at least 96%, at least 97%, at least 98%, or at least 99 identity to the reference capsid polypeptide sequence (e.g., reference capsid polypeptide VP1, VP2 and/or VP3 sequence), e.g., SEQ ID NO: 1 (or VP2 or VP3 sequence comprised therein) and further include one or more, e.g., all of, the mutations described herein, e.g., the mutations associated with any one of VAR-1 through VAR-3 according to Table 1.

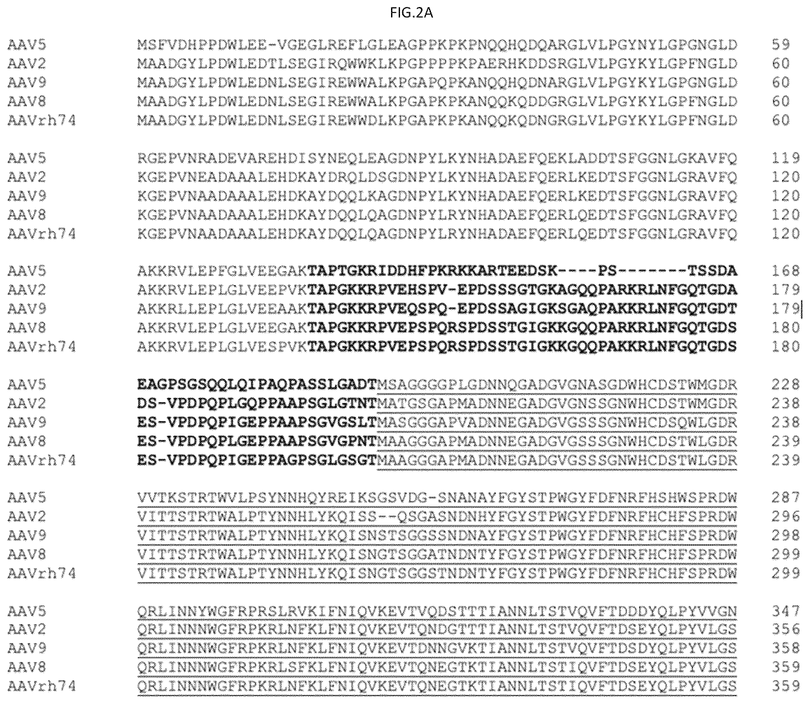

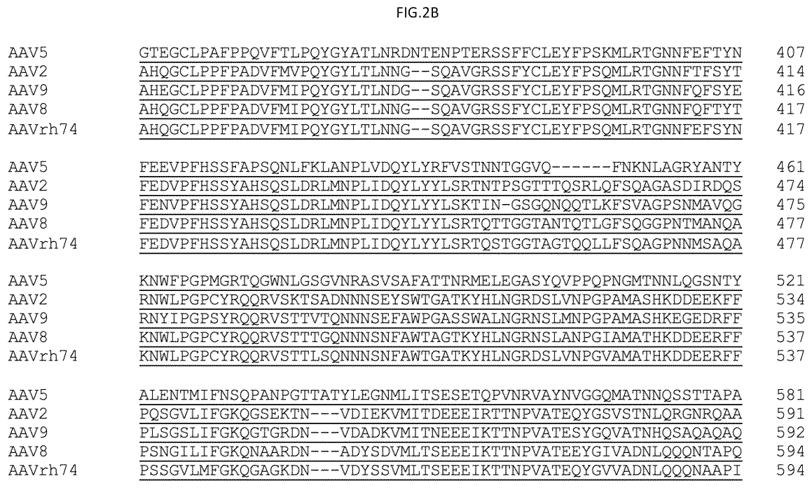

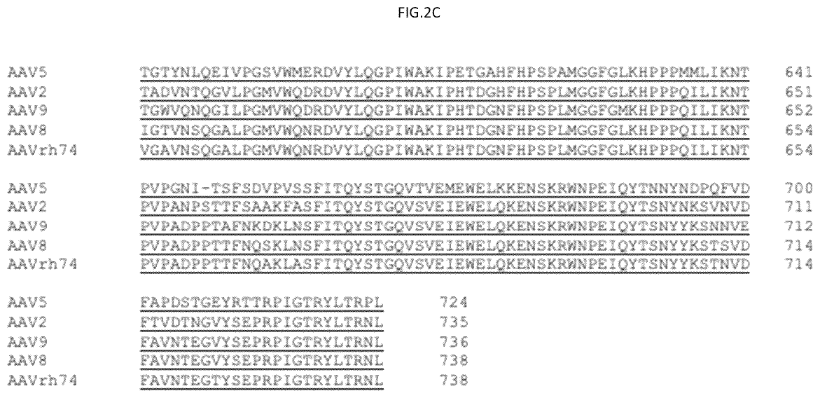

It will be understood by the skilled artisan, and without being bound by theory, that each amino acid position within a reference sequence corresponds to a position within the sequence of other capsid polypeptides such as capsid polypeptides derived from dependoparvoviruses with different serotypes. Such corresponding positions are identified using sequence alignment tools known in the art. A particularly preferred sequence alignment tool is Clustal Omega (Sievers F., et al., Mol. Syst. Biol. 7:359, 2011, DOI: 10.1038/msb.2011.75, incorporated herein by reference in its entirety). An alignment of exemplary reference capsid polypeptides is shown in FIG. 2A-2C. Thus, in some embodiments, the variant capsid polypeptides of the invention include variants of reference capsid polypeptides that include one or more mutations described herein in such reference capsid polypeptides at positions corresponding to the position of the mutation described herein in relation to a different reference capsid polypeptide. Thus, for example, a mutation described as XnnnY relative to SEQ ID NO: 1 (where X is the amino acid present at position nnn in SEQ ID NO: 1 and Y is the amino acid mutation at that position, e.g., described herein), the disclosure provides variant capsid polypeptides comprising at least 80%, at least 85%, at least 90%, at least 91%, at least 92%, at least 93%, at least 94%, at least 95%, at least 96%, at least 97%, at least 98%, or at least 99 identity to the reference capsid polypeptide sequence (e.g., reference capsid polypeptide VP1, VP2 and/or VP3 sequence) other than SEQ ID NO: 1 (or VP2 or VP3 sequence comprised therein) and further comprising the disclosed mutation at a position corresponding to position nnn of SEQ ID NO: 1 (e.g., comprising Y at the position in the new variant capsid polypeptide sequence that corresponds to position nnn of SEQ ID NO: 1). As described above, such corresponding position is determined using a sequence alignment tool, such as, for example, the clustal omega tool described above. Examples of corresponding amino acid positions of exemplary known AAV serotypes is provided in FIG. 2A-2C.

Thus, in embodiments, the disclosure provides capsid polypeptide sequences that are variants of a reference sequence other than SEQ ID NO: 1, e.g., a reference sequence other than SEQ ID NO: 1 as described herein, which include one or more mutation corresponding to the mutations described herein. In embodiments, such variants include mutations corresponding to all of the mutations associated with any one of VAR-1 through VAR-3 according to Table 1. As used herein, the term “corresponds to” as used in reference to a position in a sequence, such as an amino acid or nucleic acid sequence, can be used in reference to an entire capsid polypeptide or polynucleotide sequence, such as the full length sequence of the capsid polypeptide that comprises a VP1, VP2, and VP3 polypeptide, or a nucleic acid molecule encoding the same. In some embodiments, the term “corresponds to” can be used in reference to a region or domain of the capsid polypeptide. For example, a position that corresponds to a position in the VP1 section of the reference capsid polypeptide can correspond to the VP1 portion of the polypeptide of the variant capsid polypeptide. Thus, when aligning the two sequences to determine whether a position corresponds to another position the full length polypeptide can be used or domains (regions) can be used to determine whether a position corresponds to a specific position. In some embodiments, the region is the VP1 polypeptide. In some embodiments, the region is the VP2 polypeptide. In some embodiments, the region is the VP3 polypeptide. In some embodiments, when the reference polypeptide is the wild-type sequence (e.g., full length or region) of a certain serotype of AAV, the variant polypeptide can be of the same serotype with a mutation made at such corresponding position as compared to the reference sequence (e.g., full length or region). In some embodiments, the variant capsid polypeptide is a different serotype as compared to the reference sequence.

The variant capsid polypeptides described herein are optionally variants of reference capsids serotypes (e.g., comprising reference capsid polypeptides) known in the art. Non-limiting examples of such reference AAV serotypes (and associated reference capsid polypeptides) include AAV1, AAVrh10, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B- DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B- EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9, AAV9 K449R (or K449R AAV9), AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42- lb, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4- 8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVH-1/hu.1, AAVH-5/hu.3, AAVLG- 10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74 (also referred to as AAVrh74), AAVrh8R, AAVrh8R A586R mutant, AAVrh8R R533A mutant, AAAV, BAAV, caprine AAV, bovine AAV, AAVhE1.1, AAVhErL.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV- LK03, AAV-LK04, AAV-LK05, AAV-LK06, AAV-LK07, AAV-LK08, AAV-LK09, AAV- LK10, AAV-LK11, AAV-LK12, AAV-LK13, AAV-LK14, AAV-LK15, AAV-LK16, AAV- LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV- 8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr- E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd- H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clvl-10, AAV CLv1-2, AAV CLv-12, AAV CLv1-3, AAV CLv-13, AAV CLv1-4, AAV Clvl-7, AAV Clvl-8, AAV Clvl-9, AAV CLv- 2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv- M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, and/or AAVF9/HSC9, 7m8, Spark100, AAVMYO and variants thereof.

In some embodiments, the reference AAV capsid sequence comprises an AAV2 sequence. In some embodiments, the reference AAV capsid sequence comprises an AAV5 sequence. In some embodiments, the reference AAV capsid sequence comprises an AAV8 sequence. In some embodiments, the reference AAV capsid sequence comprises an AAV9 sequence. In some embodiments, the reference AAV capsid sequence comprises an AAVrh74 sequence. While not wishing to be bound by theory, it is understood that a reference AAV capsid sequence comprises a VP1 region. In certain embodiments, a reference AAV capsid sequence comprises a VP1, VP2 and/or VP3 region, or any combination thereof. A reference VP1 sequence may be considered synonymous with a reference AAV capsid sequence.

An exemplary reference sequence of SEQ ID NO: 1 (wild-type AAV2) is as follows:

(SEQ ID NO: 1)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVKTAPGKKRPVEHSP
VEPDSSSGTGKAGQQPARKRLNFGQTGDADSVPDPQPLGQPPAAPSGLGT
NTMATGSGAPMADNNEGADGVGNSSGNWHCDSTWMGDRVITTSTRTWALP
TYNNHLYKQISSQSGASNDNHYFGYSTPWGYFDFNRFHCHFSPRDWQRLI
NNNWGFRPKRLNFKLFNIQVKEVTQNDGTTTIANNLTSTVQVFTDSEYQL
PYVLGSAHQGCLPPFPADVFMVPQYGYLTLNNGSQAVGRSSFYCLEYFPS
QMLRTGNNFTFSYTFEDVPFHSSYAHSQSLDRLMNPLIDQYLYYLSRTNT
PSGTTTQSRLQFSQAGASDIRDQSRNWLPGPCYRQQRVSKTSADNNNSEY
SWTGATKYHLNGRDSLVNPGPAMASHKDDEEKFFPQSGVLIFGKQGSEKT
NVDIEKVMITDEEEIRTTNPVATEQYGSVSTNLQRGNRQAATADVNTQGV
LPGMVWQDRDVYLQGPIWAKIPHTDGHFHPSPLMGGFGLKHPPPQILIKN
TPVPANPSTTFSAAKFASFITQYSTGQVSVEIEWELQKENSKRWNPEIQY
TSNYNKSVNVDFTVDTNGVYSEPRPIGTRYLTRNL.

Unless otherwise noted, SEQ ID NO: 1 is the reference sequence. In the sequence above, the sequence found in VP1, VP2 and VP3 is underlined (e.g., a VP3 capsid polypeptide includes, e.g., consists of, amino acids corresponding to amino acids 203-735 of SEQ ID NO: 1), the sequence found in both VP1 and VP2 is in bold (e.g., a VP2 capsid polypeptide includes, e.g., consists of, the sequence corresponding to amino acids 138-735 of SEQ ID NO: 1) and the sequence that is not underlined or bold is found only in VP1 (e.g., a VP1 capsid polypeptide includes, e.g., consists of, amino acids corresponding to amino acids 1-735 of SEQ ID NO: 1).

An example nucleic acid sequence encoding SEQ ID NO: 1 is SEQ ID NO: 8:

(SEQ ID NO: 8)
ATGGCTGCCGATGGTTATCTTCCAGATTGGCTCGAGGACACTCTCTCTGA
AGGAATAAGACAGTGGTGGAAGCTCAAACCTGGCCCACCACCACCAAAGC
CCGCAGAGCGGCATAAGGACGACAGCAGGGGTCTTGTGCTTCCTGGGTAC
AAGTACCTCGGACCCTTCAACGGACTCGACAAGGGAGAGCCGGTCAACGA
GGCAGACGCCGCGGCCCTCGAGCACGACAAAGCCTACGACCGGCAGCTCG
ACAGCGGAGACAACCCGTACCTCAAGTACAACCACGCCGACGCGGAGTTT
CAGGAGCGCCTTAAAGAAGATACGTCTTTTGGGGGCAACCTCGGACGAGC
AGTCTTCCAGGCGAAAAAGAGGGTTCTTGAACCTCTGGGCCTGGTTGAGG
AACCTGTTAAGACGGCTCCGGGAAAAAAGAGGCCGGTAGAGCACTCTCCT
GTGGAGCCAGACTCCTCCTCGGGAACCGGAAAGGCGGGCCAGCAGCCTGC
AAGAAAAAGATTGAATTTTGGTCAGACTGGAGACGCAGACTCAGTACCTG
ACCCCCAGCCTCTCGGACAGCCACCAGCAGCCCCCTCTGGTCTGGGAACT
AATACGATGGCTACAGGCAGTGGCGCACCAATGGCAGACAATAACGAGGG
CGCCGACGGAGTGGGTAATTCCTCGGGAAATTGGCATTGCGATTCCACAT
GGATGGGCGACAGAGTCATCACCACCAGCACCCGAACCTGGGCCCTGCCC
ACCTACAACAACCACCTCTACAAACAAATTTCCAGCCAATCAGGAGCCTC
GAACGACAATCACTACTTTGGCTACAGCACCCCTTGGGGGTATTTTGACT
TCAACAGATTCCACTGCCACTTTTCACCACGTGACTGGCAAAGACTCATC
AACAACAACTGGGGATTCCGACCCAAGAGACTCAACTTCAAGCTCTTTAA
CATTCAAGTCAAAGAGGTCACGCAGAATGACGGTACGACGACGATTGCCA
ATAACCTTACCAGCACGGTTCAGGTGTTTACTGACTCGGAGTACCAGCTC
CCGTACGTCCTCGGCTCGGCGCATCAAGGATGCCTCCCGCCGTTCCCAGC
AGACGTCTTCATGGTGCCACAGTATGGATACCTCACCCTGAACAACGGGA
GTCAGGCAGTAGGACGCTCTTCATTTTACTGCCTGGAGTACTTTCCTTCT
CAGATGCTGCGTACCGGAAACAACTTTACCTTCAGCTACACTTTTGAGGA
CGTTCCTTTCCACAGCAGCTACGCTCACAGCCAGAGTCTGGACCGTCTCA
TGAATCCTCTCATCGACCAGTACCTGTATTACTTGAGCAGAACAAACACT
CCAAGTGGAACCACCACGCAGTCAAGGCTTCAGTTTTCTCAGGCCGGAGC
GAGTGACATTCGGGACCAGTCTAGGAACTGGCTTCCTGGACCCTGTTACC
GCCAGCAGCGAGTATCAAAGACATCTGCGGATAACAACAACAGTGAATAC
TCGTGGACTGGAGCTACCAAGTACCACCTCAATGGCAGAGACTCTCTGGT
GAATCCGGGCCCGGCCATGGCAAGCCACAAGGACGATGAAGAAAAGTTTT
TTCCTCAGAGCGGGGTTCTCATCTTTGGGAAGCAAGGCTCAGAGAAAACA
AATGTGGACATTGAAAAGGTCATGATTACAGACGAAGAGGAAATCAGGAC
AACCAATCCCGTGGCTACGGAGCAGTATGGTTCTGTATCTACCAACCTCC
AGAGAGGCAACAGACAAGCAGCTACCGCAGATGTCAACACACAAGGCGTT
CTTCCAGGCATGGTCTGGCAGGACAGAGATGTGTACCTTCAGGGGCCCAT
CTGGGCAAAGATTCCACACACGGACGGACATTTTCACCCCTCTCCCCTCA
TGGGTGGATTCGGACTTAAACACCCTCCTCCACAGATTCTCATCAAGAAC
ACCCCGGTACCTGCGAATCCTTCGACCACCTTCAGTGCGGCAAAGTTTGC
TTCCTTCATCACACAGTACTCCACGGGACAGGTCAGCGTGGAGATCGAGT
GGGAGCTGCAGAAGGAAAACAGCAAACGCTGGAATCCCGAAATTCAGTAC
ACTTCCAACTACAACAAGTCTGTTAATGTGGACTTTACTGTGGACACTAA
TGGCGTGTATTCAGAGCCTCGCCCCATTGGCACCAGATACCTGACTCGTA
ATCTGTAA.

An exemplary reference sequence of wild type AAV5, SEQ ID NO: 9 (wild-type AAV5), is as follows:

(SEQ ID NO: 9)
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAKTAPTGKRIDDHFPK
RKKARTEEDSKPSTSSDAEAGPSGSQQLQIPAQPASSLGADTMSAGGGGP
LGDNNQGADGVGNASGDWHCDSTWMGDRVVTKSTRTWVLPSYNNHQYREI
KSGSVDGSNANAYFGYSTPWGYFDFNRFHSHWSPRDWQRLINNYWGFRPR
SLRVKIFNIQVKEVTVQDSTTTIANNLTSTVQVFTDDDYQLPYVVGNGTE
GCLPAFPPQVFTLPQYGYATLNRDNTENPTERSSFFCLEYFPSKMLRTGN
NFEFTYNFEEVPFHSSFAPSQNLFKLANPLVDQYLYRFVSTNNTGGVQFN
KNLAGRYANTYKNWFPGPMGRTQGWNLGSGVNRASVSAFATTNRMELEGA
SYQVPPQPNGMTNNLQGSNTYALENTMIFNSQPANPGTTATYLEGNMLIT
SESETQPVNRVAYNVGGQMATNNQSSTTAPATGTYNLQEIVPGSVWMERD
VYLQGPIWAKIPETGAHFHPSPAMGGFGLKHPPPMMLIKNTPVPGNITSF
SDVPVSSFITQYSTGQVTVEMEWELKKENSKRWNPEIQYTNNYNDPQFVD
FAPDSTGEYRTTRPIGTRYLTRPL.

In the sequence above, the sequence found in VP1, VP2 and VP3 is underlined (e.g., a VP3 capsid polypeptide includes, e.g., consists of, amino acids corresponding to amino acids 193-725 of SEQ ID NO: 9), the sequence found in both VP1 and VP2 is in bold (e.g., a VP2 capsid polypeptide includes, e.g., consists of, the sequence corresponding to amino acids 137-725 of SEQ ID NO: 9) and the sequence that is not underlined or bold is found only in VP1 (e.g., a VP1 capsid polypeptide includes, e.g., consists of, amino acids corresponding to amino acids 1-725 of SEQ ID NO: 9).

An example nucleic acid sequence encoding SEQ ID NO: 9 is SEQ ID NO: 10:

(SEQ ID NO: 10)
ATGTCTTTTGTTGATCACCCTCCAGATTGGTTGGAAGAAGTTGGTGAAGG
TCTTCGCGAGTTTTTGGGCCTTGAAGCGGGCCCACCGAAACCAAAACCCA
ATCAGCAGCATCAAGATCAAGCCCGTGGTCTTGTGCTGCCTGGTTATAAC
TATCTCGGACCCGGAAACGGGCTCGATCGAGGAGAGCCTGTCAACAGGGC
AGACGAGGTCGCGCGAGAGCACGACATCTCGTACAACGAGCAGCTTGAGG
CGGGAGACAACCCCTACCTCAAGTACAACCACGCGGACGCCGAGTTTCAG
GAGAAGCTCGCCGACGACACATCCTTCGGGGGAAACCTCGGAAAGGCAGT
CTTTCAGGCCAAGAAAAGGGTTCTCGAACCTTTTGGCCTGGTTGAAGAGG
GTGCTAAGACGGCCCCTACCGGAAAGCGGATAGACGACCACTTTCCAAAA
AGAAAGAAGGCTCGGACCGAAGAGGACTCCAAGCCTTCCACCTCGTCAGA
CGCCGAAGCTGGACCCAGCGGATCCCAGCAGCTGCAAATCCCAGCCCAAC
CAGCCTCAAGTTTGGGAGCTGATACAATGTCTGCGGGAGGTGGCGGCCCA
TTGGGCGACAATAACCAAGGTGCCGATGGAGTGGGCAATGCCTCGGGAGA
TTGGCATTGCGATTCCACGTGGATGGGGGACAGAGTCGTCACCAAGTCCA
CCCGAACCTGGGTGCTGCCCAGCTACAACAACCACCAGTACCGAGAGATC
AAAAGCGGCTCCGTCGACGGAAGCAACGCCAACGCCTACTTTGGATACAG
CACCCCCTGGGGGTACTTTGACTTTAACCGCTTCCACAGCCACTGGAGCC
CCCGAGACTGGCAAAGACTCATCAACAACTACTGGGGCTTCAGACCCCGG
TCCCTCAGAGTCAAAATCTTCAACATTCAAGTCAAAGAGGTCACGGTGCA
GGACTCCACCACCACCATCGCCAACAACCTCACCTCCACCGTCCAAGTGT
TTACGGACGACGACTACCAGCTGCCCTACGTCGTCGGCAACGGGACCGAG
GGATGCCTGCCGGCCTTCCCTCCGCAGGTCTTTACGCTGCCGCAGTACGG
TTACGCGACGCTGAACCGCGACAACACAGAAAATCCCACCGAGAGGAGCA
GCTTCTTCTGCCTAGAGTACTTTCCCAGCAAGATGCTGAGAACGGGCAAC
AACTTTGAGTTTACCTACAACTTTGAGGAGGTGCCCTTCCACTCCAGCTT
CGCTCCCAGTCAGAACCTGTTCAAGCTGGCCAACCCGCTGGTGGACCAGT
ACTTGTACCGCTTCGTGAGCACAAATAACACTGGCGGAGTCCAGTTCAAC
AAGAACCTGGCCGGGAGATACGCCAACACCTACAAAAACTGGTTCCCGGG
GCCCATGGGCCGAACCCAGGGCTGGAACCTGGGCTCCGGGGTCAACCGCG
CCAGTGTCAGCGCCTTCGCCACGACCAATAGGATGGAGCTCGAGGGCGCG
AGTTACCAGGTGCCCCCGCAGCCGAACGGCATGACCAACAACCTCCAGGG
CAGCAACACCTATGCCCTGGAGAACACTATGATCTTCAACAGCCAGCCGG
CGAACCCGGGCACCACCGCCACGTACCTCGAGGGCAACATGCTCATCACC
AGCGAGAGCGAGACGCAGCCGGTGAACCGCGTGGCGTACAACGTCGGGGG
GCAGATGGCCACCAACAACCAGAGCTCCACCACTGCCCCCGCGACCGGCA
CGTACAACCTCCAGGAAATCGTGCCCGGCAGCGTGTGGATGGAGAGGGAC
GTGTACCTCCAAGGACCCATCTGGGCCAAGATCCCAGAGACGGGGGCGCA
CTTTCACCCCTCTCCGGCCATGGGGGGATTCGGACTCAAACACCCACCGC
CCATGATGCTCATCAAGAACACGCCTGTGCCCGGAAATATCACCAGCTTC
TCGGACGTGCCCGTCAGCAGCTTCATCACCCAGTACAGCACCGGGCAGGT
CACCGTGGAGATGGAGTGGGAGCTCAAGAAGGAAAACTCCAAGAGGTGGA
ACCCAGAGATCCAGTACACAAACAACTACAACGACCCCCAGTTTGTGGAC
TTTGCCCCGGACAGCACCGGGGAATACAGAACCACCAGACCTATCGGAAC
CCGATACCTTACCCGACCCCTTTAA.

An exemplary reference sequence of wild-type AAV8, SEQ ID NO: 11 (wild-type AAV8), is as follows:

(SEQ ID NO: 11)
MAADGYLPDWLEDNLSEGIREWWALKPGAPKPKANQQKQDDGRGLVLPGY
KYLGPENGLDKGEPVNAADAAALEHDKAYDQQLQAGDNPYLRYNHADAEF
QERLQEDTSFGGNLGRAVFQAKKRVLEPLGLVEEGAKTAPGKKRPVEPSP
QRSPDSSTGIGKKGQQPARKRLNFGQTGDSESVPDPQPLGEPPAAPSGVG
PNTMAAGGGAPMADNNEGADGVGSSSGNWHCDSTWLGDRVITTSTRTWAL
PTYNNHLYKQISNGTSGGATNDNTYFGYSTPWGYFDFNRFHCHFSPRDWQ
RLINNNWGFRPKRLSFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFQFTYTFEDVPFHSSYAHSQSLDRLMNPLIDQYLYYLSR
TQTTGGTANTQTLGFSQGGPNTMANQAKNWLPGPCYRQQRVSTTTGQNNN
SNFAWTAGTKYHLNGRNSLANPGIAMATHKDDEERFFPSNGILIFGKQNA
ARDNADYSDVMLTSEEEIKTTNPVATEEYGIVADNLQQQNTAPQIGTVNS
QGALPGMVWQNRDVYLQGPIWAKIPHTDGNFHPSPLMGGFGLKHPPPQIL
IKNTPVPADPPTTFNQSKLNSFITQYSTGQVSVEIEWELQKENSKRWNPE
IQYTSNYYKSTSVDFAVNTEGVYSEPRPIGTRYLTRNL.

In the sequence above, the sequence found in VP1, VP2 and VP3 is underlined (e.g., a VP3 capsid polypeptide includes, e.g., consists of, amino acids corresponding to amino acids 204-739 of SEQ ID NO: 11), the sequence found in both VP1 and VP2 is in bold (e.g., a VP2 capsid polypeptide includes, e.g., consists of, the sequence corresponding to amino acids 138-735 of SEQ ID NO: 11) and the sequence that is not underlined or bold is found only in VP1 (e.g., a VP1 capsid polypeptide includes, e.g., consists of, amino acids corresponding to amino acids 1-739 of SEQ ID NO: 11).

An example nucleic acid sequence encoding SEQ ID NO: 11 is SEQ ID NO: 12:

(SEQ ID NO: 12)
ATGGCTGCCGATGGTTATCTTCCAGATTGGCTCGAGGACAACCTCTCTGA
GGGCATTCGCGAGTGGTGGGCGCTGAAACCTGGAGCCCCGAAGCCCAAAG
CCAACCAGCAAAAGCAGGACGACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTGC
AGGGGGTGACAATCCGTACCTGCGGTATAACCACGCCGACGCCGAGTTTC
AGGAGCGTCTGCAAGAAGATACGTCTTTTGGGGGCAACCTCGGGCGAGCA
GTCTTCCAGGCCAAGAAGCGGGTTCTCGAACCTCTCGGTCTGGTTGAGGA
AGGCGCTAAGACGGCTCCTGGAAAGAAGAGACCGGTAGAGCCATCACCCC
AGCGTTCTCCAGACTCCTCTACGGGCATCGGCAAGAAAGGCCAACAGCCC
GCCAGAAAAAGACTCAATTTTGGTCAGACTGGCGACTCAGAGTCAGTTCC
AGACCCTCAACCTCTCGGAGAACCTCCAGCAGCGCCCTCTGGTGTGGGAC
CTAATACAATGGCTGCAGGCGGTGGCGCACCAATGGCAGACAATAACGAA
GGCGCCGACGGAGTGGGTAGTTCCTCGGGAAATTGGCATTGCGATTCCAC
ATGGCTGGGCGACAGAGTCATCACCACCAGCACCCGAACCTGGGCCCTGC
CCACCTACAACAACCACCTCTACAAGCAAATCTCCAACGGGACATCGGGA
GGAGCCACCAACGACAACACCTACTTCGGCTACAGCACCCCCTGGGGGTA
TTTTGACTTTAACAGATTCCACTGCCACTTTTCACCACGTGACTGGCAGC
GACTCATCAACAACAACTGGGGATTCCGGCCCAAGAGACTCAGCTTCAAG
CTCTTCAACATCCAGGTCAAGGAGGTCACGCAGAATGAAGGCACCAAGAC
CATCGCCAATAACCTCACCAGCACCATCCAGGTGTTTACGGACTCGGAGT
ACCAGCTGCCGTACGTTCTCGGCTCTGCCCACCAGGGCTGCCTGCCTCCG
TTCCCGGCGGACGTGTTCATGATTCCCCAGTACGGCTACCTAACACTCAA
CAACGGTAGTCAGGCCGTGGGACGCTCCTCCTTCTACTGCCTGGAATACT
TTCCTTCGCAGATGCTGAGAACCGGCAACAACTTCCAGTTTACTTACACC
TTCGAGGACGTGCCTTTCCACAGCAGCTACGCCCACAGCCAGAGCTTGGA
CCGGCTGATGAATCCTCTGATTGACCAGTACCTGTACTACTTGTCTCGGA
CTCAAACAACAGGAGGCACGGCAAATACGCAGACTCTGGGCTTCAGCCAA
GGTGGGCCTAATACAATGGCCAATCAGGCAAAGAACTGGCTGCCAGGACC
CTGTTACCGCCAACAACGCGTCTCAACGACAACCGGGCAAAACAACAATA
GCAACTTTGCCTGGACTGCTGGGACCAAATACCATCTGAATGGAAGAAAT
TCATTGGCTAATCCTGGCATCGCTATGGCAACACACAAAGACGACGAGGA
GCGTTTTTTTCCCAGTAACGGGATCCTGATTTTTGGCAAACAAAATGCTG
CCAGAGACAATGCGGATTACAGCGATGTCATGCTCACCAGCGAGGAAGAA
ATCAAAACCACTAACCCTGTGGCTACAGAGGAATACGGTATCGTGGCAGA
TAACTTGCAGCAGCAAAACACGGCTCCTCAAATTGGAACTGTCAACAGCC
AGGGGGCCTTACCCGGTATGGTCTGGCAGAACCGGGACGTGTACCTGCAG
GGTCCCATCTGGGCCAAGATTCCTCACACGGACGGCAACTTCCACCCGTC
TCCGCTGATGGGGGGCTTTGGCCTGAAACATCCTCCGCCTCAGATCCTGA
TCAAGAACACGCCTGTACCTGCGGATCCTCCGACCACCTTCAACCAGTCA
AAGCTGAACTCTTTCATCACGCAATACAGCACCGGACAGGTCAGCGTGGA
AATTGAATGGGAGCTGCAGAAGGAAAACAGCAAGCGCTGGAACCCCGAGA
TCCAGTACACCTCCAACTACTACAAATCTACAAGTGTGGACTTTGCTGTT
AATACAGAAGGCGTGTACTCTGAACCCCGCCCCATTGGCACCCGTTACCT
CACCCGTAATCTGTAA.

An exemplary reference sequence of wild-type AAV9, SEQ ID NO: 13 (wild-type AAV9), is as follows:

(SEQ ID NO: 13)
MAADGYLPDWLEDNLSEGIREWWALKPGAPQPKANQQHQDNARGLVLPGY
KYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGS
LTMASGGGAPVADNNEGADGVGSSSGNWHCDSQWLGDRVITTSTRTWALP
TYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDY
QLPYVLGSAHEGCLPPFPADVFMIPQYGYLTLNDGSQAVGRSSFYCLEYF
PSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSE
FAWPGASSWALNGRNSLMNPGPAMASHKEGEDRFFPLSGSLIFGKQGTGR
DNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIK
NTPVPADPPTAFNKDKLNSFITQYSTGQVSVEIEWELQKENSKRWNPEIQ
YTSNYYKSNNVEFAVNTEGVYSEPRPIGTRYLTRNL.

In the sequence above, the sequence found in VP1, VP2 and VP3 is underlined (e.g., a VP3 capsid polypeptide includes, e.g., consists of, amino acids corresponding to amino acids 203-737 of SEQ ID NO: 13), the sequence found in both VP1 and VP2 is in bold (e.g., a VP2 capsid polypeptide includes, e.g., consists of, the sequence corresponding to amino acids 138-737 of SEQ ID NO: 13) and the sequence that is not underlined or bold is found only in VP1 (e.g., a VP1 capsid polypeptide includes, e.g., consists of, amino acids corresponding to amino acids 1-737 of SEQ ID NO: 13).

An example nucleic acid sequence encoding SEQ ID NO: 13 is SEQ ID NO: 14:

(SEQ ID NO: 14)
ATGGCTGCCGATGGTTATCTTCCAGATTGGCTCGAGGACAACCTTAGTGA
AGGTATTCGCGAGTGGTGGGCTTTGAAACCTGGAGCCCCTCAACCCAAGG
CAAATCAACAACATCAAGACAACGCTCGAGGTCTTGTGCTTCCGGGTTAC
AAATACCTTGGACCCGGCAACGGACTCGACAAGGGGGAGCCGGTCAACGC
AGCAGACGCGGCGGCCCTCGAGCACGACAAGGCCTACGACCAGCAGCTCA
AGGCCGGAGACAACCCGTACCTCAAGTACAACCACGCCGACGCCGAGTTC
CAGGAGCGGCTCAAAGAAGATACGTCTTTTGGGGGCAACCTCGGGCGAGC
AGTCTTCCAGGCCAAAAAGAGGCTTCTTGAACCTCTTGGTCTGGTTGAGG
AAGCGGCTAAGACGGCTCCTGGAAAGAAGAGGCCTGTAGAGCAGTCTCCT
CAGGAACCGGACTCCTCCGCGGGTATTGGCAAATCGGGTGCACAGCCCGC
TAAAAAGAGACTCAATTTCGGTCAGACTGGCGACACAGAGTCAGTCCCAG
ACCCTCAACCAATCGGAGAACCTCCCGCAGCCCCCTCAGGTGTGGGATCT
CTTACAATGGCTTCAGGTGGTGGCGCACCAGTGGCAGACAATAACGAAGG
TGCCGATGGAGTGGGTAGTTCCTCGGGAAATTGGCATTGCGATTCCCAAT
GGCTGGGGGACAGAGTCATCACCACCAGCACCCGAACCTGGGCCCTGCCC
ACCTACAACAATCACCTCTACAAGCAAATCTCCAACAGCACATCTGGAGG
ATCTTCAAATGACAACGCCTACTTCGGCTACAGCACCCCCTGGGGGTATT
TTGACTTCAACAGATTCCACTGCCACTTCTCACCACGTGACTGGCAGCGA
CTCATCAACAACAACTGGGGATTCCGGCCTAAGCGACTCAACTTCAAGCT
CTTCAACATTCAGGTCAAAGAGGTTACGGACAACAATGGAGTCAAGACCA
TCGCCAATAACCTTACCAGCACGGTCCAGGTCTTCACGGACTCAGACTAT
CAGCTCCCGTACGTGCTCGGGTCGGCTCACGAGGGCTGCCTCCCGCCGTT
CCCAGCGGACGTTTTCATGATTCCTCAGTACGGGTATCTGACGCTTAATG
ATGGAAGCCAGGCCGTGGGTCGTTCGTCCTTTTACTGCCTGGAATATTTC
CCGTCGCAAATGCTAAGAACGGGTAACAACTTCCAGTTCAGCTACGAGTT
TGAGAACGTACCTTTCCATAGCAGCTACGCTCACAGCCAAAGCCTGGACC
GACTAATGAATCCACTCATCGACCAATACTTGTACTATCTCTCAAAGACT
ATTAACGGTTCTGGACAGAATCAACAAACGCTAAAATTCAGTGTGGCCGG
ACCCAGCAACATGGCTGTCCAGGGAAGAAACTACATACCTGGACCCAGCT
ACCGACAACAACGTGTCTCAACCACTGTGACTCAAAACAACAACAGCGAA
TTTGCTTGGCCTGGAGCTTCTTCTTGGGCTCTCAATGGACGTAATAGCTT
GATGAATCCTGGACCTGCTATGGCCAGCCACAAAGAAGGAGAGGACCGTT
TCTTTCCTTTGTCTGGATCTTTAATTTTTGGCAAACAAGGAACTGGAAGA
GACAACGTGGATGCGGACAAAGTCATGATAACCAACGAAGAAGAAATTAA
AACTACTAACCCGGTAGCAACGGAGTCCTATGGACAAGTGGCCACAAACC
ACCAGAGTGCCCAAGCACAGGCGCAGACCGGCTGGGTTCAAAACCAAGGA
ATACTTCCGGGTATGGTTTGGCAGGACAGAGATGTGTACCTGCAAGGACC
CATTTGGGCCAAAATTCCTCACACGGACGGCAACTTTCACCCTTCTCCGC
TGATGGGAGGGTTTGGAATGAAGCACCCGCCTCCTCAGATCCTCATCAAA
AACACACCTGTACCTGCGGATCCTCCAACGGCCTTCAACAAGGACAAGCT
GAACTCTTTCATCACCCAGTATTCTACTGGCCAAGTCAGCGTGGAGATCG
AGTGGGAGCTGCAGAAGGAAAACAGCAAGCGCTGGAACCCGGAGATCCAG
TACACTTCCAACTATTACAAGTCTAATAATGTTGAATTTGCTGTTAATAC
TGAAGGTGTATATAGTGAACCCCGCCCCATTGGCACCAGATACCTGACTC
GTAATCTGTAA.

An exemplary reference sequence of wild-type AAVrh74, SEQ ID NO: 15 (wild-type AAVrh74), is as follows:

(SEQ ID NO: 15)
MAADGYLPDWLEDNLSEGIREWWDLKPGAPKPKANQQKQDNGRGLVLPGY
KYLGPFNGLDKGEPVNAADAAALEHDKAYDQQLQAGDNPYLRYNHADAEF
QERLQEDTSFGGNLGRAVFQAKKRVLEPLGLVESPVKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSINDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYNFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFNQAKLASFITQYSTGQVSVEIEWELQKENSKRWNPE
IQYTSNYYKSTNVDFAVNTEGTYSEPRPIGTRYLTRNL.

An alternative exemplary reference sequence of SEQ ID NO: 16 (alternate wild-type AAVrh74) is as follows:

(SEQ ID NO: 16)
MAADGYLPDWLEDNLSEGIREWWDLKPGAPKPKANQQKQDNGRGLVLPGY
KYLGPFNGLDKGEPVNAADAAALEHDKAYDQQLQAGDNPYLRYNHADAEF
QERLQEDTSFGGNLGRAVFQAKKRVLEPLGLVESPVKTAPGKKRPVEPSP
QRSPDSSTGIGKKGQQPAKKRLNFGQTGDSESVPDPQPIGEPPAGPSGLG
SGTMAAGGGAPMADNNEGADGVGSSSGNWHCDSTWLGDRVITTSTRTWAL
PTYNNHLYKQISNGTSGGSINDNTYFGYSTPWGYFDFNRFHCHFSPRDWQ
RLINNNWGFRPKRLNFKLFNIQVKEVTQNEGTKTIANNLTSTIQVFTDSE
YQLPYVLGSAHQGCLPPFPADVFMIPQYGYLTLNNGSQAVGRSSFYCLEY
FPSQMLRTGNNFEFSYNFEDVPFHSSYAHSQSLDRLMNPLIDQYLYYLSR
TQSTGGTAGTQQLLFSQAGPNNMSAQAKNWLPGPCYRQQRVSTTLSQNNN
SNFAWTGATKYHLNGRDSLVNPGVAMATHKDDEERFFPSSGVLMFGKQGA
GKDNVDYSSVMLTSEEEIKTTNPVATEQYGVVADNLQQQNAAPIVGAVNS
QGALPGMVWQNRDVYLQGPIWAKIPHTDGNFHPSPLMGGFGLKHPPPQIL
IKNTPVPADPPTTFTKAKLASFITQYSTGQVSVEIEWELQKENSKRWNPE
IQYTSNYYKSTNVDFAVNTEGTYSEPRPIGTRYLTRNL.

In the sequences above (SEQ ID NO: 15 or SEQ ID NO: 16), the sequence found in VP1, VP2 and VP3 is underlined (e.g., a VP3 capsid polypeptide includes, e.g., consists of, amino acids corresponding to amino acids 204-739 of SEQ ID NO: 15), the sequence found in both VP1 and VP2 is in bold (e.g., a VP2 capsid polypeptide includes, e.g., consists of, the sequence corresponding to amino acids 137-739 of SEQ ID NO: 15) and the sequence that is not underlined or bold is found only in VP1 (e.g., a VP1 capsid polypeptide includes, e.g., consists of, amino acids corresponding to amino acids 1-739 of SEQ ID NO: 15).

An example nucleic acid sequence encoding SEQ ID NO: 15 is SEQ ID NO: 17.

(SEQ ID NO: 17)
ATGGCTGCCGATGGTTATCTTCCAGATTGGCTCGAGGACAACCTCTCTGA
GGGCATTCGCGAGTGGTGGGACCTGAAACCTGGAGCCCCGAAACCCAAAG
CCAACCAGCAAAAGCAGGACAACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCC
AAGCGGGTGACAATCCGTACCTGCGGTATAATCACGCCGACGCCGAGTTT
CAGGAGCGTCTGCAAGAAGATACGTCTTTTGGGGGCAACCTCGGGCGCGC
AGTCTTCCAGGCCAAAAAGCGGGTTCTCGAACCTCTGGGCCTGGTTGAAT
CGCCGGTTAAGACGGCTCCTGGAAAGAAGAGGCCGGTAGAGCCATCACCC
CAGCGCTCTCCAGACTCCTCTACGGGCATCGGCAAGAAAGGCCAGCAGCC
CGCAAAAAAGAGACTCAATTTTGGGCAGACTGGCGACTCAGAGTCAGTCC
CCGACCCTCAACCAATCGGAGAACCACCAGCAGGCCCCTCTGGTCTGGGA
TCTGGTACAATGGCTGCAGGCGGTGGCGCTCCAATGGCAGACAATAACGA
AGGCGCCGACGGAGTGGGTAGTTCCTCAGGAAATTGGCATTGCGATTCCA
CATGGCTGGGCGACAGAGTCATCACCACCAGCACCCGCACCTGGGCCCTG
CCCACCTACAACAACCACCTCTACAAGCAAATCTCCAACGGGACCTCGGG
AGGAAGCACCAACGACAACACCTACTTCGGCTACAGCACCCCCTGGGGGT
ATTTTGACTTCAACAGATTCCACTGCCACTTTTCACCACGTGACTGGCAG
CGACTCATCAACAACAACTGGGGATTCCGGCCCAAGAGGCTCAACTTCAA
GCTCTTCAACATCCAAGTCAAGGAGGTCACGCAGAATGAAGGCACCAAGA
CCATCGCCAATAACCTTACCAGCACGATTCAGGTCTTTACGGACTCGGAA
TACCAGCTCCCGTACGTGCTCGGCTCGGCGCACCAGGGCTGCCTGCCTCC
GTTCCCGGCGGACGTCTTCATGATTCCTCAGTACGGGTACCTGACTCTGA
ACAATGGCAGTCAGGCTGTGGGCCGGTCGTCCTTCTACTGCCTGGAGTAC
TTTCCTTCTCAAATGCTGAGAACGGGCAACAACTTTGAATTCAGCTACAA
CTTCGAGGACGTGCCCTTCCACAGCAGCTACGCGCACAGCCAGAGCCTGG
ACCGGCTGATGAACCCTCTCATCGACCAGTACTTGTACTACCTGTCCCGG
ACTCAAAGCACGGGCGGTACTGCAGGAACTCAGCAGTTGCTATTTTCTCA
GGCCGGGCCTAACAACATGTCGGCTCAGGCCAAGAACTGGCTACCCGGTC
CCTGCTACCGGCAGCAACGTGTCTCCACGACACTGTCGCAGAACAACAAC
AGCAACTTTGCCTGGACGGGTGCCACCAAGTATCATCTGAATGGCAGAGA
CTCTCTGGTGAATCCTGGCGTTGCCATGGCTACCCACAAGGACGACGAAG
AGCGATTTTTTCCATCCAGCGGAGTCTTAATGTTTGGGAAACAGGGAGCT
GGAAAAGACAACGTGGACTATAGCAGCGTGATGCTAACCAGCGAGGAAGA
AATAAAGACCACCAACCCAGTGGCCACAGAACAGTACGGCGTGGTGGCCG
ATAACCTGCAACAGCAAAACGCCGCTCCTATTGTAGGGGCCGTCAATAGT
CAAGGAGCCTTACCTGGCATGGTGTGGCAGAACCGGGACGTGTACCTGCA
GGGTCCCATCTGGGCCAAGATTCCTCATACGGACGGCAACTTTCATCCCT
CGCCGCTGATGGGAGGCTTTGGACTGAAGCATCCGCCTCCTCAGATCCTG
ATTAAAAACACACCTGTTCCCGCGGATCCTCCGACCACCTTCAATCAGGC
CAAGCTGGCTTCTTTCATCACGCAGTACAGTACCGGCCAGGTCAGCGTGG
AGATCGAGTGGGAGCTGCAGAAGGAGAACAGCAAACGCTGGAACCCAGAG
ATTCAGTACACTTCCAACTACTACAAATCTACAAATGTGGACTTTGCTGT
CAATACTGAGGGTACTTATTCCGAGCCTCGCCCCATTGGCACCCGTTACC
TCACCCGTAATCTGTAA.

The present disclosure refers to structural capsid proteins (including VP1, VP2 and VP3) which are encoded by capsid (Cap) genes. These capsid proteins form an outer protein structural shell (i.e. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins. Where the Met/AA-clipping is incomplete, a mixture of one or more (one, two or three) VP capsid proteins comprising the viral capsid can be produced, some of which include a Met1/AA1 amino acid (Met+/AA+) and some of which lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods.2017 Oct.28(5):255-267; Hwang, et al. N- Terminal Acetylation of Cellular Proteins Creates Specific Degradation Signals. Science. 2010 February 19.327(5968): 973-977; the contents of which are each incorporated herein by reference in its entirety. According to the present disclosure, references to capsid polypeptides is not limited to either clipped (Met−/AA−) or unclipped (Met+/AA+) and, in context, also refer to independent capsid polypeptides, viral capsids comprised of a mixture of capsid proteins, and/or polynucleotide sequences (or fragments thereof) which encode, describe, produce or result in capsid polypeptides of the present disclosure. A direct reference to a “capsid polypeptide” (such as VP1, VP2 or VP3) also comprise VP capsid proteins which include a Met1/AA1 amino acid (Met+/AA+) as well as corresponding VP capsid polypeptide which lack the Met1/AA1 amino acid as a result of Met/AA-clipping (Met−/AA−). Further according to the present disclosure, a reference to a specific SEQ ID NO: (whether a protein or nucleic acid) which comprises or encodes, respectively, one or more capsid polypeptides which include a Met1/AA1 amino acid (Met+/AA+) should be understood to teach the VP capsid polypeptides which lack the Met1/AA1 amino acid as upon review of the sequence, it is readily apparent any sequence which merely lacks the first listed amino acid (whether or not Met1/AA1). As a non-limiting example, reference to a VP1 polypeptide sequence which is 736 amino acids in length and which includes a “Met1” amino acid (Met+) encoded by the AUG/ATG start codon is also understood to teach a VP1 polypeptide sequence which is 735 amino acids in length and which does not include the “Met1” amino acid (Met−) of the 736 amino acid Met+sequence. As a second non-limiting example, reference to a VP1 polypeptide sequence which is 736 amino acids in length and which includes an “AA1” amino acid (AA1+) encoded by any NNN initiator codon can also be understood to teach a VP1 polypeptide sequence which is 735 amino acids in length and which does not include the “AA1” amino acid (AA1−) of the 736 amino acid AA1+sequence. References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−). As a non-limiting example, an AAV capsid serotype can include VP1 (Met+/AA1+), VP1 (Met−/AA1−), or a combination of VP1 (Met+/AA1+) and VP1 (Met- /AA1-). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

In some embodiments, the reference AAV capsid sequence comprises an amino acid sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of the those described above.

In some embodiments, the reference AAV capsid sequence is encoded by a nucleotide sequence with 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any of those described above. In certain embodiments, the reference sequence is not an AAV capsid sequence and is instead a different vector (e.g., lentivirus, plasmid, etc.).

In some embodiments, a nucleic acid of the disclosure (e.g., encoding a variant capsid polypeptide described herein) comprises conventional control elements or sequences which are operably linked to the nucleic acid molecule in a manner which permits transcription, translation and/or expression in a cell transfected with the nucleic acid (e.g., a plasmid vector comprising said nucleic acid) or infected with a virus comprising said nucleic acid. As used herein, “operably linked” sequences include both expression control sequences that are contiguous with the gene of interest and expression control sequences that act in trans or at a distance to control the gene of interest.

Expression control sequences include efficient RNA processing signals such as splicing and polyadenylation (polyA) signals; appropriate transcription initiation, termination, promoter and enhancer sequences; sequences that stabilize cytoplasmic mRNA; sequences that enhance protein stability; sequences that enhance translation efficiency (e.g., Kozak consensus sequence); and in some embodiments, sequences that enhance secretion of the encoded transgene product. Expression control sequences, including promoters which are native, constitutive, inducible and/or tissue-specific, are known in the art and may be utilized with the compositions and methods disclosed herein.

In some embodiments, the native promoter for the transgene may be used. Without wishing to be bound by theory, the native promoter may mimic native expression of the transgene, or provide temporal, developmental, or tissue-specific expression, or expression in response to specific transcriptional stimuli. In some embodiment, the transgene may be operably linked to other native expression control elements, such as enhancer elements, polyadenylation sites or Kozak consensus sequences, e.g., to mimic the native expression.

In some embodiments, the transgene is operably linked to a tissue-specific promoter.

In some embodiments, a vector, e.g., a plasmid, carrying a transgene may also include a selectable marker or a reporter gene. Such selectable reporters or marker genes can be used to signal the presence of the vector, e.g., plasmid, in bacterial cells. Other components of the vector, e.g., plasmid, may include an origin of replication. Selection of these and other promoters and vector elements are conventional and many such sequences are available (see, e.g., Sambrook et al, and references cited therein).

In some embodiments, the capsid polypeptide present in a viral particle increases transduction in the eye as compared to a viral particle with a reference capsid polypeptide, for example, with the wild-type capsid polypeptide (SEQ ID NO: 1). In some embodiments, the capsid polypeptide present in a viral particle increases transduction in the retina as compared to a viral particle with a reference capsid polypeptide, for example, with the wild-type capsid polypeptide (SEQ ID NO: 1). In some embodiments, the capsid polypeptide present in a viral particle increases transduction in the non-macular retina relative to macula and trabecular meshwork as compared to a viral particle with a reference capsid polypeptide, for example, with the wild-type capsid polypeptide (SEQ ID NO: 1). In some embodiments, the viral particle is administered intravenously.

In some embodiments, the capsid polypeptide present in a viral particle increases ocular transduction at least 1-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction at least 2-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 4-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 6-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 8-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 10-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 15-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 16-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 32-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 64-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 100-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 150-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 200-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 500-fold, e.g., as compared to a viral particle with a reference capsid polypeptide, for example, with the wild-type capsid polypeptide (SEQ ID NO: 1). In some embodiments, the capsid polypeptide present in a viral particle increases ocular transduction 1000-fold, e.g., as compared to a viral particle with a reference capsid polypeptide, for example, with the wild-type capsid polypeptide (SEQ ID NO: 1). In embodiments, increased ocular transduction is measured by comparing the level of mRNA in the target tissue (e.g., in a cell or population of cells of the target tissue) produced from a nucleic acid packaged in the variant viral particle with the level of mRNA in the target tissue (e.g., in a cell or population of cells of the target tissue) produced from a nucleic acid packaged in a reference viral particle (e.g., packaged in a capsid comprising capsid polypeptides of SEQ ID NO: 1).

In some embodiments, the capsid polypeptide is an isolated or purified polypeptide (e.g., isolated or purified from a cell, other biological component, or contaminant). In some embodiments, the variant polypeptide is present in a dependoparvovirus particle, e.g., described herein. In some embodiments, the variant capsid polypeptide is present in a cell, cell-free system, or translation system, e.g., described herein.

In some embodiments, the capsid polypeptide is present in a dependoparvovirus B (e.g., AAV2) particle. In some embodiments, the capsid particle has increased ocular transduction. In some embodiments, a dependoparvovirus particle comprises an amino acid sequence that has at least 80, 85, 90, 91, 92, 93, 94, 95, 96, 97, 98, 99, or 100% identity to the amino acid sequences provided for herein (e.g., SEQ ID NO: 2-4). In some embodiments, the variant capsid polypeptide comprises an amino acid sequence that differs by no more than 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 amino acids from the amino acid sequence of a variant capsid polypeptide provided for herein.

In some embodiments, the additional alteration improves a production characteristic of a dependoparvovirus particle or method of making the same. In some embodiments, the additional alteration improves or alters another characteristic of a dependoparvovirus particle, e.g., tropism.

### VP1 Nucleic Acids and Polypeptides

The disclosure is further directed, in part, to a nucleic acid comprising a sequence encoding a dependoparvovirus (e.g., dependoparvovirus B, e.g., an AAV2) polypeptide as provided for herein, as well as to a VP1 polypeptide encoded by the same. In some embodiments, the polypeptide comprises a sequence of SEQ ID NOs: 2, 3, or 4.

### Dependoparvovirus Particles

The disclosure is also directed, in part, to a dependoparvovirus particle (e.g., a functional dependoparvovirus particle) comprising a nucleic acid or variant capsid polypeptide described herein or produced by a method described herein.

Dependoparvovirus is a single-stranded DNA parvovirus that grows only in cells in which certain functions are provided, e.g., by a co-infecting helper virus. Several species of dependoparvovirus are known, including dependoparvovirus A and dependoparvovirus B, which include serotypes known in the art as adeno-associated viruses (AAV). At least thirteen serotypes of AAV that have been characterized. General information and reviews of AAV can be found in, for example, Carter, Handbook of Parvoviruses, Vol. 1, pp. 169-228 (1989), and Berns, Virology, pp. 1743-1764, Raven Press, (New York, 1990). AAV serotypes, and to a degree, dependoparvovirus species, are significantly interrelated structurally and functionally. (See, for example, Blacklowe, pp. 165-174 of Parvoviruses and Human Disease, J. R. Pattison, ed. (1988); and Rose, Comprehensive Virology 3:1-61 (1974)). For example, all AAV serotypes apparently exhibit very similar replication properties mediated by homologous rep genes; and all bear three related capsid proteins. In addition, heteroduplex analysis reveals extensive cross-hybridization between serotypes along the length of the genome, further suggesting interrelatedness. Dependoparvoviruses genomes also comprise self-annealing segments at the termini that correspond to “inverted terminal repeat sequences” (ITRs).

The genomic organization of naturally occurring dependoparvoviruses, e.g., AAV serotypes, is very similar. For example, the genome of AAV is a linear, single-stranded DNA molecule that is approximately 5,000 nucleotides (nt) in length or less. Inverted terminal repeats (ITRs) flank the unique coding nucleotide sequences for the non-structural replication (Rep) proteins and the structural capsid (Cap) proteins. Three different viral particle (VP) proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins: Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter.

In some embodiments, a dependoparvovirus particle of the disclosure comprises a nucleic acid comprising a capsid polypeptide provided for herein. In some embodiments, the particle comprises a variant capsid polypeptide as provided for herein.

In some embodiments, the dependoparvovirus particle of the disclosure may be an AAV2 particle or variant thereof. In some embodiments, the AAV2 particle comprises a capsid polypeptide as provided for herein and/or a nucleic acid molecule encoding the same.

In some embodiments the dependoparvovirus particle comprises a capsid comprising a variant capsid polypeptide described herein. In embodiments, the dependoparvovirus particle comprises variant capsid polypeptide described herein and a nucleic acid molecule. In embodiments, the dependoparvovirus particle comprises variant capsid polypeptide described herein and a nucleic acid molecule comprising one or more inverted terminal repeat sequences (ITRs), for example, ITRs derived from an AAV2 dependoparvovirus, one or more regulatory elements (for example, a promoter), and a payload (e.g., as described herein). In embodiments, at least one of the ITRs is modified. In embodiments, the nucleic acid molecule is single-stranded.

In embodiments, the nucleic acid molecule is self-complementary.

Increased Ocular Transduction Characteristics (Ocular Targeting) The disclosure is directed, in part, to nucleic acids, polypeptides, cells, cell free systems, translation systems, viral particles, compositions and methods associated with making the same to produce virus particles that have increased ocular transduction, e.g., retina transduction, as compared to a virus particle having capsid polypeptides of a reference sequence, e.g., with a wild-type sequence of SEQ ID NO: 1. In embodiments, such increased ocular transduction is exhibited after intravenous administration of the virus particle or composition thereof. In some embodiments, a use of a viral particle comprising the variant capsid polypeptide leads to increased ocular transduction of a transgene in the eye, and, therefore, expression of the transgene in the eye. In some embodiments, a use of a viral particle comprising the variant capsid polypeptide leads to increased ocular transduction of a transgene in the retina, and, therefore, expression of the transgene in the retina. In some embodiments, a use of a viral particle comprising the variant capsid polypeptide leads to increased ocular transduction of a transgene in the non-macular retina, and, therefore, expression of the transgene in the non-macular retina. In some embodiments, a use of a viral particle comprising the variant capsid polypeptide leads to increased ocular transduction of a transgene in the macula, and, therefore, expression of the transgene in the macula. In some embodiments, a use of a viral particle comprising the variant capsid polypeptide leads to increased ocular transduction of a transgene in the trabecular meshwork, and, therefore, expression of the transgene in the trabecular meshwork. In embodiments, the increased ocular transduction is achieved upon intravenous administration.

In some embodiments, a use of a viral particle comprising the variant capsid polypeptide leads to increased ocular transduction of a transgene in the front third of the eye, which includes the structures in front of the vitreous humor. Examples of structures in front of the vitreous humor, include the cornea, iris, ciliary body, lens, trabecular meshwork, and Schlemm's canal. Accordingly, in some embodiments, use of a viral particle comprising the variant capsid polypeptide leads to increased ocular transduction of a transgene in the cornea, iris, ciliary body, lens, trabecular meshwork, or Schlemm's canal, or any combination thereof. In some embodiments, a use of a viral particle comprising the variant capsid polypeptide leads to increased ocular transduction of a transgene posterior to the lens, such as in the anterior hyaloid membrane and all of the optical structures behind it, such as the vitreous humor, retina, choroid or optic nerve, or any combination thereof. Accordingly, in some embodiments, use of a viral particle comprising the variant capsid polypeptide leads to increased ocular transduction of a transgene in the anterior hyaloid membrane and all of the optical structures behind it, such as the vitreous humor, retina, choroid or optic nerve, or any combination thereof. In some embodiments, a use of a viral particle comprising the variant capsid polypeptide leads to increased ocular transduction of a transgene in the front third of the eye and posterior to the lens.

In some embodiments, the increase in ocular transduction is, on a log 2 scale, about 1-10 times better (e.g., about 2-5 times better, e.g., about 3-5 times better, e.g., about 7-9 times better, e.g., about 8 times better) than a virus particle having a reference sequence capsid polypeptide, e.g., having the wild-type capsid polypeptide SEQ ID NO: 1. For example, a virus particle that is 8 times better than another virus particle on a log 2 scale for transduction thus exhibits a 256-fold improvement in transduction relative to the virus particle having the reference sequence. In embodiments, the increased transduction is achieved upon intravenous administration.

In some embodiments, the capsid polypeptide present in a viral particle increases transduction without increasing the biodistribution of the variant capsid polypeptide in the eye relative to SEQ ID NO: 1. In some embodiments, the capsid polypeptide present in a viral particle increases transduction without increasing the biodistribution of the variant capsid polypeptide in the retina relative to SEQ ID NO: 1. In some embodiments, the capsid polypeptide present in a viral particle increases transduction without increasing the biodistribution of the variant capsid polypeptide in the trabecular meshwork relative to SEQ ID NO: 1. In embodiments, the capsid polypeptide present in a viral particle increases transduction in one or more regions of the eye (e.g., choroid, retina, macula, non-macular retina or trabecular meshwork) but decreases transduction in the liver, in each case relative to virus particles comprising wild-type AAV2 and/or wild-type AAV5 capsid polypeptides. In embodiments, the decrease in liver transduction is at least about 4-fold relative to either AAV2 or AAV5, and in embodiments, at least about 16-fold lower than virus particles comprising wild-type AAV2 capsid polypeptides. Relative transduction values (log 2 relative to virus particles comprising wild-type AAV2 capsid polypeptides) for virus particles comprising exemplary variant capsid polypeptides of the present invention are provided for in Table 1 and Tables 4-6. In embodiments, including in any of the aforementioned embodiments, transduction is as measured by quantitative NGS sequencing of viral RNA isolated from cells of the tissue of interest, for example as described in Example 1. In embodiments, including in any of the aforementioned embodiments, transduction is as measured by quantitative NGS sequencing of viral DNA isolated from the tissue of interest, for example as described in Example 1. In some embodiments, the capsid polypeptide present in a viral particle increases transduction without increasing the biodistribution of the variant capsid polypeptide in the eye relative to SEQ ID NO: 1. In some embodiments, the capsid polypeptide present in a viral particle increases transduction without increasing the biodistribution of the variant capsid polypeptide in the retina relative to SEQ ID NO: 1. In some embodiments, the capsid polypeptide present in a viral particle increases transduction without increasing the biodistribution of the variant capsid polypeptide in the trabecular meshwork relative to SEQ ID NO: 1.

### Increased CNS Transduction Characteristics (CNS Targeting and Dual CNS/Ocular Targeting)

The disclosure is directed, in part, to nucleic acids, polypeptides, cells, cell free systems, translation systems, viral particles, compositions and methods associated with making the same to produce virus particles that have increased central nervous system (“CNS”) transduction (e.g., whole-brain, cerebellum and/or midbrain) transduction, as compared to a virus particle having capsid polypeptides of a reference sequence, e.g., with a wild-type sequence of SEQ ID NO: 1. In embodiments, such increased CNS transduction is exhibited after intravenous administration of the virus particle or composition thereof. In some embodiments, a use of a viral particle comprising the variant capsid polypeptide leads to increased transduction of a transgene in one or more regions of the brain, and, therefore, expression of the transgene in such one or more regions of the brain. In some embodiments, a use of a viral particle comprising the variant capsid polypeptide leads to increased transduction of a transgene in the midbrain, and, therefore, expression of the transgene in the midbrain. In some embodiments, a use of a viral particle comprising the variant capsid polypeptide leads to increased transduction of a transgene in the cerebellum, and, therefore, expression of the transgene in the cerebellum. In some embodiments, a use of a viral particle comprising the variant capsid polypeptide leads to increased transduction of a transgene in one or more regions of the CNS (e.g., cerebellum and/or midbrain) and leads to increased transduction of a transgene in one or more regions of the eye (e.g., retina, macula, choroid and/or trabecular meshwork), and, therefore, expression of the transgene in such combination of tissues. In embodiments, the increased CNS or CNS and ocular transduction is achieved upon intravenous administration.

In some embodiments, the increase in CNS transduction is, on a log 2 scale, about 1-10 times better (e.g., about 2-8 times better, e.g., about 3-8 times better, e.g., about 5-8 times better, e.g., about or at least 8 times better) than a virus particle having a reference sequence capsid polypeptide, e.g., having the wild-type capsid polypeptide SEQ ID NO: 1. For example, a virus particle that is 8 times better than another virus particle on a log 2 scale for transduction thus exhibits a 256-fold improvement in transduction relative to the virus particle having the reference sequence. In embodiments, the increased transduction is achieved upon intravenous administration.

### Decreased Liver Transduction Characteristics (Liver Detargeting)

In embodiments, the capsid polypeptide present in a viral particle increases transduction in one or more regions of the eye (e.g., choroid, retina, macula, non-macular retina or trabecular meshwork) and/or one or more regions of the CNS, but decreases transduction in the liver, in each case relative to virus particles comprising wild-type AAV2 and/or wild-type AAV5 capsid polypeptides. In embodiments, the decrease in liver transduction is at least about 4-fold relative to either wild-type AAV2 or wild-type AAV5, and in embodiments, at least about 16-fold lower than virus particles comprising wild-type AAV2 capsid polypeptides.

In embodiments, provided herein are capsid polypeptide and a virus particle comprising said capsid polypeptide, as described herein, wherein the virus particle comprising said capsid polypeptide (as described herein) exhibits (1) increased transduction in one or more ocular tissues (e.g., neural retina, neural retina layer of the macular and/or retina, choroid and/or trabecular meshwork), (2) increased transduction in one or more CNS tissues (e.g., midbrain and/or cerebellum), and (3) decreased transduction in one or more liver tissues, relative to virus particles comprising capsid polypeptides of wild-type AAV2 (e.g., capsid polypeptides of SEQ ID NO: 1). In embodiments, the increased transduction to one or more ocular tissues is at least 10-fold, at least 20-fold, at least 50-fold or at least 100-fold the level of transduction exhibited by an otherwise identical virus particle having wild-type AAV2 capsid polypeptides (e.g., capsid polypeptides of SEQ ID NO: 1); the increased transduction to one or more CNS tissues is at least 20-fold, at least 40 fold, at least 100-fold, at least 150-fold or at least 300-fold the level of transduction exhibited by an otherwise identical virus particle having wild-type AAV2 capsid polypeptides (e.g., capsid polypeptides of SEQ ID NO: 1); and the decreased transduction to one or more liver tissues is no greater than 25% or no greater than 5% the level of transduction exhibited by an otherwise identical virus particle having wild-type AAV2 capsid polypeptides (e.g., capsid polypeptides of SEQ ID NO: 1). In embodiments, the transduction is measured after systemic (e.g., intravenous) administration, e.g., to a mammal, e.g., to a non-human primate. In embodiments, the transduction is as measured by quantitative NGS sequencing of viral RNA isolated from the tissue of interest, e.g., as described in Example 1.

Relative transduction values (log 2 relative to virus particles comprising wild-type AAV2 (and in the case of liver, wild-type AAV5) capsid polypeptides) for virus particles comprising exemplary variant capsid polypeptides of the present invention are provided for in Table 1 and Tables 4-7. In embodiments, including in any of the aforementioned embodiments, transduction is as measured by quantitative NGS sequencing of viral RNA isolated from cells of the tissue of interest, for example as described in Example 1. In embodiments, including in any of the aforementioned embodiments, biodistribution is as measured by quantitative NGS sequencing of viral DNA isolated from the tissue of interest, for example as described in Example 1.

### Methods of Making Compositions Described Herein

The disclosure is directed, in part, to a method of making a capsid polypeptide described herein or a dependoparvovirus particle, e.g., a dependoparvovirus particle described herein. In some embodiments, a method of making dependoparvovirus particle comprises providing a cell, cell-free system, or other translation system, comprising a nucleic acid described herein encoding a variant capsid polypeptide provided for herein, or a polypeptide provided for herein (e.g., a variant capsid polypeptide); and cultivating the cell, cell-free system, or other translation system under conditions suitable for the production of the dependoparvovirus particle, thereby making the dependoparvovirus particle.

In some embodiments, providing a cell comprising a nucleic acid described herein comprises introducing the nucleic acid to the cell, e.g., transfecting or transforming the cell with the nucleic acid. The nucleic acids of the disclosure may be situated as a part of any genetic element (vector) which may be delivered to a host cell, e.g., naked DNA, a plasmid, phage, transposon, cosmid, episome, a protein in a non-viral delivery vehicle (e.g., a lipid-based carrier), virus, etc. which transfer the sequences carried thereon. Such a vector may be delivered by any suitable method, including transfection, liposome delivery, electroporation, membrane fusion techniques, viral infection, high velocity DNA- coated pellets, and protoplast fusion. A person of skill in the art possesses the knowledge and skill in nucleic acid manipulation to construct any embodiment of this invention and said skills include genetic engineering, recombinant engineering, and synthetic techniques. See, e.g., Sambrook et al, Molecular Cloning: A Laboratory Manual, Cold Spring Harbor Press, Cold Spring Harbor, NY.

In some embodiments, a vector of the disclosure comprises sequences encoding a dependoparvovirus variant capsid polypeptide as provided for herein or a fragment thereof. In some embodiments, vectors of the disclosure comprises sequences encoding a dependoparvovirus rep protein or a fragment thereof. In some embodiments, such vectors may contain sequence encoding both dependoparvovirus cap (e.g., a variant capsid polypeptide described herein) and rep proteins. In vectors in which both AAV rep and cap are provided, the dependoparvovirus rep and dependoparvovirus cap sequences may both be of the same dependoparvovirus species or serotype origin, such as AAV2. Alternatively, the present disclosure also provides vectors in which the rep sequences are from a dependoparvovirus species or serotype which differs from that from which the cap sequences are dervied. In some embodiments, the rep and cap sequences are expressed from separate sources (e.g., separate vectors, or a host cell genome and a vector). In some embodiments, the rep sequences are fused in frame to cap sequences of a different dependoparvovirus species or serotype to form a chimeric dependoparvovirus vector. In some embodiments, the vectors of the invention further contain a payload, e.g., a minigene comprising a selected transgene (e.g., a payload as described herein), e.g., flanked by dependoparvovirus 5′ ITR and dependoparvovirus 3′ ITR. The vectors described herein, e.g., a plasmid, are useful for a variety of purposes, but are particularly well suited for use in production of recombinant dependoparvovirus particles comprising dependoparvovirus sequences or a fragment thereof, and in some embodiments, a payload.

In some embodiments, the disclosure provides a method of making a dependoparvovirus particle (e.g., a dependoparvovirus B particle, e.g., an AAV2 particle or particle comprising a variant capsid polypeptide as described herein), or a portion thereof. In some embodiments, the method comprises culturing a host cell which contains a nucleic acid sequence encoding a dependoparvovirus variant capsid polypeptide as provided for herein, or fragment thereof, ; a functional rep gene; a payload (e.g., as described herein), e.g., a minigene comprising dependoparvovirus inverted terminal repeats (ITRs) and a transgene, optionally under the control of a regulatory element such as a promoter; and sufficient helper functions to promote packaging of the payload, e.g., minigene, into the dependoparvovirus capsid. The components necessary to be cultured in the host cell to package a payload, e.g., minigene, in a dependoparvovirus capsid may be provided to the host cell in trans. In some embodiments, any one or more of the required components (e.g., payload (e.g., minigene), rep sequences, cap sequences, and/or helper functions) may be provided by a host cell which has been engineered to stably comprise one or more of the required components using methods known to those of skill in the art. In some embodiments, a host cell which has been engineered to stably comprise the required component(s) comprises it under the control of an inducible promoter. In some embodiments, the required component may be under the control of a constitutive promoter. Examples of suitable inducible and constitutive promoters are provided herein and further examples are known to those of skill in the art. In some embodiments, a selected host cell which has been engineered to stably comprise one or more components may comprise a component under the control of a constitutive promoter and another component under the control of one or more inducible promoters. For example, a host cell which has been engineered to stably comprise the required components may be generated from 293 cells (e.g., which comprise helper functions under the control of a constitutive promoter), which comprises the rep and/or cap proteins under the control of one or more inducible promoters.

The payload (e.g., minigene), rep sequences, cap sequences, and helper functions required for producing a dependoparvovirus particle of the disclosure may be delivered to the packaging host cell in the form of any genetic element which transfers the sequences carried thereon (e.g., in a vector or combination of vectors). The genetic element may be delivered by any suitable method, including those described herein. Methods used to construct genetic elements, vectors, and other nucleic acids of the disclosure are known to those with skill and include genetic engineering, recombinant engineering, and synthetic techniques. See, e.g., Sambrook et al, Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al, J. Virol, 70:520-532 (1993) and U.S. Pat. No. 5,478,745. Unless otherwise specified, the dependoparvovirus ITRs, and other selected dependoparvovirus components described herein, may be readily selected from among any dependoparvovirus species and serotypes, e.g., AAV1, AAV2, AAV3, AAV4, AAV5, AAV6, AAV7, AAV9. ITRs or other dependoparvovirus components may be readily isolated using techniques available to those of skill in the art from a dependoparvovirus species or serotype. Dependoparvovirus species and serotypes may be isolated or obtained from academic, commercial, or public sources (e.g., the American Type Culture Collection, Manassas, VA). In some embodiments, the dependoparvovirus sequences may be obtained through synthetic or other suitable means by reference to published sequences such as are available in the literature or in databases such as, e.g., GenBank or PubMed.

The dependoparvovirus particles (e.g., including a variant capsid polypeptide and, for example, a payload) of the disclosure may be produced using any invertebrate cell type which allows for production of dependoparvovirus or biologic products and which can be maintained in culture. In some embodiments, an insect cell may be used in production of the compositions described herein or in the methods of making a dependoparvovirus particle described herein. For example, an insect cell line used can be from Spodoptera frugiperda, such as Sf9, SF21, SF900+, drosophila cell lines, mosquito cell lines, e.g., Aedes albopictus derived cell lines, domestic silkworm cell lines, e.g. Bombyxmori cell lines, Trichoplusia ni cell lines such as High Five cells or Lepidoptera cell lines such as Ascalapha odorata cell lines. In some embodiments, the insect cells are susceptible to baculovirus infection, including High Five, Sf9, Se30l, SeIZD2109, SeUCR1, SP900+, Sf21, BTI-TN-5B1-4, MG-1, Tn368, HzAml, BM-N, Ha2302, Hz2E5 and Ao38.

In some embodiments, the methods of the disclosure can be carried out with any mammalian cell type which allows for replication of dependoparvovirus or production of biologic products, and which can be maintained in culture. In some embodiments, the mammalian cells used can be HEK293, HEK293T, HeLa, CHO, NSO, SP2/0, PER.C6, Vero, RD, BHK, HT 1080, A549, Cos-7, ARPE-19 or MRC-5 cells. In some embodiments the culture is an adherent cell culture. In some embodiments, the culture is a suspension cell culture.

Methods of expressing proteins (e.g., recombinant or heterologous proteins, e.g., dependoparvovirus polypeptides) in insect cells are well documented, as are methods of introducing nucleic acids, such as vectors, e.g., insect-cell compatible vectors, into such cells and methods of maintaining such cells in culture. See, for example, METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. 63:3822-8 (1989); Kajigaya et al., Proc. Nat'l. Acad. Sci. USA 88:4646-50 (1991); Ruffing et al., J. Vir. 66:6922-30 (1992); Kirnbauer et al., Vir. 219:37-44 (1996); Zhao et al., Vir. 272:382-93 (2000); and Samulski et al., U.S. Pat. No. 6,204,059. In some embodiments, a nucleic acid construct encoding dependoparvovirus polypeptides (e.g., a dependoparvovirus genome) in insect cells is an insect cell-compatible vector. An “insect cell-compatible vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cell's genome or remain present extra-chromosomally. The vector may be present permanently or transiently, e.g., as an episomal vector. Vectors may be introduced by any means known in the art. Such means include but are not limited to chemical treatment of the cells, electroporation, or infection. In some embodiments, the vector is a baculovirus, a viral vector, or a plasmid.

In some embodiments, a nucleic acid sequence encoding an dependoparvovirus polypeptide is operably linked to regulatory expression control sequences for expression in a specific cell type, such as Sf9 or HEK cells. Techniques known to one skilled in the art for expressing foreign genes in insect host cells or mammalian host cells can be used with the compositions and methods of the disclosure. Methods for molecular engineering and expression of polypeptides in insect cells is described, for example, in Summers and Smith. A Manual of Methods for Baculovirus Vectors and Insect Culture Procedures, Texas Agricultural Experimental Station Bull. No. 7555, College Station, Tex. (1986); Luckow. 1991. In Prokop et al., Cloning and Expression of Heterologous Genes in Insect Cells with Baculovirus Vectors' Recombinant DNA Technology and Applications, 97-152 (1986); King, L. A. and R. D. Possee, The baculovirus expression system, Chapman and Hall, United Kingdom (1992); O'Reilly, D. R., L. K. Miller, V. A. Luckow, Baculovirus Expression Vectors: A Laboratory Manual, New York (1992); W. H. Freeman and Richardson, C. D., Baculovirus Expression Protocols, Methods in Molecular Biology, volume 39 (1995); U.S. Pat. No. 4,745,051; US2003148506; and WO 03/074714. Promoters suitable for transcription of a nucleotide sequence encoding a dependoparvovirus polypeptide include the polyhedron, , p10, p35 or IE-1 promoters and further promoters described in the above references are also contemplated.

In some embodiments, providing a cell comprising a nucleic acid described herein comprises acquiring a cell comprising the nucleic acid.

Methods of cultivating cells, cell-free systems, and other translation systems are known to those of skill in the art. In some embodiments, cultivating a cell comprises providing the cell with suitable media and incubating the cell and media for a time suitable to achieve viral particle production.

In some embodiments, a method of making a dependoparvovirus particle further comprises a purification step comprising isolating the dependoparvovirus particle from one or more other components (e.g., from a cell or media component).

In some embodiments, production of the dependoparvovirus particle comprises one or more (e.g., all) of: expression of dependoparvovirus polypeptides, assembly of a dependoparvovirus capsid (e.g., a capsid comprising a variant capsid polypeptide provided for herein), expression (e.g., duplication) of a dependoparvovirus genome, and packaging of the dependoparvovirus genome into the dependoparvovirus capsid to produce a dependoparvovirus particle. In some embodiments, production of the dependoparvovirus particle further comprises secretion of the dependoparvovirus particle.

In some embodiments, and as described elsewhere herein, the nucleic acid molecule encoding the variant capsid polypeptide is disposed in a dependoparvovirus genome. In some embodiments, and as described elsewhere herein, the nucleic acid molecule encoding the variant capsid polypeptide is packaged into a dependoparvovirus particle along with the dependoparvovirus genome as part of a method of making a dependoparvovirus particle described herein. In other embodiments, the nucleic acid molecule encoding the variant capsid polypeptide is not packaged into a dependoparvovirus particle made by a method described herein.

In some embodiments, a method of making a dependoparvovirus particle described herein produces a dependoparvovirus particle comprising a payload (e.g., a payload described herein) and the variant capsid polypeptide. In some embodiments, the payload comprises a second nucleic acid (e.g., in addition to the dependoparvovirus genome), and production of the dependoparvovirus particle comprises packaging the second nucleic acid into the dependoparvovirus particle. In some embodiments, a cell, cell-free system, or other translation system for use in a method of making a dependoparvovirus particle comprises the second nucleic acid. In some embodiments, the second nucleic acid comprises an exogenous sequence (e.g., exogenous to the dependoparvovirus, the cell, or to a target cell or subject who will be administered the dependoparvovirus particle). In some embodiments, the exogenous sequence encodes an exogenous polypeptide. In some embodiments, the exogenous sequence encodes a therapeutic product.

In some embodiments, a nucleic acid or polypeptide described herein is produced by a method known to one of skill in the art. The nucleic acids, polypeptides, and fragments thereof of the disclosure may be produced by any suitable means, including recombinant production, chemical synthesis, or other synthetic means. Such production methods are within the knowledge of those of skill in the art and are not a limitation of the present invention.

### Applications

The disclosure is directed, in part, to compositions comprising a nucleic acid, polypeptide, or particles described herein. The disclosure is further directed, in part, to methods utilizing a composition, nucleic acid, polypeptide, or particles described herein. As will be apparent based on the disclosure, nucleic acids, polypeptides, particles, and methods disclosed herein have a variety of utilities.

The disclosure is directed, in part, to a vector comprising a nucleic acid described herein, e.g., a nucleic acid encoding a variant capsid polypeptide. Many types of vectors are known to those of skill in the art. In some embodiments, a vector comprises a plasmid. In some embodiments, the vector is an isolated vector, e.g., removed from a cell or other biological components.

The disclosure is directed, in part to a cell, cell-free system, or other translation system, comprising a nucleic acid or vector described herein, e.g., a nucleic acid or vector comprising a nucleic acid molecule encoding a variant capsid polypeptide. In some embodiments, the cell, cell-free system, or other translation system is capable of producing dependoparvovirus particles comprising the variant capsid polypeptides. In some embodiments, the cell, cell-free system, or other translation system comprises a nucleic acid comprising a dependoparvovirus genome or components of a dependoparvovirus genome sufficient to promote production of dependoparvovirus particles comprising the variant capsid polypeptides.

In some embodiments, the cell, cell-free system, or other translation system further comprises one or more non-dependoparvovirus nucleic acid sequences that promote dependoparvovirus particle production and/or secretion. Said sequences are referred to herein as helper sequences. In some embodiments, a helper sequence comprises one or more genes from another virus, e.g., an adenovirus or herpes virus. In some embodiments, the presence of a helper sequence is necessary for production and/or secretion of a dependoparvovirus particle. In some embodiments, a cell, cell-free system, or other translation system comprises a vector, e.g., plasmid, comprising one or more helper sequences.

In some embodiments, a cell, cell-free system, or other translation system comprises a first nucleic acid and a second nucleic acid, wherein the first nucleic acid comprises a sequences encoding one or more dependoparvovirus genes (e.g., a Cap gene, a Rep gene, or a complete dependoparvovirus genome) and a helper sequence, and wherein the second nucleic acid comprises a payload. In some embodiments, a cell, cell-free system, or other translation system comprises a first nucleic acid and a second nucleic acid, wherein the first nucleic acid comprises a sequences encoding one or more dependoparvovirus genes (e.g., a Cap gene, a Rep gene, or a complete dependoparvovirus genome) and a payload, and wherein the second nucleic acid comprises a helper sequence. In some embodiments, a cell, cell-free system, or other translation system comprises a first nucleic acid and a second nucleic acid, wherein the first nucleic acid comprises a helper sequence and a payload, and wherein the second nucleic acid comprises a sequences encoding one or more dependoparvovirus genes (e.g., a Cap gene, a Rep gene, or a complete dependoparvovirus genome). In some embodiments, a cell, cell-free system, or other translation system comprises a first nucleic acid, a second nucleic acid, and a third nucleic acid, wherein the first nucleic acid comprises a sequences encoding one or more dependoparvovirus genes (e.g., a Cap gene, a Rep gene, or a complete dependoparvovirus genome), the second nucleic acid comprises a helper sequence, and the third nucleic acid comprises a payload.

In some embodiments, the first nucleic acid, second nucleic acid, and optionally third nucleic acid are situated in separate molecules, e.g., separate vectors or a vector and genomic DNA. In some embodiments, one, two, or all of the first nucleic acid, second nucleic acid, and optionally third nucleic acid are integrated (e.g., stably integrated) into the genome of a cell.

A cell of the disclosure may be generated by transfecting a suitable cell with a nucleic acid described herein. In some embodiments, a method of making a dependoparvovirus particle comprising a variant capsid polypeptide as provided for herein or improving a method of making a dependoparvovirus particle comprises providing a cell described herein. In some embodiments, providing a cell comprises transfecting a suitable cell with one or more nucleic acids described herein.

In some embodiments, the virus particle comprising the variant capsid is produced at a level at least 10%, at least 20%, at least 50%, or at least 100% of the production level of wt AAV2 from the same producer cell type, e.g., from HEK293 cells, e.g., from adherent culture of HEK293 cells. In some embodiments, the virus particle comprising the variant capsid is produced at a level at least 10%, at least 20%, at least 50%, at least 100%, at least 200% or greater than the production level of wt AAV2 from the same producer cell type, e.g., from HEK293 cells, e.g., from adherent culture of HEK293 cells.

Many types and kinds of cells suitable for use with the nucleic acids and vectors described herein are known in the art. In some embodiments, the cell is a human cell. In some embodiments, the cell is an immortalized cell or a cell from a cell line known in the art. In some embodiments, the cell is an HEK293 cell.

### Virus Particle and Methods of Delivering a Payload

The disclosure is directed, in part, to a method of delivering a payload to a cell, e.g., a cell in a subject or in a sample. In some embodiments, a method of delivering a payload to a cell comprises contacting the cell with a dependoparvovirus particle comprising a variant capsid polypeptide (e.g., described herein) and comprising a payload (e.g., described herein). In some embodiments, the dependoparvovirus particle is a dependoparvovirus particle described herein and comprises a payload described herein. In some embodiments, the cell is an ocular cell. In some embodiments the cell is a CNS cell. In some embodiments, the ocular cell is in the retina, macula, or trabecular meshwork. In some embodiments, the ocular cell is in the retina. In some embodiments, the ocular cell is in the macula. In some embodiments, the ocular cell is in the trabecular meshwork. In some embodiments the cell is a cerebellum cell. In some embodiments, the cell is a midbrain cell. In some embodiments, the disclosure is directed, in part, to a method of delivering a payload to a CNS cell and an ocular cell, e.g., a cell in a subject or in a sample, comprising contacting the cell with a dependoparvovirus particle comprising a variant capsid polypeptide (e.g., described herein) and comprising a payload (e.g., described herein), wherein the payload is delivered both to the CNS cell and the ocular cell. In embodiments, the payload is delivered with higher efficiency to the CNS and/or ocular cell than to other cell types, for example a liver cell.

In some embodiments, the ocular cell is in the front third of the eye, which includes the structures in front of the vitreous humor. Examples of structures in front of the vitreous humor, include the cornea, iris, ciliary body, lens, trabecular meshwork, and Schlemm's canal. Accordingly, in some embodiments, the cell is in the cornea, iris, ciliary body, lens, trabecular meshwork, or Schlemm's canal, or any combination thereof.

In some embodiments, the ocular cell is posterior to the lens, such as in the anterior hyaloid membrane and all of the optical structures behind it, such as the vitreous humor, retina, choroid or optic nerve, or any combination thereof. Accordingly, in some embodiments, the cell is in the anterior hyaloid membrane and all of the optical structures behind it, such as the vitreous humor, retina, choroid or optic nerve, or any combination thereof.

The disclosure is further directed in part to a virus particle comprising a capsid polypeptide described herein. In embodiments, the virus particle comprises a capsid polypeptide described herein and a nucleic acid expression construct. In embodiments the nucleic acid expression construct of the virus particle comprises a payload.

In some embodiments, the payload comprises a transgene. In some embodiments, the transgene is a nucleic acid sequence heterologous to the vector sequences flanking the transgene which encodes a polypeptide, RNA (e.g., a miRNA or siRNA) or other product of interest. The nucleic acid of the transgene may be operatively linked to a regulatory component in a manner sufficient to promote transgene transcription, translation, and/or expression in a host cell.

A transgene may be any polypeptide or RNA encoding sequence and the transgene selected will depend upon the use envisioned. In some embodiments, a transgene comprises a reporter sequence, which upon expression produces a detectable signal. Such reporter sequences include, without limitation, DNA sequences encoding colorimetric reporters (e.g., P-lactamase, P-galactosidase (LacZ), alkaline phosphatase), cell division reporters (e.g., thymidine kinase), fluorescent or luminescence reporters (e.g., green fluorescent protein (GFP) or luciferase), resistance conveying sequences (e.g., chloramphenicol acetyltransferase (CAT)), or membrane bound proteins including to which high affinity antibodies directed thereto exist or can be produced by conventional means, e.g., comprising an antigen tag, e.g., hemagglutinin or Myc. In some embodiments, a reporter sequence operably linked with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry.

In some embodiments, the transgene encodes a product which is useful in biology and medicine, such as RNA, proteins, peptides, enzymes, dominant negative mutants. In some embodiments, the RNA comprises a tRNA, ribosomal RNA, dsRNA, catalytic RNAs, small hairpin RNA, siRNA, trans-splicing RNA, and antisense RNAs. In some embodiments, the RNA inhibits or abolishes expression of a targeted nucleic acid sequence in a treated subject (e.g., a human or animal subject).

In some embodiments, the transgene may be used to correct or ameliorate gene deficiencies. In some embodiments, gene deficiencies include deficiencies in which normal genes are expressed at less than normal levels or deficiencies in which the functional gene product is not expressed. In some embodiments, the transgene encodes a therapeutic protein or polypeptide which is expressed in a host cell. In some embodiments, a dependoparvovirus particle may comprise or deliver multiple transgenes, e.g., to correct or ameliorate a gene defect caused by a multi-subunit protein. In some embodiments, a different transgene (e.g., each situated/delivered in a different dependoparvovirus particle, or in a single dependoparvovirus particle) may be used to encode each subunit of a protein, or to encode different peptides or proteins, e.g., when the size of the DNA encoding the protein subunit is large, e.g., for immunoglobulin, platelet-derived growth factor, or dystrophin protein. In some embodiments, different subunits of a protein may be encoded by the same transgene, e.g., a single transgene encoding each of the subunits with the DNA for each subunit separated by an internal ribozyme entry site (IRES) or enzymatically cleavable sequence (e.g., a furin cleavage site). In some embodiments, the DNA may be separated by sequences encoding a 2A peptide, which self-cleaves in a post-translational event. See, e.g., Donnelly et al, J. Gen. Virol., 78(Pt 1):13-21 (January 1997); Furler, et al, Gene Ther., 8(11):864-873 (June 2001); Klump et al., Gene Ther 8(10):811-817 (May 2001).

In some embodiments, virus particles comprising a genome are provided, wherein the genome includes a nucleic acid expression construct. The nucleic acid expression construct can include a payload, for example a payload comprising a heterologous transgene and one or more regulatory elements.

In some embodiments, the particle delivers the payload to the eye with increased transduction in one or more regions of the eye as compared to a virus particle comprising capsid polypeptides of SEQ ID NO: 1, and wherein the increase in transduction is at least 2-times, 4-times, 8-times, 16-times, 32-times, 64-times, 100-times, 150-times, 200-times, 250-times, or 500-times as compared to a virus particle comprising capsid polypeptides of SEQ ID NO: 1.

In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to non-macular retina tissue relative to macular tissue. In some embodiments, the particle delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times as compared to a virus particle comprising capsid polypeptides of SEQ ID NO: 1, and wherein the increase in transduction is specific to non-macular retina tissue relative to trabecular meshwork tissue.

In some embodiments, a virus particle described herein (e.g., comprising a variant capsid polypeptide described herein) exhibits increased retinal transduction, e.g., at least 200-times or at least 250-times increased retinal transduction relative to a virus particle comprising capsid polypeptides of SEQ ID NO: 1, for example after intravenous administration, and is produced to a level at least 10%, at least 20%, at least 50% or at least 100% the level of production relative to a virus particle comprising capsid polypeptides of SEQ ID NO: 1. In some embodiments, a virus particle described herein (e.g., comprising a variant capsid polypeptide described herein) exhibits increased retinal transduction that is at least 200-times increased retinal transduction relative to a virus particle comprising capsid polypeptides of SEQ ID NO: 1, for example after intravenous administration, and is produced to a level at least 10%, at least 20%, at least 50% or at least 100% the level of production relative to a virus particle comprising capsid polypeptides of SEQ ID NO: 1. In some embodiments, a virus particle described herein (e.g., comprising a variant capsid polypeptide described herein) exhibits increased retinal transduction that is at least 250-times increased retinal transduction relative to a virus particle comprising capsid polypeptides of SEQ ID NO: 1, for example after intravenous administration, and is produced to a level at least 10%, at least 20%, at least 50% or at least 100% the level of production relative to a virus particle comprising capsid polypeptides of SEQ ID NO: 1. In some embodiments, a virus particle described herein (e.g., comprising a variant capsid polypeptide described herein) exhibits increased retinal transduction, e.g., at least 200-times, at least 250-times or greater increased retinal transduction relative to a virus particle comprising capsid polypeptides of SEQ ID NO: 1, for example after intravenous administration, and is produced to a level at least 10%, at least 20%, at least 50% or at least 100% the level of production relative to a virus particle comprising capsid polypeptides of SEQ ID NO: 1. In some embodiments, a virus particle described herein (e.g., comprising a variant capsid polypeptide described herein) exhibits (1) increased retinal transduction, e.g., at least 200-times, at least 250-times or greater increased retinal transduction relative to a virus particle comprising capsid polypeptides of SEQ ID NO: 1, for example after intravenous administration, (2) increased CNS (e.g., midbrain and/or cerebellum) transduction, e.g., at least 20-times, at least 50-times, at least 100-times or greater increased CNS (e.g., midbrain and/or cerebellum) relative to a virus particle comprising capsid polypeptides of SEQ ID NO: 1, for example after intravenous administration, (3) and is produced to a level at least 10%, at least 20%, at least 50% or at least 100% the level of production relative to a virus particle comprising capsid polypeptides of SEQ ID NO: 1.

In some embodiments, a virus particle described herein (e.g., comprising a variant capsid polypeptide described herein) exhibits (1) increased retinal transduction, e.g., at least 200-times, at least 250-times or greater increased retinal transduction relative to a virus particle comprising capsid polypeptides of SEQ ID NO: 1, for example after intravenous administration, (2) increased CNS (e.g., midbrain and/or cerebellum) transduction, e.g., at least 20-times, at least 50-times, at least 100-times or greater increased CNS (e.g., midbrain and/or cerebellum) relative to a virus particle comprising capsid polypeptides of SEQ ID NO: 1, for example after intravenous administration, (3) decreased liver transduction, e.g., at least 3-fold, at least 10-fold or at least 30-fold decreased liver transduction relative to a virus particle comprising capsid polypeptides of SEQ ID NO: 1, for example after intravenous administration, and (4) is produced to a level at least 10%, at least 20%, at least 50% or at least 100% the level of production relative to a virus particle comprising capsid polypeptides of SEQ ID NO: 1.

In any of the aforementioned embodiments, increased transduction or biodistribution is as measured as described herein in Example 1 (for example, with respect to transduction, as measured by quantification of viral cDNA isolated from the bulk tissue, e.g., NHP tissue, of interest normalized to prevalence of that virus particle in the test article, and with respect to biodistribution, as measured by quantification of viral DNA isolated from bulk tissue, e.g., NHP tissue, of interest normalized to prevalence of that virus particle in the test article).

In some embodiments, the nucleic acid of the virus particle includes regulatory elements that include a promotor. In some embodiments, the promoter is a ubiquitous or constitutive promoter active in a mammalian cell, for example a human cell, for example, in a human cell type of interest.

In some embodiments, the cell type is an ocular cell such as, for example, a neural retinal cell, a photoreceptive retinal ganglion cell, a bipolar cell, a horizontal cell, a amacrine cell, a photoreceptor (e.g., a rod or a cone cell), an endothelial cell (e.g., a retinal pigmented epithelial cell), and endothelial-like cell, and the like. Examples of ubiquitous promoters include, but are not limited, to a CAG promoter (hybrid from a cytomegalovirus early enhancer element, a chicken-beta actin promoter, e.g., the first exon and the first intron of the chicken beta actin gene, and optionally the splice acceptor of the rabbit beta globin gene), chicken-beta actin promoter, CBA promoter, CMV promoter, human PGK promoter, ubiquitin promoter, human EF1-alpha promoter and fragments thereof. In some embodiments, the promoter is a tissue-specific promoter, for example, a promoter specific in ocular tissue or cells of the eye. Examples of ocular tissue-specific promoters include but are not limited to TBG promoters, hAAT promoters, CK8 promoters and SPc5-12 promoters, rho promoters, which are active in rods, or opsin promoters, which are active in cones. In some embodiments, the regulatory element includes a photoreceptor cell-specific regulatory element (e.g., promoter) such as, e.g., a rhodopsin promoter; a rhodopsin kinase promoter; a beta phosphodiesterase gene promoter; a retinitis pigmentosa gene promoter; an interphotoreceptor retinoid-binding protein (IRBP) gene enhancer; an IRBP gene promoter, an opsin gene promoter, a retinoschisin gene promoter, a CRX homeodomain protein gene promoter, a guanine nucleotide binding protein alpha transducing activity polypeptide 1 (GNAT1) gene promoter, a neural retina-specific leucine zipper protein (NRL) gene promoter, human cone arrestin (hCAR) promoter, and the PR2.1, PR1.7, PR1.5, and PR1.1 promoters. In some embodiments, the regulatory element includes, a retinal pigment epithelia (RPE) cell-specific regulatory element (e.g., a RPE-specific promoter), e.g., a regulatory element that confers selective expression of the operably linked gene in a RPE cell, such as, e.g., an RPE65 gene promoter, a cellular retinaldehyde-binding protein (CRALBP) gene promoter, a pigment epithelium-derived factor (PEDF aka serpin F1) gene promoter, and a vitelliform macular dystrophy (VMD2) promoter. In some embodiments, the regulatory element includes a promoter specific to a glial cell, e.g., a regulatory element that confers selective expression of the operably linked payload in a retinal glial cell, such as, e.g., a glial fibrillary acidic protein (GFAP) promoter. In some instances, the regulatory element includes a promoter that is specific to a bipolar cell (e.g., a bipolar-specific promoter), e.g., a regulatory element that confers selective expression of the operably linked payload in a bipolar cell, such as, e.g., a GRM6 promoter. In embodiments, the promoter sequence is between 100 and 1000 nucleotides in length. In embodiments, the promoter sequence is about 100, about 200, about 300, about 400, about 500, about 600, about 700, about 800, about 900 or about 1000 nucleotides in length. As used in the preceding sentence, “about” refers to a value within 50 nucleotides of the recited length. Suitable regulatory elements, e.g., promoters, may be readily selected by persons of skill in the art, such as those, but not limited to, those described herein.

In some embodiments, the nucleic acid expression construct comprises an intron. The intron may be disposed between the promoter and the heterologous transgene. In some aspects, the intron is disposed 5′ to the heterologous transgene on the expression construct, for example immediately 5′ to the heterologous transgene or 100 nucleotides or less 5′ to the heterologous transgene. In some aspects, the intron is a chimeric intron derived from human b-globin and Ig heavy chain (also known as b- globin splice donor/immunoglobulin heavy chain splice acceptor intron, or b-globin/IgG chimeric intron; Reed, R., et al. Genes and Development, 1989, incorporated herein by reference in its entirety). In other aspects, the intron is a VH4 intron or a SV40 intron.

As provided herein, in some embodiments, virus particles comprising a payload, wherein the payload includes a nucleic acid that includes a heterologous transgene are provided. In some embodiments, the heterologous transgene encodes an RNA interference agent, for example a siRNA, shRNA or other interfereing nucleic acid.

In some embodiments, the payload includes a heterologous transgene that encodes a therapeutic polypeptide. In some aspects, the heterologous transgene is a human gene or fragment thereof. In some aspects, the therapeutic polypeptide is a human protein. In some embodiments, the heterologous transgene of the virus particle encodes a molecule useful in treating a disease, and the virus particle is administered to a patient in need thereof to treat said disease. Examples of diseases (and heterologous transgenes or molecules encoded by said heterologous transgenes) according to the present disclosure include: MPSI (alpha-L-iduronidase (IDUA)); MPS II-Hunter syndrome (iduronate-2-sulfatase (IDS)); Ceroid lipofuscinosis (i.e., neuronal ceroid lipofusciones)-Batten disease (CLN1, CLN2, CLN10, CLN13, CLN5, CLN11, CLN4, CNL14, CLN3, CLN6, CLN7, CLN8, CLN12); MPS IIIa-Sanfilippo Type A syndrome (heparin sulfate sulfatase (also called N-sulfoglucosamine sulfohydrolase (SGSH)); MPS IIIB-Sanfilippo Type b syndrome (N-acetyl-alpha-D-glucosaminidase (NAGLU)); MPS VI-Maroteaux-Lamy syndrome (arylsulfatase B); MPS IV A-Morquio syndrome type A (GALNS); MPS IV B-Morquio syndrome type B (GLB1); Osteogenesis Imperfecgta Type I, II, III or IV (COL1A1 and/or COL1A2); hereditary angioedema (SERPINGI, C1NH); Osteogenesis Imperfecta Type V (IFITM5); Osteogenesis Imperfecta Type VI (SERPINFI); Osteogenesis Imperfecta Type VII (CRTAP); Osteogenesis Imperfecta Type VIII (LEPRE1 and/or P3H1); Osteogenesis Imperfecta Type IX (PPIB); Gaucher disease type I, II and III (Glucocerebrosidase; GBAl); Parkinson's Disease (Glucocerebrosidase; GBAl and/or dopamine decarboxylase); Pompe (acid maltase; GAA; hGAA); Metachromatic leukodystrophy (Aryl sulfatase A); MPS VII-Sly syndrome (beta-glucuronidase); MPS VIII (glucosamine-6-sulfate sulfatase); MPS IX (Hyaluronidase); maple syrup urine disease (BCKDHA, BCKDHB, and/or DBT); Niemann-Pick disease (Sphingomyelinase); Parkinson's disease (anti-alpha synuclein RNAi); Alzheimer's disease (anit-mutant APP RNAi); Niemann-Pick disease without sphingomyelinase deficiency (NPC1 or NPC gene encoding a cholesterol metabolizing enzyme); Tay-Sachs disease (alpha subunit of beta-hexosaminidase); Sandhoff disease (both alpha and beta subunit of beta-hexosaminidase); Fabry Disease (alpha-galactosidase); Fucosidosis (fucosidase (FUCAl)); Alpha-mannosidosis (alpha-mannosidase); Beta-mannosidosis (beta-mannosidase); Wolman disease (cholesterol ester hydrolase); Dravet syndrome (SCN1A, SCN1B, SCN2A, GABRG2); Parkinson's disease (Neurturin); Parkinson's disease (glial derived growth factor (GDGF)); Parkinson's disease (tyrosine hydroxylase); Parkinson's disease (glutamic acid decarboxylase; FGF-2; BDGF); Spinal Muscular Atrophy (SMN, including SMN1 or SMN2); Friedreich's ataxia (Frataxin); Amyotrophic lateral sclerosis (ALS) (SOD1 inhibitor, e.g., anti-SOD1 RNAi); Glycogen Storage Disease la (Glucose-6-phosphatase); XLMTM (MTMI); Crigler Najjar (UGTlAl); CPVT (CASQ2); spinocerebellar ataxia (ATXN2; ATXN3 or other ATXN gene; anti-mutant Machado-Joseph disease/SCA3 allele RNAi); Rett syndrome (MECP2 or fragment thereof); Achromatopsia (CNGB3, CNGA3, GNAT2, PDE6C); Choroidermia (CDM); Danon Disease (LAMP2); Cystic Fibrosis (CFTR or fragment thereof); Duchenne Muscular Dystrophy (Mini-/ Micro-Dystrophin Gene); SARS-Cov-2 infection (anti-SARS-Cov-2 RNAi, SARS-Cov-2 genome fragments or S protein (including variants)); Limb Girdle Muscular Dystrophy Type 2C Gamma-sarcoglycanopathy (human-alpha-sarcoglycan); Advanced Heart Failure (SERCA2a); Rheumatoid Arthritis (TNFR:Fc Fusion; anti-TNF antibody or fragment thereof); Leber Congenital Amaurosis (GAA); X-linked adrenoleukodystrophy (ABCD1); Limb Girdle Muscular Dystrophy Type 2C-Gamma-sarcoglycanopathy (gamma-sarcoglycan); Angelman syndrome (UBE3A); Retinitis Pigmentosa (hMERTK); Age-Related Macular Degeneration (sFLT01); Phelan-McDermid syndrome (SHANK3; 22q13.3 replacement); Becker Muscular Dystrophy and Sporadic Inclusion Body Myositis (huFollistatin344); Parkinson's Disease (GDNF); Metachromatic Leukodystrophy-MLD (cuARSA); Hepatitis C (anti-HCV RNAi); Limb Girdle Muscular Dystrophy Type 2D (hSGCA); Human Immunodeficiency Virus Infections; (PG9DP); Acute Intermittant Porphyria (PBGD); Leber's Hereditary Optical Neuropathy (PIND4v2); Alpha-1 Antitrypsin Deficiency (alphaIAT); X-linked Retinoschisis (RS1); Choroideremia (hCHM); Giant Axonal Neuropathy (GAN); Hemophilia B (Factor IX); Homozygous FH (hLDLR); Dysferlinopathies (DYSF); Achromatopsia (CNGA3 or CNGB3); Progressive supranuclear palsy (MAPT; anti-Tau; anti-MAPT RNAi); Omithine Transcarbamylase deficiency (OTC); Hemophilia A (Factor VIII); Age-related macular degeneration (AMD), including wetAMD (anti-VEGF antibody or RNAi); X-Linked Retinitis Pigmentosa (RPGR); Myotonic dystrophy Type 1 (DMPK; anti-DMPK RNAi, including anti-CTG trinucleotide repeat RNAi); Myotonic dystrophy Type 2 (CNBP); Facioscapulohumeral muscular dystrophy (D4Z4 DNA); oculopharynggeal muscular dystrophy (PABPN1; mutated PABPN1 inhibitor (e.g., RNAi)); Mucopolysaccharidosis Type VI (hARSB); Leber Hereditary Optic Neuropathy (ND4); X-Linked myotubular Myopathy (MTMI); Crigler-Najjar Syndrome (UGTlAl); Retinitis Pigmentosa (hPDE6B); Mucopolysaccharidosis Type 3B (hNAGLU); Duchenne Muscular Dystrophy (GALGT2); Alzheimer's Disease (NGF; ApoE4; ApoE2; ApoE3; Anti-ApoE RNAi); Familial Lipoprotein Lipase Deficiency (LPL); Alpha-1 Antitrypsin Deficiency (hAAT); Leber Congenital Amaurosis 2 (hRPE65v2); Batten Disease; Late Infantile Neuronal Lipofuscinosis (CLN2); Huntington's disease (HTT; anti-HTT RNAi); Fragile X syndrome (FMR1); Leber's Hereditary Optical Neuropathy (PlND4v2); Aromatic Amino Acid Decarboxylase Deficiency (hAADC); Retinitis Pigmentosa (hMERKTK); and Retinitis Pigmentosa (RLBP1).

In some aspects, the heterologous transgene encodes an antibody or fragment thereof (for example an antibody light chain, an antibody heavy chain, a Fab or an scFv). Examples of antibodies or fragments thereof that are encoded by the heterologous transgene include but are not limited to; and an anti-Ab antibody (e.g. solanezumab, GSK933776, and lecanemab), anti-sortilin (e.g. AL-001), anti-Tau (e.g. ABBV-8E12, UCB-0107, and NI- 105), anti-SEMA4D (e.g. VX15/2503), anti-alpha synuclein (e.g. prasinezumab, NI-202, and MED-1341), anti-SOD1 (e.g. NI-204), anti-CGRP receptor (e.g. eptinezumab, fremanezumab, or galcanezumab), anti-VEGF (e.g., sevacizumab, ranibizumab, bevacizumab, and brolucizumab), anti-EpoR (e.g., LKA-651, ), anti-ALKI (e.g., ascrinvacumab), anti-C5 (e.g., tesidolumab, ravulizumab, and eculizumab), anti-CD105 (e.g., carotuximab), anti-CClQ (e.g., ANX-007), anti-TNFa (e.g., adalimumab, infliximab, and golimumab), anti-RGMa (e.g., elezanumab), anti-TTR (e.g., NI-301 and PRX-004), anti-CTGF (e.g., pamrevlumab), anti- IL6R (e.g., satralizumab, tocilizumab, and sarilumab), anti-IL6 (e.g. siltuximab, clazakizumab, sirukumab, olokizumab, and gerilimzumab), anti-IL4R (e.g., dupilumab), anti-IL17A (e.g., ixekizumab and secukinumab), anti-IL5R (e.g. reslizumab), anti-IL-5 (e.g., benralizumab and mepolizumab), anti-IL13 (e.g. tralokinumab), anti-IL12/IL23 (e.g., ustekinumab), anti-CD 19 (e.g., inebilizumab), anti-IL31RA (e.g. nemolizumab), anti-ITGF7 mAb (e.g., etrolizumab), anti-SOST mAb (e.g., romosozumab), anti-IgE (e.g. omalizumab), anti-TSLP (e.g. nemolizumab), anti-pKal mAb (e.g., lanadelumab), anti-ITGA4 (e.g., natalizumab), anti- ITGA4B7 (e.g., vedolizumab), anti-BLyS (e.g., belimumab), anti-PD-1 (e.g., nivolumab and pembrolizumab), anti-RANKL (e.g., denosumab), anti-PCSK9 (e.g., alirocumab and evolocumab), anti-ANGPTL3 (e.g., evinacumab*), anti-OxPL (e.g., E06), anti-fD (e.g., lampalizumab), or anti-MMP9 (e.g., andecaliximab), optionally wherein the heavy chain (Fab and Fc region) and the light chain are separated by a self-cleaving furin (F)/F2A or furin (F)/T2A, IRES site, or flexible linker, for example, ensuring expression of equal amounts of the heavy and the light chain polypeptides.

In embodiments, the payload comprises a nucleic acid encoding a gene product linked to a disorder of the eye, or a fragment thereof. Exemplary gene products linked to a disorder of the eye include, for example, ADP-ribosylation factor-like 6 (ARL6); BBSome interacting protein 1 (BBIP1); BBSome protein 1 (BBS1); BBSome protein 2 (BBS2); BBSome protein 4 (BBS4); BBSome protein 5 (BBS5); BBSome protein 7 (BBS7); BBSome protein 9 (BBS9); BBSome protein 10 (BBS10); BBSome protein 12 (BBS12); centrosomal protein 290 kDa (CEP290); intraflagellar transport protein 172 (IFT172); intraflagellar transport protein 27 (IFT27); inositol polyphosphate-5-phosphatase E (INPP5E); inwardly-rectifying potassium channel subfamily J member 13 (KCNJ13); leucine zipper transcription factor like-1 (LZTFL1); McKusick-Kaufman syndrome protein (MKKS); Meckel syndrome type 1 protein (MKS1); nephronophthisis 3 protein (NPHP1); serologically-defined colon cancer antigen 8 (SDCCAG8); tripartite motif-containing protein 32 (TRIM32); tetratricopeptide repeat domain 8 (TTC8); Batten disease protein (CLN3); cytochrome P450 4V2 (CYP4V2); Rab escort protein 1 (CHM); PR (positive regulatory) domain-containing 13 protein (PRDM13); RPE-retinal G protein-coupled receptor (RGR); TEA domain family member 1 (TEAD1); arylhydrocarbon-interacting receptor protein-like 1 (AIPL1); cone-rod otx-like photoreceptor homeobox transcription factor (CRX); guanylate cyclase activating protein 1A (GUCA1A); retinal-specific guanylate cyclase (GUCY2D); phosphatidylinositol transfer membrane-associated family member 3 (PITPNM3); prominin 1 (PROM1); peripherin (PRPH); peripherin 2 (PRPH2); regulating synaptic membrane exocytosis protein 1 (RIMS1); semaphorin 4A (SEMA4A); human homolog of C. elegans unc119 protein (UNC119); ATP-binding cassette transporter-retinal (ABCA4); ADAM metallopeptidase domain 9 (ADAM9); activating transcription factor 6 (ATF6); chromosome 21 open reading frame 2 (C21orf2); chromosome 8 open reading frame 37 (C8orf37); calcium channel; voltage-dependent; alpha 2/delta subunit 4 (CACNA2D4); cadherin-related family member 1 (protocadherin 21) (CDHR1); ceramide kinase-like protein (CERKL); cone photoreceptor cGMP-gated cation channel alpha subunit (CNGA3); cone cyclic nucleotide-gated cation channel beta 3 subunit (CNGB3); cyclin M4 (CNNM4); guanine nucleotide binding protein (G protein); alpha transducing activity polypeptide 2 (GNAT2); potassium channel subfamily V member 2 (KCNV2); Phosphodiesterase 6C (PDE6C); Phosphodiesterase 6H (PDE6H); proteome of centriole 1 centriolar protein B (POCIB); RAB28 member of RAS oncogene family (RAB28); retina and anterior neural fold homeobox 2 transcription factor (RAX2); 11-cis retinol dehydrogenase 5 (RDH5); RP GTPase regulator-interacting protein 1 (RPGRIP1); tubulin tyrosine ligase-like family member 5 (TTLL5); L-type voltage-gated calcium channel alpha-1 subunit (CACNA1F); retinitis pigmentosa GTPase regulator (RPGR); rod transducin alpha subunit (GNAT1); rod cGMP phosphodiesterase beta subunit (PDE6B); rhodopsin (RHO); calcium binding protein 4 (CABP4); G protein-coupled receptor 179 (GPR179); rhodopsin kinase (GRK1); metabotropic glutamate receptor 6 (GRM6); leucine-rich repeat immunoglobulin-like transmembrane domains protein 3 (LRIT3); arrestin (s-antigen) (SAG); solute carrier family 24 (SLC24A1); transient receptor potential cation channel, subfamily M, member 1 (TRPM1); nyctalopin (NYX); green cone opsin (OPN1LW); red cone opsin (OPN1MW); blue cone opsin (OPNlSW); frataxin (FXN); inosine monophosphate dehydrogenase 1 (IMPDH1); orthodenticle homeobox 2 protein (OTX2); crumbs homolog 1 (CRB1); death domain containing protein 1 (DTHD1); growth differentiation factor 6 (GDF6); intraflagellar transport 140 Chlamydomonas homolog protein (IFT140); IQ motif containing B protein (IQCB1); lebercilin (LCA5); lecithin retinol acyltransferase (LRAT); nicotinamide nucleotide adenylyltransferase 1 (NMNAT1); RD3 protein (RD3); retinol dehydrogenase 12 (RDH12); retinal pigment epithelium-specific 65 kD protein (RPE65); spermatogenesis associated protein 7 (SPATA7); tubby-like protein 1 (TULP1); mitochondrial genes (KSS, LHON, MT-ATP6, MT-TH, MT-TL1, MT-TP, MT-TS2, mitochondrially encoded NADH dehydrogenases [MT-ND]); bestrophin 1 (BEST1); C1q and tumor necrosis-related protein 5 collagen (C1QTNF5); EGF-containing fibrillin-like extracellular matrix protein 1 (EFEMP1); elongation of very long fatty acids protein (ELOVL4); retinal fascin homolog 2, actin bundling protein (FSCN2); guanylate cyclase activating protein 1B (GUCAB); hemicentin 1 (HMCN1); interphotoreceptor matrix proteoglycan 1 (IMPG1); retinitis pigmentosa 1-like protein 1 (RP1L1); tissue inhibitor of metalloproteinases-3 (TIMP3); complement factor H (CFH); complement factor D (CFD); complement component 2 (C2); complement component 3(C3); complement factor B (CFB); DNA-damage regulated autophagy modulator 2 (DRAM2); chondroitin sulfate proteoglycan 2 (VCAN); mitofusin 2 (MFN2); nuclear receptor subfamily 2 group F member 1 (NR2F1); optic atrophy 1 (OPA1); transmembrane protein 126A (TMEM126A); inner mitochondrial membrane translocase 8 homolog A (TIMM8A); carbonic anhydrase IV (CA4); hexokinase 1 (HK1); kelch-like 7 protein (KLHL7); nuclear receptor subfamily 2 group E3 (NR2E3); neural retina lucine zipper (NRL); olfactory receptor family 2 subfamily W member 3 (OR2W3); pre-mRNA processing factor 3 (PRPF3); pre-mRNA processing factor 4 (PRPF4); pre-mRNA processing factor 6 (PRPF6); pre-mRNA processing factor 8 (PRPF8); pre-mRNA processing factor 31 (PRPF31); retinal outer segment membrane protein 1 (ROM1); retinitis pigmentosa protein 1 (RP1); PIM-kinase associated protein 1 (RP9); small nuclear ribonucleoprotein 200 kDa (SNRNP200); secreted phosphoprotein 2 (SPP2); topoisomerase I binding arginine/serine rich protein (TOPORS); ADP-ribosylation factor-like 2 binding protein (ARL2BP); chromosome 2 open reading frame 71 (C2orf71); clarin-1 (CLRN1); rod cGMP-gated channel alpha subunit (CNGA1); rod cGMP-gated channel beta subunit (CNGB1); cytochrome P450 4V2 (CYP4V2); dehydrodolichyl diphosphate synthetase (DHDDS); DEAH box polypeptide 38 (DHX38); ER membrane protein complex subunit 1 (EMC1); eyes shut/spacemaker homolog (EYS); family with sequence similarity 161 member A (FAM161A); G protein-coupled receptor 125 (GPR125); heparan-alpha-glucosaminide N-acetyltransferase (HGSNAT); NAD(+)-specific isocitrate dehydrogenase 3 beta (IDH3B); interphotoreceptor matrix proteoglycan 2 (IMPG2); KIAA1549 protein (KIAA1549); kizuna centrosomal protein (KIZ); male germ-cell associated kinase (MAK); c-mer protooncogene receptor tyrosine kinase (MERTK); mevalonate kinase (MVK); NIMA (never in mitosis gene A)-related kinase 2 (NEK2); neuronal differentiation protein 1 (NEUROD1); cGMP phosphodiesterase alpha subunit (PDE6A); phosphodiesterase 6G cGMP-specific rod gamma (PDE6G); progressive rod-cone degeneration protein (PRCD); retinol binding protein 3 (RBP3); retinaldehyde-binding protein 1 (RLBP1); solute carrier family 7 member 14 (SLC7A14); usherin (USH2A); zinc finger protein 408 (ZNF408); zinc finger protein 513 (ZNF513); oral-facial-digital syndrome 1 protein (OFD1); retinitis pigmentosa 2 (RP2); retinoschisin (RS1); abhydrolase domain containing protein 12 (ABHD12); cadherin-like gene 23 (CDH23); centrosomal protein 250 kDa (CEP250); calcium and integrin binding family member 2 (CIB2); whirlin (DFNB31); monogenic audiogenic seizure susceptibility 1 homolog (GPR98); histidyl-tRNA synthetase (HARS); myosin VIIA (MYO7A); protocadherin 15 (PCDH15); harmonin (USH1C); human homolog of mouse scaffold protein containing ankyrin repeats and SAM domain (USH1G); dystrophin (DMD); norrin (NDP); phosphoglycerate kinase (PGK1); calpain 5 (CAPN5); frizzled-4 Wnt receptor homolog (FZD4); integral membrane protein 2B (ITM2B); low density lipoprotein receptor-related protein 5 (LRP5); micro RNA 204 (MIR204); retinoblastoma protein 1 (RB1); tetraspanin 12 (TSPAN12); chromosome 12 open reading frame 65 (C12orf65); cadherin 3 (CDH3); membrane-type frizzled-related protein (MFRP); ornithine aminotransferase (OAT); phospholipase A2 group V (PLA2G5); retinol-binding protein 4 (RBP4); regulator of G-protein signaling 9 (RGS9); regulator of G-protein signaling 9-binding protein (RGS9BP); ARMS2; excision repair cross-complementing rodent repair deficiency complementation group 6 protein (ERCC6); fibulin 5 (FBLN5); HtrA serine peptidase 1 (HTRA1); toll-like receptor 3 (TLR3); and toll-like receptor 4 (TLR4), opsin; rhodopsin; channel rhodopsin; halo rhodopsin, and the like.

In some embodiments, the virus particle comprises a heterologous transgene encoding a genome editing system. Examples include a CRISPR genome editing system (e.g., one or more components of a CRISPR genome editing system such as, for example, a guide RNA molecule and/or a RNA-guided nuclease such as a Cas enzyme such as Cas9, Cpf1 and the like), a zinc finger nuclease genome editing system, a TALEN genome editing system or a meganuclease genome editing system. In embodiments, the genome editing system targets a mammalian, e.g., human, genomic target sequence. In embodiments, the virus particle includes a heterologous transgene encoding a targetable transcription regulator. Examples include a CRISPR-based trascription regulator (for example, one or more components of a CRISPR-based transcription regulator, for example, a guide RNA molecule and/or a enzymatically-inactive RNA-guided nuclease/transcription factor (“TF”) fusion protein such as a dCas9-TF fusion, dCpf1-TF fusion and the like), a zinc finger transcription factor fusion protein, a TALEN transcription regulator or a meganuclease transcription regulator.

In some embodiments, components of a therapeutic molecule or system are delivered by more than one unique virus particle (e.g., a population that includes more than one unique virus particles). In other embodiments, the therapeutic molecule or components of a therapeutic molecule or system are delivered by a single unique virus particle (e.g., a population that includes a single unique virus particle).

The transgene may also encode any biologically active product or other product, e.g., a product desirable for study. Suitable transgenes may be readily selected by persons of skill in the art, such as those, but not limited to, those described herein.

Other examples of proteins encoded for by the transgene include, but are not limited to, colony stimulating factors (CSF); blood factors, such as P-globin, hemoglobin, tissue plasminogen activator, and coagulation factors; interleukins; soluble receptors, such as soluble TNF-α. receptors, soluble VEGF receptors, soluble interleukin receptors (e.g., soluble IL-1 receptors and soluble type II IL-1 receptors), or ligand-binding fragments of a soluble receptor; growth factors, such as keratinocyte growth factor (KGF), stem cell factor (SCF), or fibroblast growth factor (FGF, such as basic FGF and acidic FGF); enzymes; chemokines, enzyme activators, such as tissue plasminogen activator; angiogenic agents, such as vascular endothelial growth factors, glioma-derived growth factor, angiogenin, or angiogenin-2; anti-angiogenic agents, such as a soluble VEGF receptor; a protein vaccine; neuroactive peptides, such as nerve growth factor (NGF) or oxytocin; thrombolytic agents; tissue factors; macrophage activating factors; tissue inhibitors of metalloproteinases; or IL-1 receptor antagonists.

Accordingly, provided herein is a virus particle comprising a capsid polypeptide comprising (a) a VP1, VP2 or VP3 sequence of SEQ ID NO: 2, (b) a VP1, VP2 or VP3 sequence comprising the mutation set of VAR-1 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-1 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 2. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 3, (b) a VP1, VP2 or VP3 sequence comprising the mutation set of VAR-2 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-2 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 3. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

Accordingly, provided herein is a virus particle comprising a capsid polypeptide comprising (a) a VP1, VP2 or VP3 sequence of SEQ ID NO: 4, (b) a VP1, VP2 or VP3 sequence comprising the mutation set of VAR-3 and having greater than 80% (for example, greater than 90% greater than 91%, greater than 92%, greater than 93%, greater than 94%, greater than 95%, greater than 96%, greater than 97%, greater than 98%, greater than 99%) identity to SEQ ID NO: 1, or (c) a VP1, VP2 or VP3 sequence comprising the mutation set of VAR-3 and having at least 1, 2, 3, 4, 5, 6, 7, 8, 9 or 10 additional mutations, but fewer than 40, 39, 38, 37, 36, 35, 34, 33, 32 or 31 additional mutations relative to SEQ ID NO: 1. In embodiments, the capsid polypeptide comprises VP1, VP2 and VP3 sequences of SEQ ID NO: 4. In embodiments, the virus particle comprises a nucleic acid molecule comprising a heterologous transgene, for example a heterologous transgene encoding a product directed to an ocular disorder. In embodiments, the heterologous transgene encodes an anti-VEGF antibody or antibody fragment, an anti-VEGF RNA inhibitory molecule, a RPE65 (e.g., human RPE65) protein, a ABCA4 (e.g., human ABCA4) protein or fragment thereof, a RLBP1 (e.g., human RLBP1) protein or fragment thereof, a PDE6B (e.g., human PDE6B) protein or fragment thereof, a RPGR (e.g., human RPGR) protein or fragment thereof or a ACHM3A or ACHM3B (e.g., human ACHM3A or human ACHM3B) protein or fragment thereof. In embodiments, the nucleic acid molecule of the virus particle further comprises one or more regulatory elements, e.g., comprises a promoter, e.g., a promoter operably linked to the heterologous transgene and which regulates expression from the heterologous transgene in a tissue of interest. In embodiments, the nucleic acid molecule of the virus particle further comprises one or more of (a) a dependoparvovirus ITR, (b) an intron, (c) an enhancer or repressor sequence, (d) a stuffer sequence, and (e) a polyA sequence.

The disclosure is further directed, in part, to a method of delivering a payload to a subject, e.g., an animal or human subject. In some embodiments, a method of delivering a payload to a subject comprises administering to the subject a dependoparvovirus particle comprising a variant polypeptide (e.g., described herein) comprising the payload, e.g., in a quantity and for a time sufficient to deliver the payload. In some embodiments, the dependoparvovirus particle is a dependoparvovirus particle described herein and comprises a payload described herein. In some embodiments, the particle delivers the payload to the eye. In some embodiments, the delivery to the eye is increased as compared to a particle without the variant capsid polypeptide or as compared to a wild-type capsid polypeptide.

### Methods of Treatment

The disclosure is directed, in part, to a method of treating a disease or condition in a subject, e.g., an animal or human subject. As used herein, the term “treating a disease or condition” refers to treating a manifest disease or condition, for example, where the subject is already suffering from one or more symptoms of the disease or condition, or refers to treating a pre-manifest disease or condition, for example, where the subject is identified as having a disease or condition but is not yet exhibiting one or more symptoms of the disease or condition. Pre-manifest conditions may be identified by, for example, genetic testing. In some embodiments, a method of treating a disease or condition in a subject comprises administering to the subject a dependoparvovirus particle comprising a variant polypeptide described herein, e.g., comprising a payload described herein. In some embodiments, the dependoparvovirus particle, which comprises a variant polypeptide, comprising a payload described herein is administered in an amount and/or time effective to treat the disease or condition. In some embodiments, a method of treating a CNS and/or ocular disease or condition in a subject comprises administering to the subject a dependoparvovirus particle comprising a variant polypeptide described herein, e.g., comprising a payload described herein. In some embodiments, the dependoparvovirus particle, which comprises a variant polypeptide, comprising a payload described herein is administered in an amount and/or time effective to treat the CNS and/or ocular disease or condition. In some embodiments, the CNS and/or ocular disease or condition is neuronal ceroid lipofucsinosis (NCL). In some embodiments, the dependoparvovirus particle, which comprises a variant polypeptide, comprising a payload described herein is administered in an amount and/or time effective to treat the CNS and/or ocular disease or condition, optionally wherein the disease or condition is a neuronal ceroid lipofucsinosis (NCL). In some embodiments, a method of treating neuronal ceroid lipofucsinosis (NCL) in a subject comprises administering to the subject a dependoparvovirus particle comprising a variant polypeptide described herein, e.g., comprising a payload described herein. In some embodiments, a method of treating a CNS disease or condition in a subject comprises administering to the subject a dependoparvovirus particle comprising a variant polypeptide described herein, e.g., comprising a payload described herein. In some embodiments, the dependoparvovirus particle, which comprises a variant polypeptide, comprising a payload described herein is administered in an amount and/or time effective to treat the CNS disease or condition. In some embodiments, a method of treating an ocular disease or condition in a subject comprises administering to the subject a dependoparvovirus particle comprising a variant polypeptide described herein, e.g., comprising a payload described herein. In some embodiments, the dependoparvovirus particle, which comprises a variant polypeptide, comprising a payload described herein is administered in an amount and/or time effective to treat the ocular disease or condition.

In some embodiments, the payload is a therapeutic product. In some embodiments, the payload is a nucleic acid, e.g., encoding an exogenous polypeptide.

The dependoparvovirus particles comprising a variant polypeptide described herein or produced by the methods described herein can be used to express one or more therapeutic proteins to treat various diseases or disorders. In some embodiments, the disease or disorder is a cancer, e.g., a cancer such as carcinoma, sarcoma, leukemia, lymphoma; or an autoimmune disease, e.g., multiple sclerosis. Non-limiting examples of carcinomas include esophageal carcinoma; bronchogenic carcinoma; colon carcinoma; colorectal carcinoma; gastric carcinoma; hepatocellular carcinoma; basal cell carcinoma, squamous cell carcinoma (various tissues); bladder carcinoma, including transitional cell carcinoma; lung carcinoma, including small cell carcinoma and non-small cell carcinoma of the lung; adrenocortical carcinoma; sweat gland carcinoma; sebaceous gland carcinoma; thyroid carcinoma; pancreatic carcinoma; breast carcinoma; ovarian carcinoma; prostate carcinoma; adenocarcinoma; papillary carcinoma; papillary adenocarcinoma; cystadenocarcinoma; medullary carcinoma; renal cell carcinoma; uterine carcinoma; testicular carcinoma; osteogenic carcinoma; ductal carcinoma in situ or bile duct carcinoma; choriocarcinoma; seminoma; embryonal carcinoma; Wilm's tumor; cervical carcinoma; epithelieal carcinoma; and nasopharyngeal carcinoma. Non-limiting examples of sarcomas include fibrosarcoma, myxosarcoma, liposarcoma, angiosarcoma, endotheliosarcoma, lymphangiosarcoma, chondrosarcoma, chordoma, osteogenic sarcoma, osteosarcoma, lymphangioendotheliosarcoma, synovioma, mesothelioma, Ewing's sarcoma, leiomyosarcoma, rhabdomyosarcoma, and other soft tissue sarcomas. Non-limiting examples of solid tumors include ependymoma, pinealoma, hemangioblastoma, acoustic neuroma, oligodendroglioma, glioma, astrocytoma, medulloblastoma, craniopharyngioma, menangioma, melanoma, neuroblastoma, and retinoblastoma. Non-limiting examples of leukemias include chronic myeloproliferative syndromes; T-cell CLL prolymphocytic leukemia, acute myelogenous leukemias; chronic lymphocytic leukemias, including B-cell CLL, hairy cell leukemia; and acute lymphoblastic leukemias. Examples of lymphomas include, but are not limited to, B-cell lymphomas, such as Burkitt's lymphoma; and Hodgkin's lymphoma.

In some embodiments, the disease or disorder is a genetic disorder. In some embodiments, the genetic disorder is sickle cell anemia, Glycogen storage diseases (GSD, e.g., GSD types I, II, III, IV, V, VI, VII, VIII, IX, X, XI, XII, XIII, and XIV), cystic fibrosis, lysosomal acid lipase (LAL) deficiency 1, Tay-Sachs disease, Phenylketonuria, Mucopolysaccharidoses, Galactosemia, muscular dystrophy (e.g., Duchenne muscular dystrophy), hemophilia such as hemophilia A (classic hemophilia) or hemophilia B (Christmas Disease), Wilson's disease, Fabry Disease, Gaucher Disease hereditary angioedema (HAE), and alpha 1 antitrypsin deficiency. Examples of other diseases or disorders are provided above in the “Methods of delivering a payload” section.

The dependoparvovirus particles comprising a variant polypeptide described herein or produced by the methods described herein can be used to express one or more therapeutic proteins to treat various diseases or disorders. In some embodiments, the disease or disorder is a disease or disorder of the eye, for example, retinitis pigmentosa; macular degeneration (e.g.; wet age-related macular degeneration), optic neuritis; Leber's congenital amaurosis; Leber's hereditary optic neuropathy; achromatopsia; X-linked retinoschisis; optic neuritis; choroideremia; optic atrophy; retinal cone dystrophy; retinopathy; retinoblastoma; glaucoma; Bardet-Biedl syndrome; Usher syndrome; aniridia; Friedreich's ataxia; vitelliform macular dystrophy; retinoblastoma; Stargardt disease; Charcot-Marie-Tooth disease; Fuch's dystrophy; propionic acidemia; or color blindness; corneal dystrophy; keratoconus; night blindness; dry eye; Bardet-Biedl syndrome; Batten's Disease; Bietti's Crystalline Dystrophy; chorioretinal atrophy; chorioretinal degeneration; cone or cone-rod dystrophies (autosomal dominant, autosomal recessive, and X-linked), congenital stationary night blindness (autosomal dominant, autosomal recessive, and X-linked); disorders of color vision, including achromatopsia (including ACHM2, ACHM3, ACHM4, and ACHM5), protanopia, deuteranopia, and tritanopia; Friedreich's ataxia; Leber's congenital amaurosis (autosomal dominant and autosomal recessive), including, but not limited to, LCA1, LCA2, LCA3, LCA4, LCA6, LCA7, LCA8, LCA12, and LCA15; Leber's Hereditary Optic Neuropathy; macular dystrophy (autosomal dominant and autosomal recessive), including, but not limited to, acute macular degeneration, Best vitelliform macular dystrophy, pattern dystrophy, North Carolina Macular Dystrophy, inherited drusen, Sorsby's fundus dystrophy, malattia levantanese, and genetically-determined retinopathy of prematurity; ocular-retinal developmental disease; ocular albinism; optic atrophies (autosomal dominant, autosomal recessive, and X-linked); retinitis pigmentosa (autosomal dominant, autosomal recessive, X-linked, and mitochondrially-inherited traits), examples of which include RP1, RP2, RP3, RP1O, RP20, RP38, RP40, and RP43; X-linked retinoschisis; Stargardt disease; and Usher syndrome, including, but not limited to, USHIB, USHIC, USHID, USHIF, USHIG, USH2A, USH2C, USH2D, AND USH3. Examples of complex genetic diseases include, but are not limited to, glaucoma (open angle, angle-closure, low-tension, normal-tension, congenital, neovascular, pigmentary, pseudoexfoliation); age-related and other forms of macular degeneration, both exudative and non-exudative forms (autosomal dominant and autosomal recessive), such as acute macular degeneration, vitelliform macular degeneration; retinopathy of prematurity; and Vogt Koyanagi-Harada (VKH) syndrome. Examples of acquired diseases include, but are not limited to, acute macular neuroretinopathy; anterior ischemic optic neuropathy and posterior ischemic optic neuropathy; Behcet's disease; branch retinal vein occlusion; choroidal neovascularization; diabetic retinopathy, including proliferative diabetic retinopathy and associated complications; diabetic uveitis; edema, such as macular edema, cystoid macular edema and diabetic macular edema; epiretinal membrane disorders; macular telangiectasia; multifocal choroiditis; non-retinopathy diabetic retinal dysfunction; ocular tumors; optic atrophies; retinal detachment; retinal disorders, such as central retinal vein occlusion, proliferative vitreoretinopathy (PVR), retinal arterial and venous occlusive disease, vascular occlusion, uveitic retinal disease; uveal effusion; retinal infective and infiltrative disease; optic nerve diseases such as acquired optic atrophy. Examples of traumatic injuries include, but are not limited to, histoplasmosis; optic nerve trauma; ocular trauma which affects a posterior ocular site or location; retinal trauma; viral infection of the eye; viral infection of the optic nerve; a posterior ocular condition caused by or influenced by an ocular laser treatment; posterior ocular conditions caused by or influenced by a photodynamic therapy; photocoagulation, radiation retinopathy; and sympathetic ophthalmia.

In some embodiments, administration of a dependoparvovirus particle comprising a variant polypeptide and comprising a payload (e.g., a transgene) to a subject induces expression of the payload (e.g., transgene) in a subject. In some embodiments, the expression is induced in the eye. In some embodiments, the production is increased in the eye as compared to a similar particle with the wild-type capsid protein. The amount of a payload, e.g., transgene, e.g., heterologous protein, e.g., therapeutic polypeptide, expressed in a subject (e.g., the serum of the subject) can vary. For example, in some embodiments the payload, e.g., protein or RNA product of a transgene, can be expressed in the serum of the subject in the amount of less than about 5 g/ml. For example, in some embodiments the payload, e.g., protein or RNA product of a transgene, can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 g/ml, at least about 700 μg/ml, at least about 800 μg/ml, at least about 900 μg/ml, or at least about 1000 μg/ml. In some embodiments, the payload, e.g., protein or RNA product of a transgene, is expressed in the serum of the subject in the amount of about 9 μg/ml, about 10 g/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values.

In some embodiments, a dependoparvovirus particle comprising a variant polypeptide and comprising a payload (e.g., a transgene) is administered to a subject via an injection. In some embodiments, the injection is a systemic injection, for example, intravenous, intraarterial, intramuscular, or subcutaneous injection. In embodiments, the injection is by intravenous injection. In some embodiments, the injection is an injection to the eye. In some embodiments, the injection is an intravitreal injection, intraorbital injection, retro-orbital injection, suprachoroidal injection, subretinal injection, subconjunctivital injection, or intracameral injection. In some embodiments, the injection is an intravitreal injection. In some embodiments, the injection is an intraorbital injection. In some embodiments, the injection is a retro-orbital injection. In some embodiments, the injection is a suprachoroidal injection. In some embodiments, the injection is a subretinal injection. In some embodiments, the injection is a subconjunctivital injection. In some embodiments, the injection is an intracameral injection. In some embodiments, the injection is an intravenous injection. One example of an intravenous injection which is conttemplated in the methods of the present invention is by interventional radiography-guided intravenous administration. In embodiments, such administration is via guided catheter inserted into an artery providing blood flow to the eye (for example via the carotid artery, or to any of the lesser arteries stemming therefrom (e.g., via the ophthalmic artery)). Without being bound by theory, such “local” intravenous administration reduces the amount of virus particle introduced into the body by targeting delivery to the site of interest.

Sequences disclosed herein may be described in terms of percent identity. A person of skill will understand that such characteristics involve alignment of two or more sequences.

Alignments may be performed using any of a variety of publicly or commercially available Multiple Sequence Alignment Programs, such as “Clustal W”, accessible via the Internet. As another example, nucleic acid sequences may be compared using FASTA, a program in GCG Version 6.1. FASTA provides alignments and percent sequence identity of the regions of the best overlap between the query and search sequences. For instance, percent identity between nucleic acid sequences may be determined using FASTA with its default parameters as provided in GCG Version 6.1, herein incorporated by reference. Similar programs are available for amino acid sequences, e.g., the “Clustal X” program. Additional sequence alignment tools that may be used are provided by (protein sequence alignment; (http://www.ebi.ac.uk/Tools/psa/emboss_needle/)) and (nucleic acid alignment; http://www.ebi.ac.uk/Tools/psa/emboss_needle/nucleotide.html)). Generally, any of these programs may be used at default settings, although one of skill in the art can alter these settings as needed. Alternatively, one of skill in the art can utilize another algorithm or computer program which provides at least the level of identity or alignment as that provided by the referenced algorithms and programs. Sequences disclosed herein may further be described in terms of edit distance. The minimum number of sequence edits (i.e., additions, substitutions, or deletions of a single base or nucleotide) which change one sequence into another sequence is the edit distance between the two sequences. In some embodiments, the distance between two sequences is calculated as the Levenshtein distance.

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Aug. 21, 2020. When one gene or protein references a plurality of sequence accession numbers, all of the sequence variants are encompassed.

The invention is further illustrated by the following examples. The examples are provided for illustrative purposes only and are not to be construed as limiting the scope or content of the invention in any way.

### EXAMPLES

### Example 1

### Library Creation

A library of 2.5E5 capsid variants of wild—type AAV2 were designed and cloned into plasmids to create a library of plasmids encoding the capsid variants. A library of AAV variant genomes encoding each variant's capsid and a unique capsid variant barcode identifier was cloned into three ITR plasmid backbones as described previously (Ogden et al. 2019). Each plasmid backbone contained a unique genomic identifier enabling analysis of biodistribution and transduction efficiencies via different routes of administration. The libraries were produced via transient triple transfection of adherent HEK293T followed by iodixanol gradient purification.

### In Vitro Evaluation of Library

Data was prepared as described below. To measure each variant's packaging efficiency (or “production”), barcodes from vector genomes in the plasmid and produced AAV library were prepared for Illumina sequencing using two rounds of PCR. Production efficiency, normalized for presence in the input plasmid library, for each variant is expressed by comparing barcode sequencing levels for each variant in the produced vector pool to the barcode sequence levels for each variant in the input plasmid library used to create the vector pool. The measurements of variant frequency in the vector library also enable downstream normalization of biodistribution and transduction measurements by variant frequency in the input vector library. Production efficiency is reported in Table 1, and each reported value is reported as the log 2 production relative to the production of wild-type AAV2.

### In Vivo Evaluation of Library in Non-Human Primate

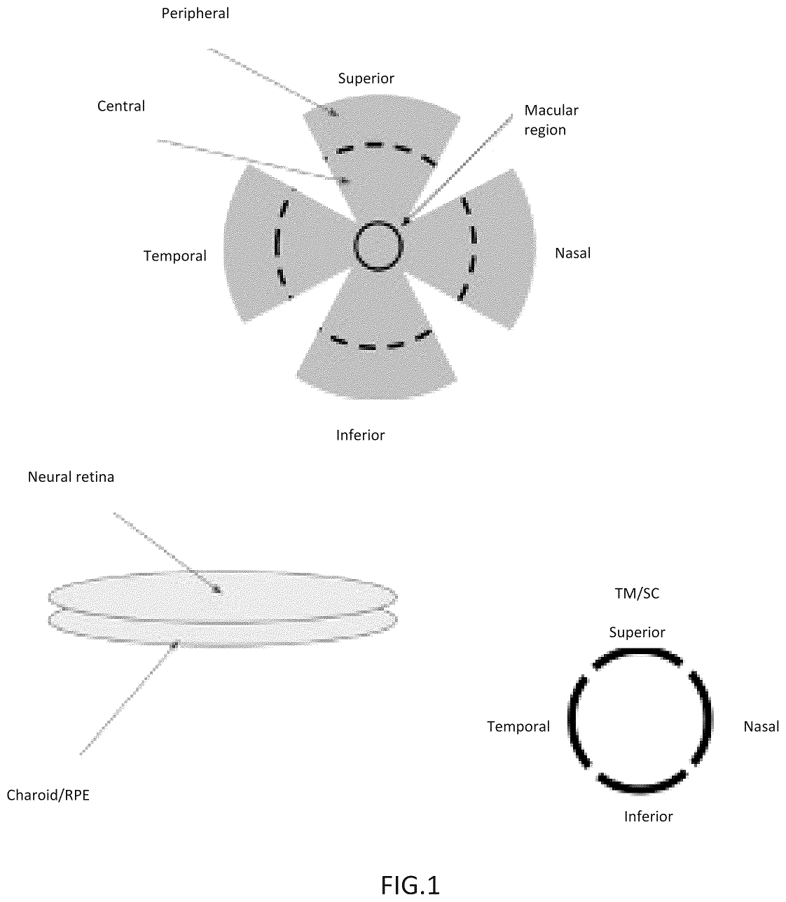

All NHP experiments were conducted in accordance with institutional policies and NIH guidelines. One young adult male and one young adult female cynomolgus macaque (Macaca fascicularis) weighing 2.4-2.9 kg seronegative for anti-AAV2 neutralizing antibodies (serum NAb titers <1:20 based on in vitro NAb assay) were selected for the study. Prior to test article administrations samples of blood, aqueous humor (50 μL) and vitreous humor (up to 50 μL) were collected. The animals were anesthetized with ketamine and dexmedetomidine and received intravitreal (IVT; 4.8E11 vg/eye in 50 μL), intracameral (IC; 8.5E11 vg/eye in 50 μL) and intravenous (IV; 1.8-2.5E13 vg/kg) injections of the vector libraries. During the in-life period the animals were monitored for signs of ocular inflammation via indirect ophthalmoscopy and slit-lamp biomicroscopy and treated with weekly IM injections of steroids (methylprednisolone, 40-80 mg) and topical steroids (Durezol), and atropine as needed according to the animal facility's SOPs and recommendations from the veterinarian. Serum samples were collected at 1 h, 4 h and 24 h, and weekly after the injections. The animals were sacrificed 4 weeks after the injections and tissues were collected for biodistribution and transduction analyses. Retinas and trabecular meshwork were dissected as shown in FIG. 1. A list of other tissue samples collected is shown in Table 3. All samples were collected into RNAlater® (Sigma-Aldrich) and incubated overnight at RT, after which the RNAlater® was drained and samples were frozen at −80° C. In addition, samples of aqueous humor, vitreous humor, serum, and cerebrospinal fluid were collected at necropsy and stored at −80° C.

TABLE 3
List of tissues collected.
Tissue

Adrenal gland
brain (cortical slices) coronal axis
dorsal root ganglion (cervical)
dorsal root ganglion (thoracic)
dorsal root ganglion (lumbar)
gonad (testes and ovaries)
heart, basal (left atrium)
heart, apex
heart, right ventricle
kidney
liver
Lung (superior lobe)
lymph nodes, cervical
skeletal muscle, bicep brachii
skeletal muscle, diaphragm
skeletal muscle, quadriceps
spinal cord (cervical)
spinal cord (thoracic)
spinal cord (lumbar)
spleen

For biodistribution and transduction analyses, total DNA and RNA was extracted from tissue samples with Trizol/chloroform and isopropanol precipitation. RNA samples were treated with TURBO DNase (Invitrogen). Reverse transcription was done with Protoscript II Reverse Transcriptase (NEB) with primers that were specific to the vector transgene and included unique molecular identifiers (UMIs). Control reactions lacking the reverse transcriptase enzyme (-RT control) were also prepared. Quantification of biodistribution and transduction was done with Luna Universal Probe qPCR Master Mix (NEB) using primers and probes specific to the transgene construct. Finally, samples were prepared for next-generation sequencing by amplifying the transgene barcode regions with primers compatible with Illumina NGS platform and sequenced with NextSeq 550 (Illumina).

After sequencing, the barcode tags were extracted from reads with the expected amplicon structure, and the abundance (number of reads or number of UMIs) of each barcode was recorded. Analyses were restricted to the set of barcodes that were present in the input plasmid sample and that did not contain errors in the variant sequence, as measured by a separate sequencing assay that targeted the variant regions of the input plasmid sample.

To aggregate packaging replicates, the read counts from replicate virus production samples were summed. To aggregate transduction samples, the UMI counts from samples from the same tissue were summed.

Virus packaging, biodistribution and transduction of tissue were calculated using a Bayesian model with aggregated production, biodistribution and/or transduction samples as the input. Briefly, probabilistic programming and stochastic variational inference were used to model the measurement process and sources of decoupling (e.g., cross-packaging, template switching, and errors in DNA synthesis) between the actual test virus particles and their designed sequences, and to calculate virus production, biodistribution and transduction (in various tissue samples), and error rates. The output was the log 2-transformed mean of the calculated distribution relative to the wild-type (WT) AAV2. Thus, positive values indicate better performance than WT for the measured property, and negative values indicate worse-than-WT performance. Transduction is reported in Table 1.

### Example 2

The virus particles comprising the variant capsids provided in Table 2 (sequences) are produced individually via transient triple transfection of adherent HEK293T followed by iodixanol gradient purification. Each variant capsid is produced with a genome encoding a unique barcode and a fluorescent reporter gene under the control of a ubiquitous promoter. Production efficiency is assessed as described above. Equivalent amounts (vg) of each virus particle are pooled and injected into Non-human primates (e.g., cynomolgus macaque or African green monkey) at doses used in Example 1. Virus properties, including biodistribution and tissue transduction are assessed, for example, as described in Example 1.

The virus particles comprising a selection of capsids (approximately 100 unique variants and wild-type comparators), including those provided in Table 2 (sequences), were produced individually via transient triple transfection of adherent HEK293T followed by iodixanol gradient purification. Representation of individual variants within the final pooled test article were balanced to be within 10-fold range where possible. Each variant capsid was produced with a genome encoding a unique barcode and a fluorescent reporter gene under the control of a ubiquitous promoter (cbh). In all, each variant was produced with separate genomes comprising 8 unique barcodes, providing a measure of biological replicates within the study. All NHP experiments were conducted in accordance with institutional policies and NIH guidelines. Two young adult male cynomolgus macaques (Macaca fascicularis) weighing 2.8-3 kg, one seronegative (serum NAb titers <1:20 based on in vitro NAb assay) and one seropositive (1:128) for anti-AAV2 neutralizing antibodies were selected for the study. Prior to test article administrations samples of blood, aqueous humor (50 μL) and vitreous humor (up to 50 μL) were collected. The animals were anesthetized with ketamine and dexmedetomidine and received intravenous injections (IV; 2E12vg/kg), intravitreal (IVT; 2.63E11 vg/eye in 50 μL) and intracameral (IC; 1.11E11 vg/eye in 50 μL) injections of vector libraries. The variants described herein were included in both the IV and the IVT libraries with separate barcodes so that each variant could be tracked by each route of administration. During the in-life period the animals were monitored for signs of ocular inflammation via indirect ophthalmoscopy and slit-lamp biomicroscopy and treated with weekly IM injections of steroids (methylprednisolone, 80 mg) and topical steroids (Durezol), and atropine as needed according to the animal facility's SOPs and recommendations from the veterinarian. The animals were sacrificed 4 weeks after the injections and tissues were collected for biodistribution and transduction analyses. Ocular and peripheral tissues, including liver, were weighed and flash-frozen on dry ice following dissection. Tissues were processed, and biodistribution/transduction assessed as described in Example 1. The results are shown in Tables 4-6, and values were derived from at least 4 tissue pieces of the indicated organ from each of the two test animals (at least 8 samples total).

The data from this medium throughput experiment confirm the findings from the library experiment described in Example 1, and demonstrate that virus particles described herein, such as those comprising the capsid polypeptides of VAR-1, VAR-2 and VAR-3 exhibit enhanced ocular transduction relative to virus particles comprising wild-type AAV2 capsid polypeptides by intravenous delivery. These increases are most pronounced in the choroid and neural retina layers of the retina both including and excluding the macula, but transduction of the trabecular meshwork tissue is also enhanced relative to wild-type AAV2. Interestingly, these variants further exhibit substantially decreased liver transduction and biodistribution relative to AAV5 and AAV2 delivered intravenously, indicating that the virus particles comprising these capsid polypeptides specifically transduce cells of the ocular tissues. Interestingly, when directly injected into the eye via intravitreal injection, virus particles comprising capsid polypeptides of these variants transduce ocular tissues within about 2.5-fold of wild-type AAV2 injected via intravitreal administration, indicating a preferential enhancement in the ability to transduce ocular tissues via the bloodstream rather than the intravitreal space. Without being bound by theory, this suggests these variants may achieve transduction via a mechanism which may involve the ability to cross the blood brain barrier. These results indicate that capsid polypeptides and virus particles comprising these capsid polypeptides thus have enhanced utility as gene therapy vectors for therapies directed to occular disorders or where selective and enhanced transduction to ocular regions, including retina, macular and/or trabecular meshwork tissue is beneficial.

### Example 3

In order to further investigate the ability of virus particles comprising the capsid polypeptides described herein to cross the blood brain barrier, two brain tissue samples were isolated from the one non-human primate described in Example 2 which was seronegative for anti-wild-type AAV2 neutralizing antibodies, one sample from the midbrain and one sample from the cerebellum. Tissues from these samples were processed, and transduction was measured for virus particles comprising capsid polypeptides from VAR-1, VAR-2 and VAR-3 after intravenous administration, as described in the preceding Examples. Table 7 summarized the transduction of these virus particles, relative to virus particles comprising only wild-type AAV2 capsid polypeptides delivered intravenously. The results indicate that, following intravenous administration, virus particles comprising the capsid polypeptides described herein result in transduction levels in bulk midbrain tissue of between 170- and 372-fold greater than that from virus particles comprising wild-type AAV2 capsid polypeptides, and in bulk cerebellum tissue of between 20- and 46-fold greater than that from virus particles comprising wild-type AAV2 capsid polypeptides. We note here that wild-type AAV2 had much lower transduction in the midbrain samples as compared with in the cerebellum samples. While the bulk wild-type AAV2 transduction rate was measurable in the midbrain, sufficient to establish the normalization baseline, the relative midbrain transduction rates may have additional uncertainty due to the overall low wild-type rates. This does not affect the finding that these variants transduce midbrain samples at substantially higher rates than wild-type AAV2. Finally, when compared to transduction levels in both brain samples from intravenously delivered AAV9—a capsid which has been used in approved and clinical therapies to target cells of the brain—virus particles comprising the capsid polypeptides described herein achieve relative transduction rates of 32% (VAR-1), 48% (VAR-2) and 72% (VAR-3) that of AAV9, indicating levels of transduction which are similar to those achieved by therapeutic and clinical gene therapies. Taken together with the other results described herein, these results suggest that the capsid polypeptides described herein, when incorporated into virus particles, are capable of directing transduction and increased levels of transgene expression with high efficiency both across the brain and to ocular tissues. Coupled with the low relative observed liver transduction gene therapies comprising these capsid polypeptides described herein are particularly useful for the treatment of CNS and/or ocular disorders via systemic (for example, intravenous) administration. One particular class of diseases which is amenable to treatment with gene therapies comprising the virus particles and capsid polypeptides described herein are neuronal ceroid lipofuscinoses (NCL), for example Batten disease.

1. A variant capsid polypeptide comprising a polypeptide that has at least 70, 75, 80, 85, 90, 91, 92, 93, 94, 95, 96, 97, 98, 99% or 100% identity to a VP1, VP2, or VP3 sequence of SEQ ID NO: 2, SEQ ID NO: 3, or SEQ ID NO: 4.

2. The variant capsid polypeptide of claim 1, wherein the variant is the same serotype as the polypeptide of SEQ ID NO: 2, SEQ ID NO: 3, or SEQ ID NO: 4 (AAV2).

3. The variant capsid polypeptide of claim 1, wherein the variant is a different serotype as compared to the polypeptide of SEQ ID NO: 2, SEQ ID NO: 3, or SEQ ID NO: 4 (AAV2).

4. A variant capsid polypeptide of any of the preceding claims, wherein the polypeptide comprises a variant of SEQ ID NO: 1, wherein the variant capsid polypeptide comprises a mutation that corresponds to a mutation at one or more positions of 585, 586, 587, 588, 589, 590, 591, 593, 597, 600, 608, as compared to SEQ ID NO: 1, optionally wherein the mutation comprises an insertion, a deletion, or a substitution.

5. The variant capsid polypeptide of any of the preceding claims, wherein the capsid polypeptide comprises:
a mutation that corresponds to a mutation at position 585 as compared to SEQ ID NO: 1;
a mutation that corresponds to a mutation at position 586 as compared to SEQ ID NO: 1;
a mutation that corresponds to a mutation at position 587 as compared to SEQ ID NO: 1;
a mutation that corresponds to a mutation at position 588 as compared to SEQ ID NO: 1;
a mutation that corresponds to a mutation at position 589 as compared to SEQ ID NO: 1;
a mutation that corresponds to a mutation at position 590 as compared to SEQ ID NO: 1;
a mutation that corresponds to a mutation at position 591 as compared to SEQ ID NO: 1;
a mutation that corresponds to a mutation at position 593 as compared to SEQ ID NO: 1;
a mutation that corresponds to a mutation at position 597 as compared to SEQ ID NO: 1;
a mutation that corresponds to a mutation at position 600 as compared to SEQ ID NO: 1; or
a mutation that corresponds to a mutation at position 608 as compared to SEQ ID NO: 1.

6. The variant capsid polypeptide of any of the preceding claims, wherein the capsid polypeptide comprises one or more mutations (e.g., at least 2, at least 3, at least 4, at least 5, at least 6, or all mutations) that corresponds to a mutation at position 585, 588, 589, 590, 593, 597, and 608 as compared to SEQ ID NO: 1.

7. The variant capsid polypeptide of any of the preceding claims, wherein the capsid polypeptide comprises one or more mutations (e.g., at least 2, at least 3, at least 4, at least 5, at least 6, at least 7, at least 8, or all mutations) that corresponds to a mutation at position 585, 586, 587, 588, 589, 590, 591, 593, and 600 as compared to SEQ ID NO: 1.

8. The variant capsid polypeptide of any of the preceding claims, wherein the capsid polypeptide comprises one or more mutations (e.g., at least 2, at least 3, at least 4, or all mutations) that corresponds to a mutation at position 585, 588, 590, 591, and 597 as compared to SEQ ID NO: 1.

9. The variant capsid polypeptide of any of the preceding claims, wherein the capsid polypeptide comprises one or more mutations (e.g., at least 2, at least 3, at least 4, at least 5, at least 6, or all mutations) of R585V, R588T, Q589G, A590P, A593G, T5971, and D608N, as compared to SEQ ID NO: 1.

10. The variant capsid polypeptide of any of the preceding claims, wherein the capsid polypeptide comprises one or more mutations (e.g., at least 2, at least 3, at least 4, at least 5, at least 6, at least 7, at least 8, or all mutations) of R585S, G586S, N587I, R588T, Q589A, A590P, A591G, A593G, and V600C, as compared to SEQ ID NO: 1.

11. The variant capsid polypeptide of any of the preceding claims, wherein the capsid polypeptide comprises one or more mutations (e.g., at least 2, at least 3, at least 4, or all mutations) of R585N, R588T, A590P, A591T, and T597H, as compared to SEQ ID NO: 1.

12. A variant capsid polypeptide, comprising: (a) a polypeptide of any one of SEQ ID NO: 2, SEQ ID NO: 3, or SEQ ID NO: 4; (b) the VP2 or VP3 sequence of any one of SEQ ID NO: 2, SEQ ID NO: 3, or SEQ ID NO: 4; (c) a polypeptide comprising a sequence having at least 90%, at least 95%, at least 96%, at least 97%, at least 98%, or at least 99% identity thereto (e.g., to a polypeptide of (a) or (b)), wherein said polypeptide comprises at least one (e.g., one, two, three or more, e.g., all) of the mutations associated with any of SEQ ID NO: 2 through SEQ ID NO: 4, relative to SEQ ID NO: 1; or (d) a polypeptide having at least 1, but no more than 20, no more than 19, no more than 18, no more than 17, no more than 16, no more than 15, no more than 14, no more than 13, no more than 12, no more than 10, no more than 9, no more than 8, no more than 7, no more than 6, no more than 5, no more than 3, or no more than 2 amino acid mutations relative to the polypeptide of (a) or (b), wherein said polypeptide comprises at least one (e.g., one, two, three or more, e.g., all) of the mutations associated with any of SEQ ID NO: 2 through SEQ ID NO: 4, relative to SEQ ID NO: 1.

13. A variant capsid polypeptide comprising a VP1, VP2 or VP3, or any combination thereof, that is each at least, or about, 95, 96, 97, 98 or 99% identical to a polypeptide of SEQ ID NO: 2, SEQ ID NO: 3, or SEQ ID NO: 4 and comprises all the mutation differences of any of VAR-1 through VAR-3.

14. A variant capsid polypeptide comprising a VP1, VP2 or VP3, or any combination thereof, that each has about 1 to about 20 mutations as compared to a polypeptide of SEQ ID NO: 2, SEQ ID NO: 3, or SEQ ID NO: 4 and comprises all the mutation differences of any of VAR-1 through VAR-3.

15. A variant capsid polypeptide comprising a VP1, VP2 or VP3, or any combination thereof, that each has about 1 to about 10 mutations as compared to a polypeptide of SEQ ID NO: 2, SEQ ID NO: 3, or SEQ ID NO: 4 and comprises all the mutation differences of any of VAR-1 through VAR-3.

16. A variant capsid polypeptide comprising a VP1, VP2 or VP3, or any combination thereof, that each has about 1 to about 5 mutations as compared to a polypeptide of SEQ ID NO: 2, SEQ ID NO: 3, or SEQ ID NO: 4 and comprises all the mutation differences of any of VAR-1 through VAR-3.

17. A variant capsid polypeptide comprising a VP1, VP2 or VP3 sequence of SEQ ID NO: 2, SEQ ID NO: 3, or SEQ ID NO: 4.

18. A variant capsid polypeptide consisting of the VP1, VP2 or VP3 sequence of SEQ ID NO: 2, SEQ ID NO: 3, or SEQ ID NO: 4.

19. The variant capsid polypeptide of any of the preceding claims, wherein the variant capsid polypeptide is a VP1 polypeptide, a VP2 polypeptide or a VP3 polypeptide.

20. A nucleic acid molecule encoding the variant capsid polypeptide of any one of claims 1-19.

21. The nucleic acid molecule of claim 20, wherein the nucleic acid molecule comprises a sequence of SEQ ID NO: 5, 6, 7, a fragment thereof, or a variant thereof having at least 70, 75, 80, 85, 90, 91, 92, 93, 94, 95, 96, 97, 98, 99, or 100% sequence identity thereto.

22. The nucleic acid molecule of claim 21, wherein the fragment thereof encodes a VP2 capsid polypeptide or a VP3 capsid polypeptide.

23. A virus particle (e.g., adeno-associated virus (“AAV”) particle) comprising the variant capsid polypeptide of any one of claims 1-19, or encoded by the nucleic acid molecule of any one of claims 20-22.

24. The virus particle of claim 23, comprising a nucleic acid comprising a payload (e.g., a heterologous transgene) and one or more regulatory elements.

25. A virus particle of any one of claims 23-24, wherein said virus particle exhibits increased ocular transduction, e.g., as measured in a mouse or in NHP, e.g., as described herein, relative to wild-type AAV2 (E.g., a virus particle comprising capsid polypeptides of SEQ ID NO: 1 or encoded by SEQ ID NO: 8).

26. The virus particle of claim 25, wherein the increased ocular transduction is increased retinal transduction.

27. The virus particle of any one of claims 25-26, wherein the increased ocular transduction exhibited after systemic, e.g., intravenous, administration.

28. The nucleic acid molecule of any one of claims 20-22, wherein the nucleic acid molecule is double-stranded or single-stranded, and wherein the nucleic acid molecule is linear or circular, e.g., wherein the nucleic acid molecule is a plasmid.

29. A method of producing a virus particle comprising a variant capsid polypeptide, said method comprising introducing the nucleic acid molecule of any one of claims 20-22 or 28 into a cell (e.g., a HEK293 cell), and harvesting said virus particles therefrom.

30. A method of delivering a payload (e.g., a nucleic acid) to a cell comprising contacting the cell with (a) a dependoparvovirus particle comprising the variant capsid polypeptide of any one of claims 1-19 and a payload or (b) the virus particle of any one of claims 24-27.

31. The method of claim 30, wherein the cell is an ocular cell.

32. The method of claim 31, wherein the ocular cell is in the retina.

33. A method of delivering a payload (e.g., a nucleic acid) to a subject comprising administering to the subject a dependoparvovirus particle comprising a variant capsid polypeptide of any one of claims 1-19 and the payload, or administering to the subject the virus particle of any one of claims 24-27.

34. The method of claim 33, wherein the virus particle delivers the payload to the eye.

35. The method of claim 34, wherein the virus particle delivers the payload to the retina.

36. The method of any one of claims 30-35, wherein the virus particle is administered by systemic, e.g., intravenous, administration, or by intravenous administration directly to the ophthalmic artery.

37. The variant capsid polypeptide of any one of claims 1-19, the virus particle of any one of claims 33-37, or the method of any one of claims 30-36, wherein the virus particle (e.g., the virus particle comprising the variant capsid polypeptide) delivers the payload to the eye with increased transduction in one or more regions of the eye as compared to a virus particle comprising capsid polypeptides of SEQ ID NO: 1.

38. The variant capsid polypeptide, virus particle or method of claim 37, wherein the one or more regions of the eye is the retina, and wherein the retina comprises non-macular retina.

39. The variant capsid polypeptide of any one of claims 1-19, the virus particle of any one of claims 23-27, or the method of any one of claims 30-36, wherein the virus particle (e.g., the virus particle comprising the variant capsid polypeptide) delivers the payload to the eye with increased transduction in one or more regions of the eye as compared to a virus particle comprising capsid polypeptides of SEQ ID NO: 1, and wherein the increase in transduction is at least 2-times, 4-times, 8-times, 16-times, 32-times, 64-times, 100-times, 150-times, 200-times, or 250-times greater as compared to a virus particle comprising capsid polypeptides of SEQ ID NO: 1.

40. The variant capsid polypeptide of any one of claims 1-19, the virus particle of any one of claims 23-27, or the method of any one of claims 30-36, wherein the virus particle (e.g., the virus particle comprising the variant capsid polypeptide) delivers the payload to the eye with increased transduction specificity in one or more regions of the eye as compared to a virus particle comprising capsid polypeptides of SEQ ID NO: 1, wherein the increase in transduction is at least 2-times, 4-times, 8-times, 16-times, 32-times, 64-times, 100-times, 200-times, 500-times, or 1000-times greater as compared to a virus particle comprising capsid polypeptides of SEQ ID NO: 1, and wherein the increase in transduction is specific to non-macular retina.

41. The variant capsid polypeptide, virus particle or method of any one of claims 29-40, wherein the administration to the subject is via systemic, e.g., intravenous injection, or by intravenous administration directly to the ophthalmic artery.

42. A method of treating a disease or condition in a subject, comprising administering to the subject a dependoparvovirus particle in an amount effective to treat the disease or condition, wherein the dependoparvovirus particle is a virus particle comprising the capsid polypeptide of any one of claims 1-19, or encoded by the nucleic acid of any one of claims 20-21 or 28, or is the virus particle of any one of claims 23-27.

43. A method of treating a CNS and/or ocular disease or condition in a subject, comprising administering to the subject a dependoparvovirus particle in an amount effective to treat the disease or condition, wherein the dependoparvovirus particle is a virus particle comprising the capsid polypeptide of any one of claims 1-19, or encoded by the nucleic acid of any one of claims 20-21 or 28, or is the virus particle of any one of claims 23-27, optionally wherein the disease or condition is a neuronal ceroid lipofucsinosis (NCL).

44. A cell, cell-free system, or other translation system, comprising the capsid polypeptide, nucleic acid molecule, or virus particle of any one of claims 1-27 or 37-41.

45. A method of making a dependoparvovirus (e.g., an adeno-associated dependoparvovirus (AAV) particle, comprising:
providing a cell, cell-free system, or other translation system, comprising a nucleic acid of any of claims 20-22 or 28; and
cultivating the cell, cell-free system, or other translation system, under conditions suitable for the production of the dependoparvovirus particle,
thereby making the dependoparvovirus particle.

46. The method of claim 45, wherein the cell, cell-free system, or other translation system comprises a second nucleic acid molecule and said second nucleic acid molecule is packaged in the dependoparvovirus particle, and wherein the second nucleic acid comprises a payload, e.g., a heterologous nucleic acid sequence encoding a therapeutic product.

47. The method of any one of claims 45-46, wherein the nucleic acid of any of claims 20-22 or 38 mediates the production of a dependoparvovirus particle which does not include said nucleic acid of any of claims 20-22 or 28.

48. The method of any one of claims 45-47, wherein the nucleic acid of any of claims 20-22 or 38 mediates the production of a dependoparvovirus particle at a level least 10%, at least 20%, at least 50%, or at least 100% of the production level mediated by a nucleic acid molecule encoding SEQ ID NO: 1, or at least 10% greater, at least 20% greater, at least 50% greater, or at least 100% greater than the production level mediated by a nucleic acid molecule encoding SEQ ID NO: 1.

49. A composition, e.g., a pharmaceutical composition, comprising the virus particle of any one of claims 23-27 or 37-41 or a virus particle produced by the method of any one of claims 29 or 45-48, and a pharmaceutically acceptable carrier.

50. The variant capsid polypeptide of any of claims 1-19, the nucleic acid molecule of any of claims 20-22 or 28, or the virus particle of any of claims 23-27 or 37-41 for use in treating a disease or condition in a subject.

51. The variant capsid polypeptide of any of claims 1-19, the nucleic acid molecule of any of claims 20-22 or 28, or the virus particle of any of claims 23-27 or 37-41 for use in the manufacture of a medicament for use in treating a disease or condition in a subject.
